(19) United States
(12) Patent Application Publication
(10) Pub. No.: US 2007/0111988 A1
(43) Pub. Date: May 17, 2007

(54) Selective estrogen receptor modulators

(75) Inventors: Robert Dean Dally, Indianapolis, IN (US); Jeffrey Alan Dodge, Indianapolis, IN (US); Conrad Wilson Hummel, Louisville, CO (US); Scott Alan Jones, Indianapolis, IN (US); Timothy Alan Shepherd, Indianapolis, IN (US); Owen Brendan Wallace, Zionsville, IN (US); Wayne Woodrow Weber II, Fortville, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, INDIANAPOLIS, IN (US)

(21) Appl. No.: 10/597,008

(22) PCT Filed: Jan. 18, 2005
(86) PCT No.: PCT/US05/00021

§ 371 Date: Jul. 6, 2006

Related U.S. Application Data
(60) Provisional application No. 60/538,441, filed on Jan. 22, 2004.
(60) Provisional application No. 60/582,945, filed on Jun. 25, 2004.

(51) Int. Cl. A61K 31/55; A61K 31/453; C07D 409/02; A61K 31/445

(52) U.S. Cl. 514/212.01; 514/217.03; 514/319; 514/320; 540/596; 540/609; 546/202; 546/205

(57) ABSTRACT

The present invention relates to a selective estrogen receptor modulators of formula I (I); or a pharmaceutical acid addition salt thereof; and of formula II (II); or a pharmaceutical salt thereof; useful, e.g., for treating endometriosis and/or uterine leiomyoma/leiomyomata.

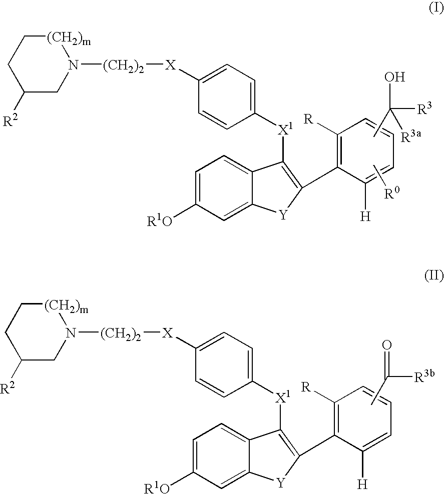

### FIELD OF THE INVENTION

The present invention is in the field of medicine, particularly in the treatment of gynecological disorders. More specifically, the present invention relates to selective estrogen receptor modulators useful to treat endometriosis and uterine fibrosis.

### BACKGROUND OF THE INVENTION

Uterine leiomyoma/leiomyomata (uterine fibroid disease) is an old and ever present clinical problem that goes under a variety of names, including uterine fibrosis, uterine hypertrophy, uterine lieomyomata, myometrial hypertrophy, fibrosis uteri, and fibrotic metritis. Essentially, uterine fibrosis is a condition where there is an inappropriate deposition of fibroid tissue on the wall of the uterus. This condition is a cause of dysmenorrhea and infertility in women.

Endometriosis is a condition of severe dysmenorrhea, which is accompanied by severe pain, bleeding into the endometrial masses or peritoneal cavity and often leads to infertility. The symptom's cause appears to be ectopic endometrial growths that respond inappropriately to normal hormonal control and are located in inappropriate tissues. Because of the inappropriate locations for endometrial growth, the tissue seems to initiate local inflammatory-like responses causing macrophage infiltration and a cascade of events leading to initiation of the painful response. Evidence suggests that a cause of uterine fibrosis and endometriosis is an inappropriate response of fibroid tissue and/or endometrial tissue to estrogen.

Many publications have appeared within the last ten years disclosing novel selective estrogen receptor modulators (SERMs), e.g., U.S. Pat. Nos. 5,484,795, 5,484,798, 5,510,358, 5,998,401 and WO 96/09040. Many of these SERMs, generally speaking, have been found to have a beneficial estrogen agonist activity in the bone and cardiovascular systems with a concomitant beneficial estrogen antagonist activity in the breast. A small, particularly useful subset of such compounds has also been found to have an estrogen antagonist effect in the uterus. A compound with this particularly useful SERM profile holds particular promise in treating uterine leiomyoma/leiomyomata and/or endometriosis.

However, the actual use of these SERM compounds, particularly in pre-menopausal women, has been hampered due to said compound's stimulatory effect on the ovaries. A great need currently exists, therefore, for new SERM compounds that behave as estrogen antagonists in the uterus that do not stimulate the ovaries.

### SUMMARY OF INVENTION

The present invention relates to a compound of formula I: 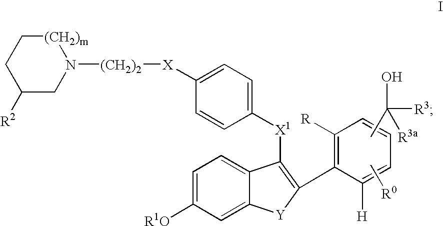
wherein:

m is 0, 1 or 2;

R⁰ is H, F or OH;

R¹ is H, SO₂(n-C₄-C₆ alkyl) or COR⁴;

R² is H or methyl provided that if m is 1 or 2, then R² must be H and that if m is 0, then R² must be methyl;

X is O or NR⁵;

Y is S or CH═CH;

R⁴ is C₁-C₆ alkyl, C₁-C₆ alkoxy, NR⁶R⁷, phenoxy, or phenyl optionally substituted with halo;

R⁵ is H or C₁-C₆ alkyl;

R⁶ and R⁷ are independently H, C₁-C₆ alkyl or phenyl;

R is H and X¹ is O, CH₂ or CO or R combines with X¹ to form a moiety of the formula: 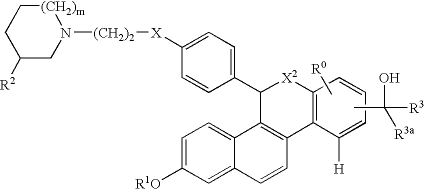
wherein m, R⁰, R¹, R², R³, R^{3a} and X are as defined above; and X² is O or S; and

R³ and R^{3a} are independently H or C₁-C₆ alkyl;

or a pharmaceutical acid addition salt thereof.

The present invention also relates to a compound of formula II: 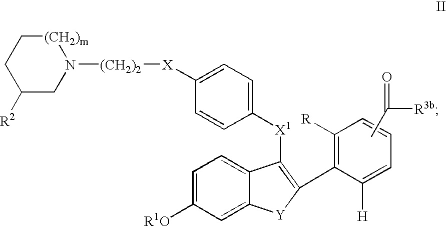
wherein:

m is 0, 1 or 2;

R¹ is H, SO₂(n-C₄-C₆ alkyl) or COR⁴;

R² is H or methyl provided that if m is 1 or 2, then R² must be H and that if m is 0, then R² must be methyl;

X is O or NR⁵;

Y is S or CH═CH;

R⁴ is C₁-C₆ alkyl, C₁-C₆ alkoxy, NR⁶R⁷, phenoxy, or phenyl optionally substituted with halo;

R⁵ is H or C₁-C₆ alkyl;

R⁶ and R⁷ are independently H, C₁-C₆ alkyl or phenyl;

R is H and X¹ is O, CH₂ or CO or R combines with X¹ to form a moiety of the formula: 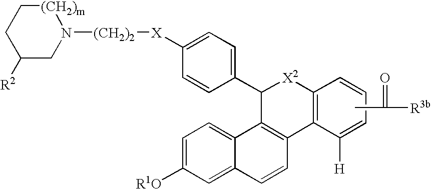
wherein X² is O or S;

R^{3b} is NR⁸R⁹ or OR¹⁰ or when R is H, R^{3b} may combine with the phenyl with which it is attached to form a moiety of the formula:

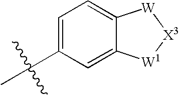

wherein
W and W¹ are CH₂ or C═O provided that at least one of W or W¹ must be C═O;
X³ is NR¹¹ or O;

R⁸ and R⁹ are independently H or C₁-C₆ alkyl or R⁸ and R⁹ may combine with the nitrogen to which they are both attached to form a morpholino, pyrollidino or piperidino ring;

R¹⁰ and R¹¹ are independently H or C₁-C₆ alkyl; or a pharmaceutical salt thereof.

The present invention also relates to a pharmaceutical composition containing a compound of formula I or II and a pharmaceutical carrier. In another embodiment, the pharmaceutical composition of the present invention may be adapted for use in treating endometriosis and/or uterine fibrosis.

The present invention also relates to methods for treating endometriosis and/or uterine fibrosis employing a compound of formula I or II.

In addition, the present invention relates to a compound of formula I of II for use in treating endometriosis and/or uterine fibrosis. The present invention is further related to the use of a compound of formula I of II for the manufacture of a medicament for treating endometriosis and/or uterine fibrosis.

The present invention further relates to a compound of formula III: 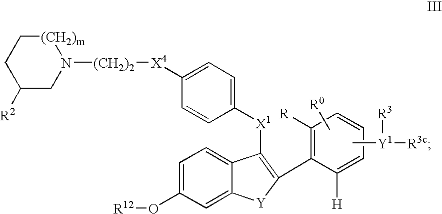
wherein:

m, R⁰, R², R³, R⁴ and Y are as defined above for a formula I compound; and

Y¹ is C═O or C(OH);

R^{3c} is absent or is H or C₁-C₆ alkyl provided that if Y¹ is C(OH), then R^{3c} is H or C₁-C₆ alkyl and that if Y¹ , is C═O, then R^{3c} is absent;

R¹² is H, C₁-C₆ alkyl, benzyl, SO₂CH₃, SO₂(n-C₄-C₆ alkyl) or COR⁴;

X⁴ is O or NR¹³;

R¹³ is H, C₁-C₆ alkyl or CO₂(C₁-C₆ alkyl);

R is H and X¹ is O, CH₂ or CO or R combines with X¹ to form a moiety of the formula: 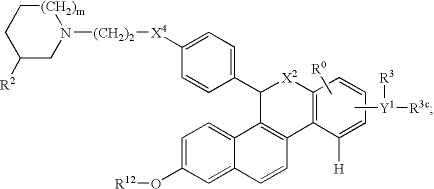
wherein m, R⁰, R², R³, R^{3c}, R¹², X², X⁴ and Y¹ are as defined above; provided that if Y¹ is C(OH), then R¹² is C₁-C₆ alkyl, SO₂CH₃ or benzyl or X⁴ is NR¹³ and R¹³ is CO₂(C₁-C₆ alkyl); or an acid addition salt thereof; useful as an intermediate to a compound of formula I.

The present invention further relates to a compound of formula IV: 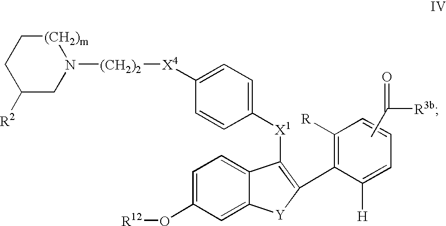
wherein m, R, R², R¹², X¹, X⁴ and Y are as defined above for a formula III compound and R^{3b} is as defined for a formula II compound; provided that if R¹² is H, SO₂(n-C₄-C₆ alkyl) or COR⁴, then X⁴ is NR¹³ and R¹³ is CO₂(C₁-C₆ alkyl); or a salt thereof; useful as an intermediate to a compound of formula II.

### DETAILED DESCRIPTION

Unless specified otherwise, reference hereafter to a “compound of formula I” includes the pharmaceutical acid addition salts thereof. Unless specified otherwise, reference hereafter to a “compound of formula II” includes the pharmaceutical salts thereof. Since the compound of formula II may contain an acidic proton, i.e., when R^{3b} is

OR¹⁰ and R¹⁰ is H, the pharmaceutical salts of the present invention include base addition and acid addition salts thereof.

The compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

For the purposes of the present invention, as disclosed and claimed herein, the following terms are defined below.

The term “halo” refers to fluoro, chloro, bromo and iodo. The term “C₁-C₆ alkyl” represents a straight, branched or cyclic hydrocarbon moiety having from one to six carbon atoms, e.g., methyl, ethyl, n-propyl, isopropyl, cyclopropyl, n-butyl, isobutyl, sec-butyl, t-butyl, cyclobutyl, pentyl, cyclopentyl, hexyl, cyclohexyl and the like. Moieties such as a cyclobutylmethylene are also included within the scope of a C₁-C₆ alkyl group. The term “C₁-C₄ alkyl” refers specifically to methyl, ethyl, n-propyl, isopropyl, cyclopropyl, n-butyl, isobutyl, sec-butyl, t-butyl and cyclobutyl. The term “n-C₄-C₆ alkyl” refers specifically to n-butyl, n-pentyl and n-hexyl. A “C₁-C₆ alkoxy” group is a C₁-C₆ alkyl moiety connected through an oxy linkage.

The term “pharmaceutical” when used herein as an adjective means substantially non-deleterious.

A pharmaceutical “acid addition salt” is a salt formed by reaction of the free base form of a compound of formula I or II with a pharmaceutical acid, such as described in the Encyclopedia of Pharmaceutical Technology, editors James Swarbrick and James C. Boylan, Vol 13, 1996 “Preservation of Pharmaceutical Products to Salt Forms of Drugs and Absorption”. Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; 1-malate; malonate; d-mandelate; 1-mandelate; methanesulfonate; 1,5 napthalenedisulfonate; 2-naphthalenesulfonate; phosphate; salicylate; succinate; sulfate; d-tartrate; 1-tartrate; and p-toluenesulfonate.

A pharmaceutical “base addition” salt is a salt formed by reaction of the free base form of a compound of formula I or II with a pharmaceutical base, such as described in the Encyclopedia of Pharmaceutical Technology, editors James Swarbrick and James C. Boylan, Vol 13, 1996 “Preservation of Pharmaceutical Products to Salt Forms of Drugs and Absorption”. Specific salt forms include, but are not limited to the: calcium, diethanolamine, diethylamine, ethylenediamine, lysine, magnesium, piperazine, potassium, sodium and tromethamine (Tris, Trizma) salts.

The term “patient” as used herein refers to female humans and non-human female animals such as companion animals (dogs, cats, horses and the like).

The terms “treating” and “treat” as used herein means alleviating, ameliorating, preventing, prohibiting, restraining, slowing, stopping, or reversing the progression or severity of a pathological condition, or sequela thereof, described herein. The term “preventing” means reducing the likelihood that the recipient of a compound of formula I will incur, further incur or develop any of the pathological conditions, or sequela thereof, described herein.

The term “a patient in need thereof” is a patient either suffering from the caimed pathological condition or sequela thereof or is a patient at a recognized risk thereof as determined by medical diagnosis, i.e., as determined by the attending physician.

As used herein, the term “effective amount” means an amount of a compound of formula I that is capable of treating the conditions described herein.

### Preferred Compounds and Embodiments of the Invention

Certain compounds of the invention are particularly interesting and are preferred. The following listing sets out several groups of preferred compounds. It will be understood that each of the listings may be combined with other listings to create additional groups of preferred compounds. The following numbering systems will be used to describe the preferred positions of the COHR³R^{3a} and COR^{3b} moieties:

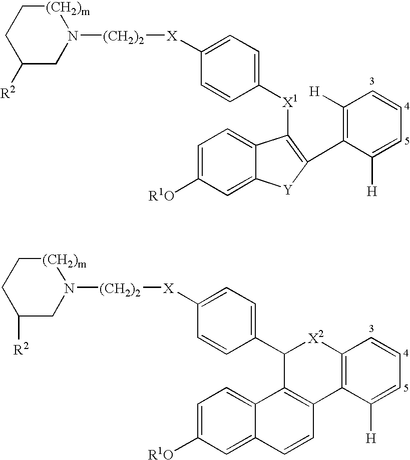

a) the compound of formula I;

b) the compound of formula II;

c) m is 1 or 2;

d) m is 1;

e) R is H;

f) R⁰ is H;

g) R¹ is H;

h) R¹ is H or COR⁴ and R⁴ is C₁-C₆ alkyl, NHCH₃ or phenyl;

i) R¹ is H or COR⁴ and R⁴ is C₁-C₄ alkyl, NHCH₃ or phenyl;

j) R³ and R^{3a} are independently H or C₁-C₄ alkyl;

k) R³ and R^{3a} are independently H or methyl;

l) the COHR³R^{3a} or COR^{3b} moiety is at position 4;

m) R^{3b} is NR⁸R⁹ and R⁸ and R⁹ are independently H or C₁-C₄ alkyl;

n) R^{3b} is OR¹⁰ and R¹⁰ is H or C₁-C₄ alkyl;

o) the COR^{3b} moiety is at position 3 or 4;

p) R is H and R^{3b} combines with the phenyl with which it is attached to form: 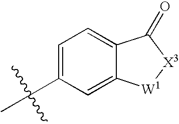
and W¹ is CH₂ and X³ is NR¹¹ and R¹¹ is H;

q) R is H and R^{3b} combines with the phenyl with which it is attached to form: 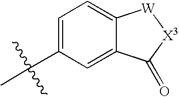
and R¹¹ is H or C₁-C₄ alkyl;

r) R is H and R^{3b} combines with the phenyl with which it is attached to form: 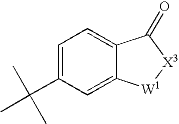
and R¹¹ is H or C₁-C₄ alkyl;

s) R combines with X¹ to form a moiety of the formula:

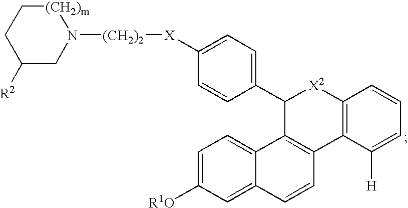

t) R combines with X¹ to form a moiety of the formula: 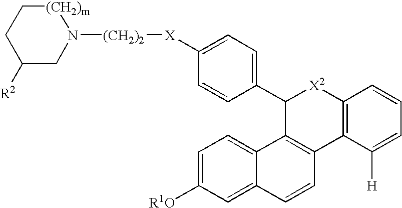
and X² is 0;

u) R⁸ and R⁹ are independently H or C₁-C₆ alkyl;

v) X is O;

w) X is NR⁵ and R⁵ is H or methyl;

x) X¹ is O or CH₂;

y) X¹ is O;

z) Y is CH═CH;

aa) the hydrochloride salt form.

With respect to the chiral center designated below: 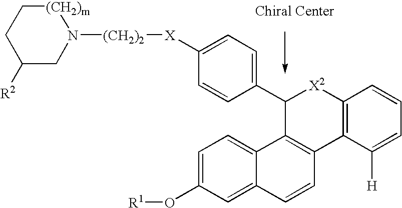
an enantiomeric excess (ee) of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column (see, e.g., J. Jacques, et al., “Enantiomers, Racemates, and Resolutions”, John Wiley and Sons, Inc., 1981; E. L. Eliel and S. H. Wilen, “Stereochemistry of Organic Compounds”, (Wiley-Interscience 1994), and European Patent Application No. EP-A-838448, published Apr. 29, 1998). Of course, the preferred enantiomer is that which possesses favorable activity in the biological assays disclosed herein. In order to verify the identify of the preferred enantiomer in any given racemic mixture, the activity of the individual isomers should be verified in the biological assays described herein.

The preferred patient of treatment is a female human.

The compound of formula I is preferably formulated in a dosage unit form, i.e., in an individual delivery vehicle, for example, a tablet or capsule, prior to administration to the recipient woman.

The compound of formula I is preferably administered orally.

Synthesis

The compound of formula I may be prepared as described in the following Schemes and Examples.

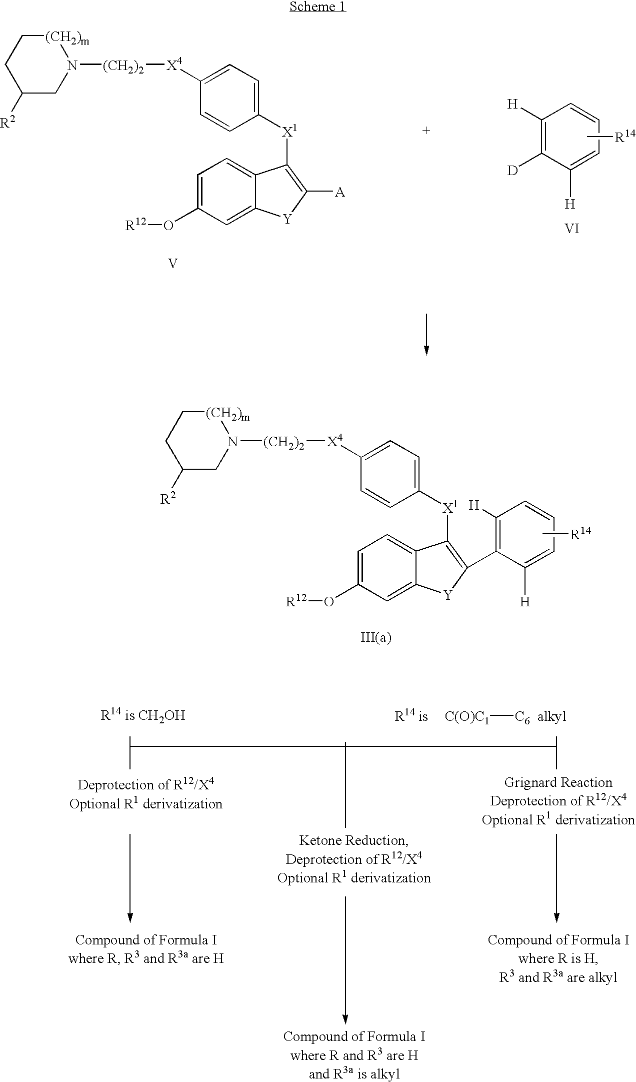

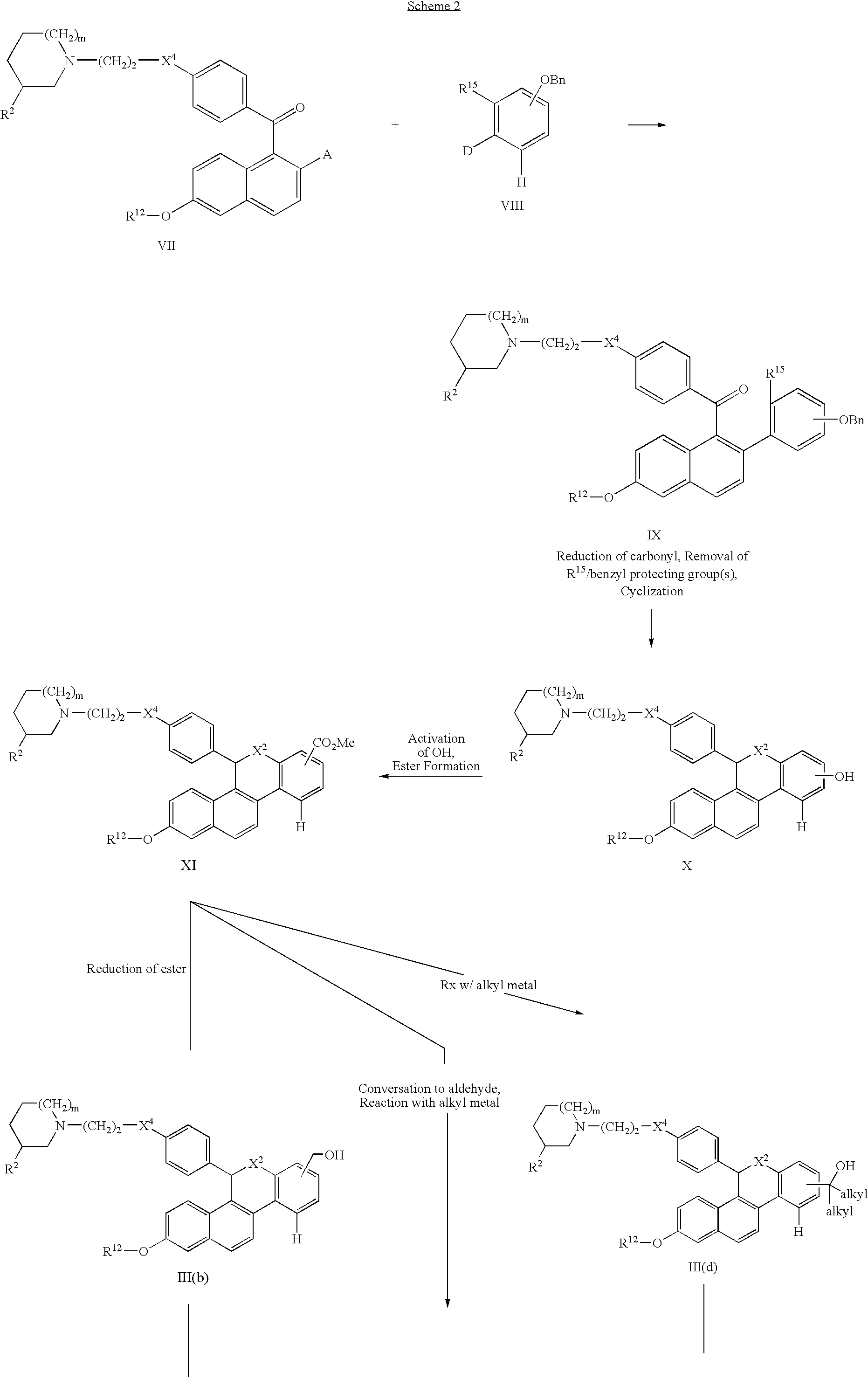

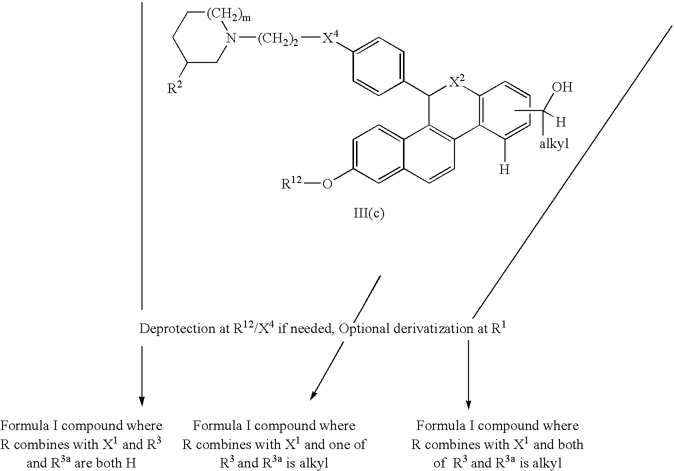

In Scheme 1, where R⁰ is H and R¹⁴ is CH₂OH or C(O)C₁-C₆ alkyl, the synthesis of a compound of formula I where R is H is illustrated. A compound of formula VI is reacted with a compound of formula V under usual “Suzuki” or “Stille” reaction conditions, i.e., wherein one of substituent “A” or “D” is a boronic acid/ester or alkyl stannane moiety and the other is a leaving group, e.g., chloro, bromo or iodo or a sulfonate group such as trifluoromethyl sulfonate to form a compound of formula II(a). A compound of formula I where R³ and R^{3a} are both hydrogen, where one of R³ and R^{3a} is alkyl and where both of R³ and R^{3a} are alkyl may be accessed as illustrated in the Scheme and as taught below in the working examples.

In Scheme 2, where R⁰ is H and R¹⁵ is alkyl or benzyl protected thio or hydroxy and “Bn” denotes benzyl, a compound of formula VII is reacted with a compound of formula VIII under usual “Suzuki” or “Stille” reaction conditions as described above to form a compound of formula IX. The ketone in the formula IX compound may then be reduced to the corresponding alcohol employing typical procedures for such a transformation (see working examples below). The benzyl protecting group along with the hydroxy or thio protecting group at R¹⁵ may then be removed under conditions that also promote cyclization (see working examples below) to provide the compound of formula X. The free hydroxy group found in the compound of formula X may then be activated towards nucleohilic displacement, e.g., by formation of the triflate. Said activated hydroxy compound may then be reacted with carbon monoxide under transition metal catalysis (e.g., Pd(Oac)₂) in the presence of methanol to afford the corresponding methyl ester of formula XI. Said ester may then be reduced under standard conditions (e.g., with LiAlH₄) to form the compound of formula III(b). A compound of formula I or III where only one of R³ or R^{3a} is alkyl may be prepared by reacting the aforementioned ester with DIBAL to yield the corresponding aldehyde, followed by reaction with at least one equivalent of an alkyl metal (e.g., alkyl lithium). A compound of formula I or III where R³ and R^{3a} are alkyl may be prepared by reacting the aforementioned ester with at least two equivalents of an alkyl metal (e.g., alkyl lithium).

The compound of formula II may be prepared as described in the following Schemes and Examples.

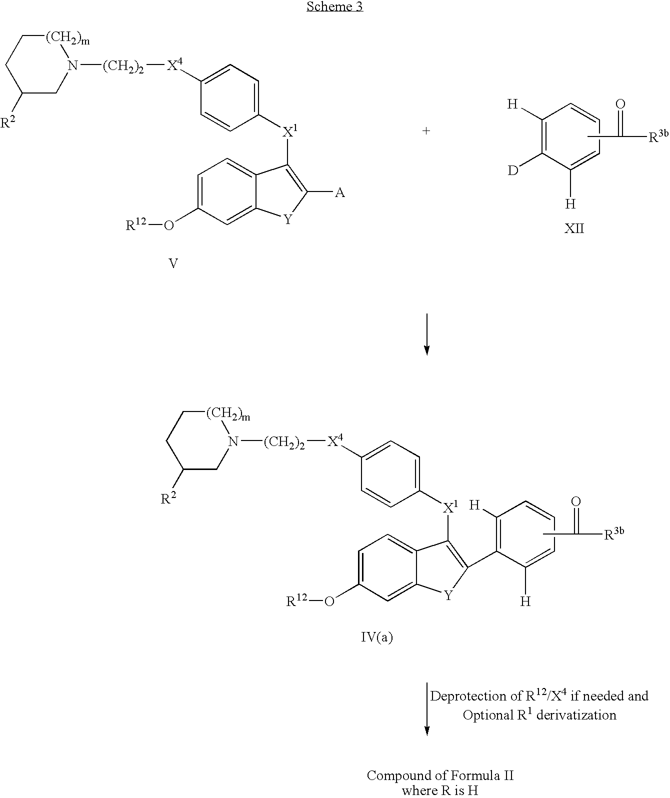

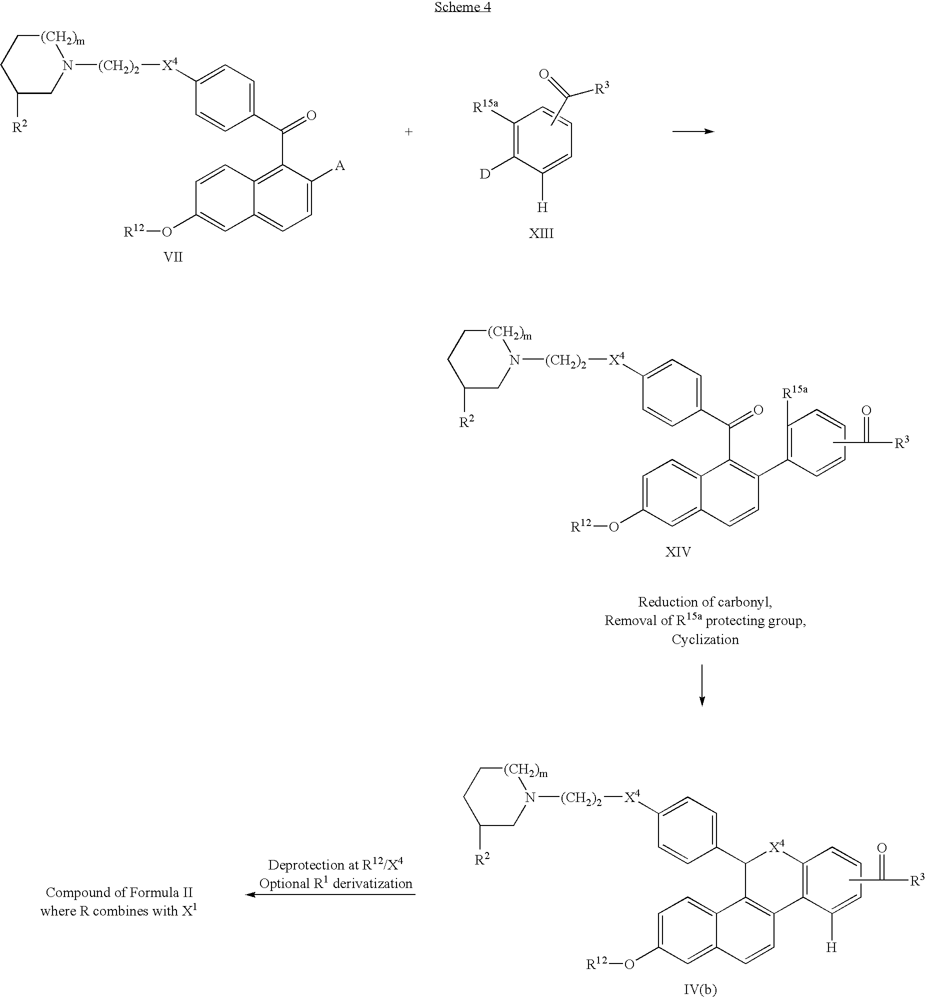

In Scheme 3, a compound of formula XII is reacted with a compound of formula V as described above in Scheme 1 for the reaction of a compound of formula V with a compound of formula VI to to give the corresponding compound of formula II or IV(a).

In Scheme 4, where R^{15a} is fluoro or R¹⁵, a compound of formula VII is reacted with a compound of formula XIII as described in Scheme 2 for the reaction of a compound of formula VII with a compound of formula VIII. When R^{15a} is protected hydroxy, said hydroxy group is typically removed in order to promote the following reduction/cyclization reaction. Said protecting group may be removed via standard procedure, e.g., those described in the latest edition of Greene, Protective Groups in Organic Synthesis, John Wiley & Sons, New York, N.Y. (Greene). After removal of the hydroxy protecting group (when present), the keto group found in the resulting product compound of formula XIV may then be reduced under standard conditions, e.g., employing borane to provide the corresponding alcohol. This reduced product may then be cyclized under standard conditions, e.g., when R^{15a} is F, base catalyzation with potassium t-butoxide or when R^{15a} is other than F, acid catalyzation with HCl, to provide the corresponding compound of formula II or IV(b).

When R¹² in the formula III and IV compounds is SO₂CH₃, C₁-C₆ alkyl or benzyl (preferably methyl, benzyl or SO₂CH₃) said hydroxy protecting groups may be removed under standard conditions (see, e.g., the procedures that follow or Greene) to provide the corresponding compound of formula I, II, III or IV where R¹ is H. Similarly, when X⁴ is NR¹³ and R¹³ is CO₂(C₁-C₆ alkyl), said amino protecting group may also be removed as taught in Greene. A formula I, II, III or IV compound where R¹ is H may be further derivatized employing standard acylation or sulfonylation methodology to prepare a compound of formula I, II, III or IV where R¹ is COR⁴ or SO₂(n-C₄-C₆ alkyl).

### Preparation 1

### Trifluoromethanesulfonic acid 6-methoxy-1-[4-(2-piperidin-1-yl-ethoxy)-phenoxy]-naphthalen-2-yl ester

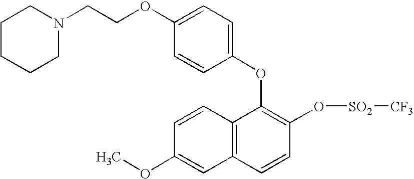

Add 6-methoxynaphthalene-2-ol (20 g, 114.8 mmol) to dimethylformamide (DMF, 250 mL) at ambient temperature followed by N-bromosuccinimide (NBS, 21.5 g, 120 mmol) over a 30 minute period. After 45 minutes, dilute with water (800 mL), collect and dry the precipitate to provide 25.5 g (87%) of 1-bromo-6-methoxy-naphthalen-2-ol.

Add 1-bromo-6-methoxy-naphthalen-2-ol (66.7 g, 264 mmol), potassium carbonate (K₂CO₃, 40.0 g, 290 mmol) and benzyl bromide (49.6 g, 290 mmol) to DMF (800 mL). Stir the mixture at ambient temperature for 1 hour. Add water (400 mL) to precipitate the product. Collect the precipitate and wash the filter cake with heptane (3×125 mL) then dry to provide 83.7 g of 2-benzyloxy-1-bromo-6-methoxy-naphthalene (86.2%).

Combine toluene (200 mL), 2-benzyloxy-1-bromo-6-methoxy-naphthalene (30 g, 87.4 mmol), 4-(2-piperidin-1-yl-ethoxy)phenol (23.2 g, 105 mmol) and cesium carbonate (34.4 g, 105 mmol), and heat the mixture to reflux. Remove a portion of the toluene (100 mL). Add ethyl acetate (390 mg, 4.37 mmol) and copper triflate benzene complex (2.20 g, 4.37 mmol) to the reaction mixture and stir for 5 minutes. Remove the solvent by distillation and heat the resulting residue to 174° C. for 1.5 hours. Dissolve the residue in a mixture of ethyl acetate (200 mL) and aqueous HCl (1 N, 90 mL). Separate and concentrate the organics to a residue. Column chromatograph the residue to give 12.4 g of 1-{2-[4-(2-benzyloxy-6-methoxy-naphthalen-1-yloxy)-phenoxy]-ethyl}-piperidine (30%).

Add 1-{2-[4-(2-benzyloxy-6-methoxy-naphthalen-1-yloxy)-phenoxy]-ethyl}-piperidine (12.4 g, 25.5 mmol) to a methanol/ethyl acetate mixture (1:1, 490 mL) and heat to form a solution. Remove the heat and add ammonium formate (4.83 g, 76.6 mmol) and Pd(OH)₂ on carbon (20% ww, 1.58 g, 1.12 mmol). Reflux for 50 minutes then filter the mixture. Concentrate the filtrate to provide 9.9 g of 6-methoxy-1-[4-(2-piperidin-1-yl-ethoxy)-phenoxy]-naphthalene-2-ol (98.5%).

Cool dichloromethane (290 mL), triethylamine (3.08 g, 30.4 mmol) and 6-methoxy-1-[4-(2-piperidin-1-yl-ethoxy)-phenoxy]-naphthalene-2-ol (9.2 g, 23.4 g) to −50° C. and add trifluoromethane sulfonic acid anhydride (7.26 g, 25.7 mmol). Stir the resulting mixture at −50° C. for 2 hours then allow the mixture to warm to ambient temperature before stirring for an additional hour. Add brine (150 mL) and separate the organics. Wash the organics with NaHCO₃ then dry before concentrating to a residue. Crystallize the residue with ethyl ether—hexanes to provide 11.2 g of the title compound (90.9%).

Preparation 2

### Trifluoromethanesulfonic acid 6-benzyloxy-1-[4-(2-piperidin-1-yl-ethoxy)-phenoxy]-naphthalen-2-yl ester

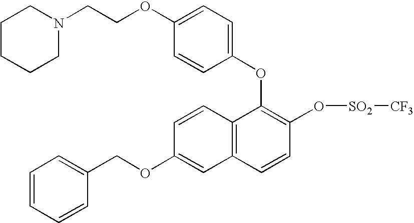

Add 2M hydrogen chloride in ether (1.5 mL, 3 mmol) to a solution of trifluoromethanesulfonic acid 6-methoxy-1-[4-(2-piperidin-1-yl-ethoxy)-phenoxy]-naphthalen-2-yl ester (1.07 g, 2.04 mmol) in dichloromethane (20 mL) and remove solvent under vacuum. Dissolve the hydrochloride salt in dichloromethane (40 mL) and cool in ice bath. Add boron tribromide (0.58 mL, 6.12 mmol), stir 3.5 hours, warm to ambient temperature and stir for 15 minutes, cool in ice bath and quench with ice cold saturated aqueous sodium bicarbonate. Extract aqueous layer with dichloromethane, combine organic layers and dry with magnesium sulfate, remove solvent under vacuum and chromatograph on silica gel using dichloromethane/methanol mixtures to give 99 mg (95%) of trifluoromethanesulfonic acid 6-hydroxy-1-[4-(2-piperidin-1-yl-ethoxy)-phenoxy]-naphthalen-2-yl ester. Mass spectrum (ion spray): m/z=512 (M+1).

Combine trifluoromethanesulfonic acid 6-hydroxy-1-[4-(2-piperidin-1-yl-ethoxy)-phenoxy]-naphthalen-2-yl ester (247 mg, 0.48 mmol), triphenylphosphine (190 mg, 0.725 mmol), benzyl alcohol (0.075 mL, 0.725 mmol) and tetrahydrofuran (5 mL) in an ice bath. Add diisopropyl azodicarboxylate (0.14 mL, 0.725 mmol), stir 1 hour, warm to ambient temperature and stir 30 minutes. Dilute with ethyl acetate and wash with 50% saturated aqueous sodium bicarbonate and saturated aqueous sodium chloride, dry with magnesium sulfate and remove solvent under vacuum. Chromatograph on silica gel with dichloromethane/methanol mixtures to give 213 mg (73%) of the title compound: Mass spectrum (ion spray): m/z=602 (M+1).

### Preparation 3

### 3-{6-Hydroxy-1-[4-(2-piperidin-1-yl-ethoxy)-phenoxy]-naphthalen-2-yl}-benzonitrile hydrochloride

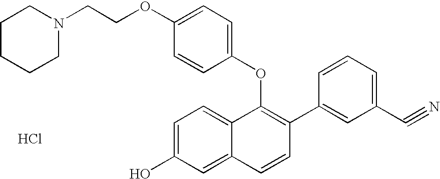

Combine trifluoromethanesulfonic acid 6-methoxy-1-[4-(2-piperidin-1-yl-ethoxy)-phenoxy]-naphthalen-2-yl ester (740 mg, 1.41 mmol), 3-cyanobenzeneboronic acid (620 mg, 4.23 mmol), palladium(II)acetate (31.6 mg, 0.14 mmol), tricyclohexylphosphine (59.3 mg, 0.21 mmol), cesium fluoride (1.93 g, 12.68 mmol) and acetonitrile (15 mL) and heat at 90° C. After 10 minutes, cool to ambient temperature, filter and remove solvent under vacuum. Dissolve in dichloromethane and filter through Celite. Chromatograph on silica gel with dichloromethane/methanol mixtures and add 1M hydrogen chloride in ether (1.5 mL) to give 730 mg (100%) of 3-{6-methoxy-1-[4-(2-piperidin-1-yl-ethoxy)-phenoxy]-naphthalen-2-yl}-benzonitrile hydrochloride.

Dissolve 3-{6-methoxy-1-[4-(2-piperidin-1-yl-ethoxy)-phenoxy]-naphthalen-2-yl}-benzonitrile hydrochloride (730 mg, 1.411 mmol) in dichloromethane (20 mL), cool in an ice bath and add 1M boron tribromide in dichloromethane (4.23 mL, 4.23 mmol). Let slowly warm to ambient temperature over 18 hours, quench with saturated sodium bicarbonate, dry organic layer with magnesium sulfate, filter and chromatograph on silica gel with dichloromethane/methanol mixtures. Combine fractions containing product, add 1M hydrogen chloride in ether (1.5 mL) and remove solvent under vacuum to give 670 mg (98%) of the title compound. Mass spectrum (ion spray): m/z=465.2 (M+1).

### EXAMPLE 1

### 3-{6-Hydroxy-1-[4-(2-piperidin-1-yl-ethoxy)-phenoxy]-naphthalen-2-yl}-benzamide hydrochloride

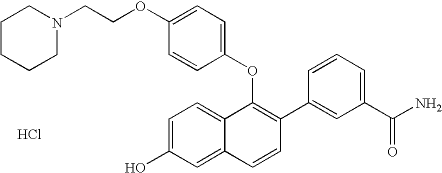

Heat a solution of 3-{6-hydroxy-1-[4-(2-piperidin-1-yl-ethoxy)-phenoxy]-naphthalen-2-yl}-benzonitrile hydrochloride (68 mg, 0.14 mmol) in concentrated hydrochloric acid (8 mL) at 70° C. for 2 hours, cool to ambient temperature and remove the solvent under reduced pressure. Dissolve in 5% methanol/dichloromethane and wash with saturated sodium bicarbonate, saturated sodium chloride, dry with magnesium sulfate, filter and chromatograph on silica gel with dichloromethane/methanol mixtures. Combine fractions containing product and add 1M hydrogen chloride in ether (0.5 mL). Remove the solvent under reduced pressure to give 62 mg (88%) of the title compound. Mass spectrum (ion spray): m/z=483.3 (M+1).

### EXAMPLE 2

### 3-{6-Hydroxy-1-[4-(2-piperidin-1-yl-ethoxy)-phenoxy]-naphthalen-2-yl}-benzoic acid methyl ester hydrochloride

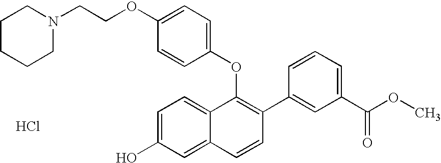

Heat a suspension of 3-{6-methoxy-1-[4-(2-piperidin-1-yl-ethoxy)-phenoxy]-naphthalen-2-yl}-benzonitrile hydrochloride (190 mg, 0.37 mmol) in concentrated hydrochloric acid (5 mL) in a sealed vessel at 130° C. for 3.5 hours, then cool to ambient temperature and remove the solvent under reduced pressure. Coevaporate with methanol (3×). Redissolve in methanol and add 4M hydrogen chloride in dioxane (1 mL). Reflux for 1 hour, then cool to ambient temperature and evaporate under reduced pressure. Dissolve in 5% methanol/dichloromethane and wash with saturated sodium bicarbonate, dry with magnesium sulfate and chromatograph on silica gel with dichloromethane/methanol mixtures. Combine fractions containing product and add 1M hydrogen chloride in ether (0.25 mL) and remove the solvent under reduced pressure to give 140 mg (73%) of the title compound.

### EXAMPLE 3

### 3-{6-Hydroxy-1-[4-(2-piperidin-1-yl-ethoxy)-phenoxy]-naphthalen-2-yl}-benzoic acid trifluoroacetate

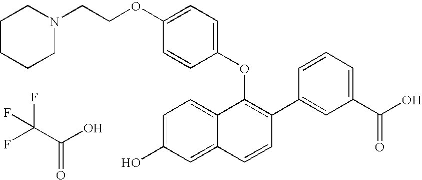

Heat a suspension of 3-{6-methoxy-1-[4-(2-piperidin-1-yl-ethoxy)-phenoxy]-naphthalen-2-yl}-benzonitrile hydrochloride (100 mg, 0.20 mmol) in concentrated hydrochloric acid (5 mL) in a sealed vessel at 130° C. for 3.5 hours, then cool to ambient temperature and remove the solvent under reduced pressure. Chromatograph on reversed phase C-18 silica gel with water/acetonitrile/trifluoroacetic acid mixtures. Combine fractions containing product and remove the solvent under reduced pressure to give 44 mg (37%) of the title compound. Mass spectrum (ion spray): m/z=484.1 (M+1).

### EXAMPLE 4

### 4-{6-Methoxy-1-[4-(2-piperidin-1-yl-ethoxy)-phenoxy]-naphthalen-2-yl}-benzoic acid methyl ester hydrochloride

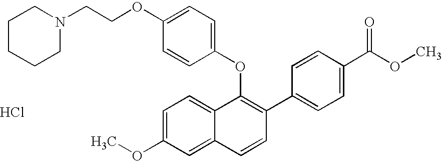

Using a method similar to that described for the preparation of 3-{6-methoxy-1-[4-(2-piperidin-1-yl-ethoxy)-phenoxy]-naphthalen-2-yl}-benzonitrile hydrochloride, obtain 254 mg (79%) of the title compound using [1,1′-bis(diphenylphosphino)-ferrocene]dichloropalladium(II)complex with dichloromethane (1:1) (480 mg, 1.0 equivalent) as catalyst system and 4-methoxycarbonylphenyl boronic acid. Mass spectrum (ion spray): m/z=512 (M+1).

### EXAMPLE 5

### 4-{6-Hydroxy-1-[4-(2-piperidin-1-yl-ethoxy)-phenoxy]-naphthalen-2-yl}-benzoic acid methyl ester hydrochloride

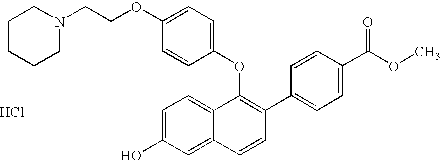

Using a method similar to that described for the preparation of 3-{6-hydroxy-1-[4-(2-piperidin-1-yl-ethoxy)-phenoxy]-naphthalen-2-yl}-benzonitrile hydrochloride, convert 4-{6-methoxy-1-[4-(2-piperidin-1-yl-ethoxy)-phenoxy]-naphthalen-2-yl}-benzoic acid methyl ester hydrochloride to 49 mg (25%) of the title compound. Mass spectrum (ion spray): m/z=498 (M+1).

### EXAMPLE 6

### 4-{6-Hydroxy-1-[4-(2-piperidin-1-yl-ethoxy)-phenoxy]-naphthalen-2-yl}-benzoic acid hydrochloride

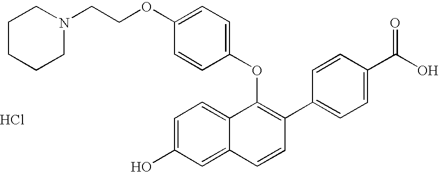

Add 1N aqueous sodium hydroxide solution (0.15 mL) to a solution of 4-{6-hydroxy-1-[4-(2-piperidin-1-yl-ethoxy)-phenoxy]-naphthalen-2-yl}-benzoic acid methyl ester hydrochloride (35 mg, 0.071 mmol) in tetrahydrofuran (1 mL), stir and heat at 60° C. After 3 hours, cool to ambient temperature and remove solvent under a stream of nitrogen. Chromatograph on reversed phase silica gel with dilute aqueous hydrochloric acid/acetonitrile mixtures to give 4.8 mg (13%) of the title compound. Mass spectrum (ion spray): m/z=484 (M+1).

### EXAMPLE 7

### 3-{6-Methoxy-1-[4-(2-piperidin-1-yl-ethoxy)-phenoxy]-naphthalen-2-yl}-N,N-dimethyl-benzamide

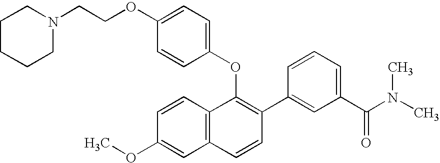

Combine N,N-dimethylbenzamide-3-boronic acid (300 mg, 1.55 mmoL), trifluoro-methanesulfonic acid 6-methoxy-1-[4-(2-piperidin-1-yl-ethoxy)-phenoxy]-naphthalen-2-yl ester (WO 2004/009086; 273 mg, 0.52 mmoL), cesium fluoride (710 mg, 4.68 mmoL) and acetonitrile (5 mL) in a 50 mL flame-dried flask fitted with a reflux condenser. In a separate flask combine palladium(II) acetate (11 mg, 0.05 mmoL) and tricyclohexylphosphine (21 mg, 0.075 mmoL). Add acetonitrile (2.5 mL) and sonicate for 10 minutes under nitrogen. Add the catalyst slurry to the mixture of substrates and heat in a 90° C. oil bath for 40 minutes. Cool the suspension to room temperature and filter through GF/F filter paper. Rinse the filter cake with acetonitrile and concentrate the filtrate in vacuo. Partition the residue between ethyl acetate (25 mL) and 5% aqueous sodium carbonate (25 mL). Separate and wash the organic layer with saturated aqueous NH₄Cl, and saturated aqueous NaCl. Dry the organic layer (Na₂SO₄), filter, and evaporate to obtain 440 mg of crude material. Chromatograph the residue on a SiO₂ column eluting the material with 2.5% methanol in dichloromethane to give 258 mg (94%) of the title compound: mass spectrum (ion spray): m/z=525.4 (M+H).

### EXAMPLE 8

### 3-{6-Hydroxy-1-[4-(2-piperidin-1-yl-ethoxy)-phenoxy]-naphthalen-2-yl}-N,N-dimethylbenzamide, hydrochloride salt

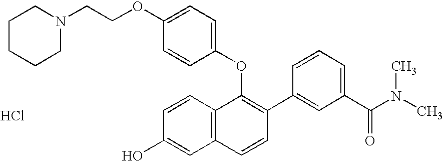

Dissolve 3-{6-methoxy-1-[4-(2-piperidin-1-yl-ethoxy)-phenoxy]-naphthalen-2-yl}-N,N-dimethyl-benzamide in ethyl acetate (10 mL) and diethyl ether (5 mL). Add 2M HCl in diethyl ether (250 μL, 500 μmol). Concentrate the slurry and dry in vacuo. Dilute the residue in dichloromethane (10 mL) and blanket with nitrogen. Cool the solution to 3° C. with external ice bath and treat with BBr₃ (250 μL, 2.65 mmoL). After 2 hours, dilute the reaction mixture with ethyl acetate (40 mL), methanol (5 mL), and saturated aqueous NaHCO₃ (20 mL). Separate the layers and back extract the aqueous layer with ethyl acetate (10 mL). Combine the organic layers and wash with a 1:1 solution of water and brine (10 mL). Dry with Na₂SO₄, filter, and concentrate in vacuo. Chromatograph the residue (264 mg) on a SiO₂ column eluting the material with methanol in dichloromethane (2.5 to 10%). Dissolve the free base in diethyl ether (5.0 mL), ethyl acetate (6.0 mL) and methanol (1.0 mL) and add 2M HCl in diethyl ether. Collect the precipitate on filter paper, rinse with diethyl ether and dry in vacuo (<2 mm of Hg) at 65° C. for 48 hours to give 175 mg (65%) of the title compound: mass spectrum (ion spray): m/z=511.3 (M+1).

### Preparation 4

### 5-(4,4,5,5-Tetramethyl-[1,3,2]dioxaborolan-2-yl)-2,3-dihydro-isoindol-1-one

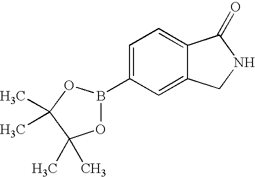

Combine 4-bromo-2-bromomethyl-benzoic acid methyl ester (3.0 g, 9.7 mmol) and 7M NH₃ in methanol (100 mL, 700 mmol) in a sealed tube and heat in a 40° C. oil bath for 18 hours. Cool the resulting suspension to room temperature and filter to obtain 1.5 g of 5-bromo-2,3-dihydro-isoindol-1-one (72%).

Combine 5-bromo-2,3-dihydro-isoindol-1-one (1.1 g, 5.0 mmol), bis-pinocalatodiboron (1.4 g, 5.5 mmol), [1,1′-Bis(diphenylphosphino)-ferocene]dichloropaladium(II) complex with dichloromethane (408 mg, 0.5 mmol) and potassium acetate (1.5 g, 15.0 mmol) in a 200 mL flask with a septum. Add dimethyl sulfoxide (27 mL) and heat in a 90° C. oil bath for 18 hours. Cool the resulting slurry to room temperature and dilute with water (100 mL). Extract the resulting slurry with dichloromethane (3×75 mL). Wash the combined organic layers with brine (40 mL), dry (Na₂SO₄), filter and concentrate in vacuo to obtain 1.6 g of a mixture of the title product and bis-pinocalatodiboron (1:0.05), which is used without further purification.

### EXAMPLE 9

### 5-{6-Methoxy-1-[4-(2-piperidin-1-yl-ethoxy)-phenoxy]-naphthalen-2-yl}-2,3-dihydro-isoindol-1-one

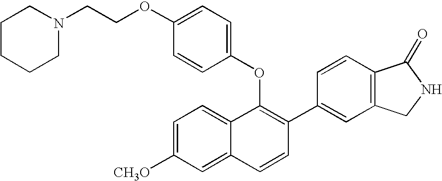

Combine trifluoro-methanesulfonic acid 6-methoxy-1-[4-(2-piperidin-1-yl-ethoxy)-phenoxy]-naphthalen-2-yl ester (800 mg, 1.5 mmol), 5-(4,4,5,5-Tetramethyl-[1,3,2]dioxaborolan-2-yl)-2,3-dihydro-isoindol-1-one (1.2 g, 3.0 mmol) and acetonitrile (25 mL) in a 100 mL flask with septum. In a separate flask combine palladium(II) acetate (67 mg, 0.3 mmol) and tricyclohexylphisphine (129 mg, 0.5 mmol). Add acetonitrile (15 mL) and sonicate for 10 minutes under nitrogen. Add the catalyst slurry and cesium fluoride (2.1 mg, 13.7 mmol) to mixture of substances and heat in a 78° C. oil bath for 18 hours. Cool the resulting suspension to room temperature and filter through packed celite. Rinse the celite with ethyl acetate. Concentrate the filtrate and pre-adsorb the crude product onto silica gel. Chromatograph the residue on a SiO₂ column eluting the material with methanol in dichloromethane (0 to 30%) to give 313 mg of 5-{6-methoxy-1-[4-(2-piperidin-1-yl-ethoxy)-phenoxy]-naphthalen-2-yl}-2,3-dihydro-isoindol-1-one (40%).

### EXAMPLE 10

### 5-{6-Methoxy-1-[4-(2-piperidin-1-yl-ethoxy)-phenoxy]-naphthalen-2-yl}-2,3-dihydro-isoindol-1-one Hydrochloride

Dissolve 5-{6-methoxy-1-[4-(2-piperidin-1-yl-ethoxy)-phenoxy]-naphthalen-2-yl}-2,3-dihydro-isoindol-1-one (313 mg, 0.6 mmol) in dichloromethane (2 mL). Treat the resulting solution with 1M HCl in diethyl ether (10 mL, 10 mmol). Concentrate the resulting suspension in vacuo to obtain 333 mg of the title compound (99%).

### EXAMPLE 11

### 5-{6-Hydroxy-1-[4-(2-piperidin-1-yl-ethoxy)-phenoxy]-naphthalen-2-yl}-2,3-dihydro-isoindol-1-one Hydrochloride

Dissolve 5-{6-methoxy-1-[4-(2-piperidin-1-yl-ethoxy)-phenoxy]-naphthalen-2-yl}-2,3-dihydro-isoindol-1-one hydrochloride (333 mg, 0.6 mmol) in dichloromethane (12 mL) and cool to 0° C. in an ice-bath. Treat solution with 1M boron tribromide in dichloromethane (2.4 mL, 2.4 mmol), drop wise over 5 minutes and stir for 1.5 hours at 0° C. Add saturated aqueous sodium bicarbonate solution (10 mL) at 0° C. and warm to room temperature. Separate the resulting layers and extract the aqueous layer with ethyl acetate (5×15 mL). Wash the combined organic layers with brine, dry (Na₂SO₄) and filter. Concentrate the filtrate and pre-adsorb the crude product onto silica gel. Chromatograph the residue on a SiO₂ column eluting with methanol in dichloromethane (0 to 40%) to give 122 mg of 5-{6-Methoxy-1-[4-(2-piperidin-1-yl-ethoxy)-phenoxy]-naphthalen-2-yl}-2,3-dihydro-isoindol-1-one. Dissolve the free-base in dichloromethane (10 mL) and treat with 1M HCl in diethyl ether (10 mL, 10 mmol). Concentrate in vacuo to obtain 130 mg of the title compound (41%): mass spectrum (ion spray): m/z=495.2 (M+H—HCl).

### Preparation 5

### 6-Bromo-2,3-dihydro-isoindol-1-one

Place 5-bromo-2-methyl-benzoic acid (1.0 g, 4.7 mmol) in a 200 mL flask under an N₂ atmosphere and add methanol via syringe. Add a 2M solution of diazomethyl-trimethyl-silane in hexane (3.5 mL, 23.0 mmol) drop wise over 10 minutes and stir for 1 hour at room temperature. Add glacial acetic acid (16 mL) and stir for 45 minutes. Dilute with ethyl acetate (100 mL) and wash with 1M aqueous sodium hydroxide solution (30 mL), saturated aqueous sodium bicarbonate solution (30 mL) and brine (30 mL). Dry the organic layer (Na₂SO₄), filter and concentrate in vacuo to obtain 1.01 g of 5-bromo-2-methyl-benzoic acid methyl ester (99%).

Place 5-bromo-2-methyl-benzoic acid methyl ester (1.04 g, 4.5 mmol) in a 50 mL flask under a N₂ atmosphere and add carbon tetrachloride (15 mL). Add N-bromo-succinamide (1.49 g, 8.3 mmol) and 2,2′-azobisisobutyronitrile (40 mg, 0.2 mmol) and fit flask with a condenser and reflux for 4 hours. Cool to room temperature and filter. Concentrate the filtrate and pre-adsorb the crude product onto silica gel. Chromatograph the residue on a SiO₂ column eluting with dichloromethane in hexane (0 to 50%) to obtain 977 mg of 5-bromo-2-bromomethyl-benzoic acid methyl ester (70%).

Using 5-bromo-2-bromomethyl-benzoic acid methyl ester (0.984 g, 3.20 mmol) and the procedure described in the 1^{st} paragraph for the alternative procedure for 5-(4,4,5,5-tetramethyl-[1,3,2]dioxaborolan-2-yl)-2,3-dihydro-isoindol-1-one, prepare 509 mg of the title compound(75%).

### EXAMPLE 12

### 6-{6-Methoxy-1-[4-(2-piperidin-1-yl-ethoxy)-phenoxy]-naphthalen-2-yl}-2,3-dihydro-isoindol-1-one

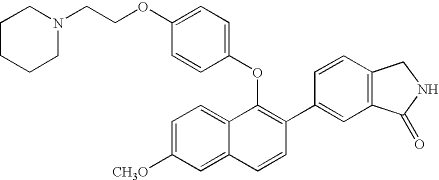

Combine 6-bromo-2,3-dihydro-isoindol-1-one (0.200 g, 0.94 mmol), bis-pinocalatodiboron (0.264 g, 1.04 mmol), palladium(II) acetate (16 mg, 0.07 mmol) and tricyclohexylphosphine (26 mg, 0.09 mmol) in a 50 mL flask. Add acetonitrile (10 mL) and cesium fluoride (0.428 g, 2.82 mmol); fit flask with condenser and heat in a 90° C. oil bath for 1 hour. Cool to room temperature and add trifluoro-methanesulfonic acid 6-methoxy-1-[4-(2-piperidin-1-yl-ethoxy)-phenoxy]-naphthalen-2-yl ester (0.200 mg, 0.38 mmol), palladium(II) acetate (13 mg, 0.05 mmol) and tricyclohexylphosphine (20 mg, 0.07 mmol), cesium fluoride (0.172 g, 1.13 mmol) and acetonitrile (5 mL). Heat mixture in a 90° C. oil bath for 1 hour. Cool reaction to room temperature and filter through celite and wash celite pad with ethyl acetate (60 mL). Concentrate the filtrate and pre-adsorb the crude product onto silica gel. Chromatograph the residue on a SiO₂ column eluting the material with methanol in dichloromethane (0 to 15%) to give 110 mg of the title compound (57%).

### EXAMPLE 13

### 6-{6-Methoxy-1-[4-(2-piperidin-1-yl-ethoxy)-phenoxy]-naphthalen-2-yl}-2,3-dihydro-isoindol-1-one Hydrochloride

Prepare 112 mg of the title compound from 6-{6-methoxy-1-[4-(2-piperidin-1-yl-ethoxy)-phenoxy]-naphthalen-2-yl}-2,3-dihydro-isoindol-1-one (110 mg, 0.21 mmol) as described for the preparation of 5-{6-methoxy-1-[4-(2-piperidin-1-yl-ethoxy)-phenoxy]-naphthalen-2-yl}-2,3-dihydro-isoindol-1-one hydrochloride (94%).

### EXAMPLE 14

### 6-{6-Hydroxy-1-[4-(2-piperidin-1-yl-ethoxy)-phenoxy]-naphthalen-2-yl}-2,3-dihydro-isoindol-1-one Hydrochloride

Dissolve 6-{6-methoxy-1-[4-(2-piperidin-1-yl-ethoxy)-phenoxy]-naphthalen-2-yl}-2,3-dihydro-isoindol-1-one hydrochloride (112 mg, 0.2 mmol) in dichloromethane (6 mL) and cool to 0° C. in an ice-bath. Treat solution with 1M boron tribromide in dichloromethane (0.8 mL, 0.8 mmol), drop wise over 5 minutes and stir for 45 minutes at 0° C. Add saturated aqueous sodium bicarbonate solution (2 mL) at 0° C. and warm to room temperature. Separate the resulting layers and extract the aqueous layer with ethyl acetate (5×10 mL). Wash the combined organic layers with brine, dry (Na₂SO₄) and filter. Concentrate the filtrate and pre-adsorb the crude product onto silica gel. Chromatograph the residue on a SiO₂ column eluting with methanol in dichloromethane (0 to 40%) to give 44 mg of 6-{6-hydroxy-1-[4-(2-piperidin-1-yl-ethoxy)-phenoxy]-naphthalen-2-yl}-2,3-dihydro-isoindol-1-one. Dissolve the free-base in dichloromethane (10 mL) and treat with 1M HCl in diethyl ether (10 mL, 10 mmol). Concentrate in vacuo to obtain 43 mg of the title compound (41%): mass spectrum (ion spray): m/z=495.2 (M+H—HCl).

### Preparation 6

### Mixture of 5-(4,4,5,5-Tetramethyl-[1,3,2]dioxaborolan-2-yl)-3H-isobenzofuran-1-one and 6-(4,4,5,5-tetramethyl-[1,3,2]dioxaborolan-2-yl)-3H-isobenzofuran-1-one

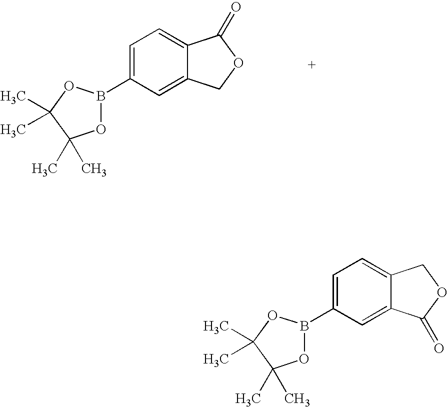

To a stirring room temperature solution of 4-bromophthalic anhydride (3.00 g, 13.22 mmol) in ethanol (10 mL) and tetrahydrofuran (50 mL), under a blanket of nitrogen, add sodium borohydride (1.96 g, 52.86 mmol), in portions. Stir this mixture at ambient temperature for 8 hours and then quench with 2N HCl (12 mL) and then excess water.

Extract the resulting aqueous mixture with diethyl ether then ethyl acetate. Wash the combined extracts with water and brine; dry (sodium sulfate) and concentrate them in vacuo to give a mixture of 5-bromo-3H-isobenzofuran-1-one and 6-bromo-3H-isobenzofuran-1-one, 2.78 g (98%). Use as is without purification.

Place the mixture of 5-bromo-3H-isobenzofuran-1-one and 6-bromo-3H-isobenzofuran-1-one (1.50 g, 7.04 mmol), bis(pinacolato)diboron (2.06 g, 8.10 mmol), PdCl₂(dppf)₂.CH₂Cl₂ (180 mg, 0.246 mmol), potassium acetate (2.07 g, 21.13 mmol) and anhydrous dimethyl sulfoxide (22 mL) in a round bottom flask. Put the reaction in an oil bath and stir it at 85° C. for 8 hours. Cool the dark brown colored reaction to ambient temperature, quench with ample water and extract the resulting aqueous mixture with dichloromethane. Wash the combined extracts with water and brine; then dry (sodium sulfate) and evaporate them in vacuo. Purify the resulting dark solid on a flash column (silica gel; 0%-20% gradient of THF in CH₂Cl₂ then 5% MeOH/20% THF/CH₂Cl₂) to provide the product as a mixture of the two title components, 785 mg (43%).

### Alternative Procedure For the Preparation of 5-(4,4,5,5-Tetramethyl-[1,3,2]dioxaborolan-2-yl)-3H-isobenzofuran-1-one

Combine 5-bromo-3H-isobenzofuran-1-one (1.0 g, 4.7 mmol), bis-pinocalatodiboron (1.8 g, 7.0 mmol), [1,1′-bis(diphenylphosphino)-ferocene]dichloropaladium(II) complex with dichloromethane (188 mg, 0.2 mmol) and potassium acetate (1.4 g, 14.0 mmol) in a 100 mL flask with a septum. Add dimethyl sulfoxide (25 mL) and heat in a 90° C. oil bath for 4 hours. Cool the resulting slurry to room temperature and dilute with water (100 mL). Extract the resulting slurry with dichloromethane (6×50 mL). Wash the combined organic layers with brine (40 mL), dry (Na₂SO₄), filter and concentrate in vacuo to obtain 1.3 g of a mixture of the title product and bis-pinocalatodiboron (1:0.06), which is used without further purification.

### EXAMPLE 15

### Mixture of 5-{6-Benzyloxy-1-[4-(2-piperidin-1-yl-ethoxy)-phenoxy]-naphthalen-2-yl}-3H-isobenzofuran-1-one and 6-{6-Benzyloxy-1-[4-(2-piperidin-1-yl-ethoxy)-phenoxy]-naphthalen-2-yl}-3H-isobenzofuran-1-one

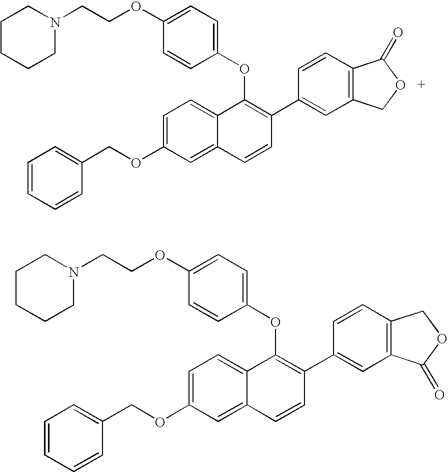

In a round bottom flask add trifluoromethanesulfonic acid 6-benzyloxy-1-[4-(2-piperidin-1-yl-ethoxy)-phenoxy]-naphthalen-2-yl ester) (592 mg, 0.984 mmol), the mixture of 5-(4,4,5,5-tetramethyl-[1,3,2]dioxaborolan-2-yl)-3H-isobenzofuran-1-one and 6-(4,4,5,5-tetramethyl-[1,3,2]dioxaborolan-2-yl)-3H-isobenzofuran-1-one (0.640 g, 2.46 mmol), a sonicated suspension of Palladium(II) Acetate (0.049 g, 0.220 mmol) and Tricyclohexylphosphine (0.091 g, 0.320 mmol) in acetonitrile (4 mL), and cesium fluoride (1.35 g, 8.86 mmol). Add acetonitrile (25 mL) and immediately place the reaction in a preheated oil bath at 90° C., and stir for 25 minutes. Then cool the reaction to ambient temperature and filter it through a pad of Celite (rinse with ample, hot ethyl acetate). Wash the filtrate with 50% aqueous sodium carbonate, saturated aqueous ammonium chloride, water and brine; then dry (sodium sulfate) and evaporate it in vacuo. Purify the resulting brown solid foam on a flash column (silica gel; 4%-10% MeOH gradient in CH₂Cl₂).

### EXAMPLE 16

### 5-{6-Benzyloxy-1-[4-(2-piperidin-1-yl-ethoxy)-phenoxy]-naphthalen-2-yl}-3H-isobenzofuran-1-one

Split the mixture of 5-{6-benzyloxy-1-[4-(2-piperidin-1-yl-ethoxy)-phenoxy]-naphthalen-2-yl}-3H-isobenzofuran-1-one and 6-{6-benzyloxy-1-[4-(2-piperidin-1-yl-ethoxy)-phenoxy]-naphthalen-2-yl}-3H-isobenzofuran-1-one into three portions and purify each on a Chromatotron (silica gel; 4%-10% MeOH gradient in EtOAc) to obtain the title compound, 0.121 g (21%): MS (IS+) m/e 586 (M+H)⁺.

### EXAMPLE 17

### 6-{6-Benzyloxy-1-[4-(2-piperidin-1-yl-ethoxy)-phenoxy]-naphthalen-2-yl}-3H-isobenzofuran-1-one

Split the mixture of 5-{6-benzyloxy-1-[4-(2-piperidin-1-yl-ethoxy)-phenoxy]-naphthalen-2-yl}-3H-isobenzofuran-1-one and 6-{6-benzyloxy-1-[4-(2-piperidin-1-yl-ethoxy)-phenoxy]-naphthalen-2-yl}-3H-isobenzofuran-1-one into three portions and purify each on a Chromatotron (silica gel; 4%-10% MeOH gradient in EtOAc) to obtain the title compound, 0.185 g (32%): MS (IS+) m/e 586 (M+H)⁺.

### EXAMPLE 18

### 5-{6-Hydroxy-1-[4-(2-piperidin-1-yl-ethoxy)-phenoxy]-naphthalen-2-yl}-3H-isobenzofuran-1-one hydrochloride

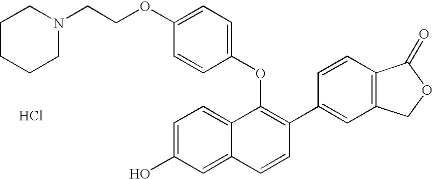

To a round bottom flask add 5-{6-benzyloxy-1-[4-(2-piperidin-1-yl-ethoxy)-phenoxy]-naphthalen-2-yl}-3H-isobenzofuran-1-one (0.111 g, 0.190 mmol), ammonium formate (0.090 g, 1.42 mmol), 10% Pd/C (0.017 g, ˜15% by weight) and MeOH (12 mL). Heat the mixture at reflux for 30 minutes. Cool the reaction to ambient temperature and filter it through a pad of Celite, then rinse the Celite with ample hot methanol. Evaporate the filtrate in vacuo and purify the resulting residue by radial chromatography over silica (5%-10% MeOH gradient in CH₂Cl₂) to provide the product free base, 62 mg. Dissolve the purified material in CH₂Cl₂ (1.5 mL) and MeOH (1.5 mL) and add 0.252 mL (2 eq) of a 1.0M solution of hydrochloric acid in diethyl ether. Shake this solution for 1 minute at ambient temperature and evaporate it in vacuo to provide the title compound, 67 mg (66%). MS (IS+) m/e 496 (M+H—HCl)⁺.

### Alternative Procedure For Preparing 5-{6-Hydroxy-1-[4-(2-piperidin-1-yl-ethoxy)-phenoxy]-naphthalen-2-yl}-3H-isobenzofuran-1-one hydrochloride

Combine trifluoro-methanesulfonic acid 6-methoxy-1-[4-(2-piperidin-1-yl-ethoxy)-phenoxy]-naphthalen-2-yl ester (1.2 mg, 2.4 mmol), 5-(4,4,5,5-Tetramethyl-[1,3,2]dioxaborolan-2-yl)-3H-isobenzofuran-1-one (1.2 g, 4.7 mmol) and acetonitrile (20 mL) in a 100 mL flask with septum. In a separate flask combine palladium(II) acetate (106 mg, 0.5 mmol) and tricyclohexylphisphine (199 mg, 0.7 mmol). Add acetonitrile (5 mL) and sonicate for 10 minutes under nitrogen. Add the catalyst slurry and cesium fluoride (3.2 g, 21.2 mmol) to mixture of substances and heat in a 78° C. oil bath for 6.5 hours. Cool the resulting suspension to room temperature and filter through packed celite. Rinse the celite with ethyl acetate. Concentrate the filtrate and pre-adsorb the crude product onto silica gel. Chromatograph the residue on a SiO₂ column eluting the material with methanol in dichloromethane (0 to 25%) to give 514 mg of 5-{6-methoxy-1-[4-(2-piperidin-1-yl-ethoxy)-phenoxy]-naphthalen-2-yl}-3H-isobenzofuran-1-one (43%).

Dissolve 5-{6-methoxy-1-[4-(2-piperidin-1-yl-ethoxy)-phenoxy]-naphthalen-2-yl}-3H-isobenzofuran-1-one (514 mg, 1.0 mmol) in dichloromethane (5 mL), treat the resulting solution with 1M HCl in diethyl ether (20 mL, 20 mmol) and concentrate the resulting suspension in vacuo to obtain 551 mg of 5-{6-methoxy-1-[4-(2-piperidin-1-yl-ethoxy)-phenoxy]-naphthalen-2-yl}-3H-isobenzofuran-1-one hydrochloride (>99%).

Dissolve 5-{6-methoxy-1-[4-(2-piperidin-1-yl-ethoxy)-phenoxy]-naphthalen-2-yl}-3H-isobenzofuran-1-one hydrochloride (551 mg, 1.0 mmol) in dichloromethane (15 mL) and cool to 0° C. in an ice-bath. Treat solution with 1M boron tribromide in dichloromethane (4.0 mL, 4.0 mmol), drop wise over 5 minutes and stir for 2.5 hours at 0° C. Add saturated aqueous sodium bicarbonate solution (12 mL) at 0° C. and warm to room temperature. Separate the resulting layers and extract the aqueous layer with ethyl acetate (5×20 mL). Wash the combined organic layers with brine, dry (Na₂SO₄) and filter. Concentrate the filtrate and pre-adsorb the crude product onto silica gel. Chromatograph the residue on a SiO₂ column eluting with methanol in dichloromethane (0 to 20%) to give 245 mg of 5-{6-Hydroxy-1-[4-(2-piperidin-1-yl-ethoxy)-phenoxy]-naphthalen-2-yl}-3H-isobenzofuran-1-one. Dissolve the free-base in dichloromethane (10 mL) and treat with 1M HCl in diethyl ether (20 mL, 10 mmol). Concentrate in vacuo to obtain 235 mg of the title compound (44%): mass spectrum (ion spray): m/z=496.3 (M+H—HCl).

### EXAMPLE 19

### 6-{6-Hydroxy-1-[4-(2-piperidin-1-yl-ethoxy)-phenoxy]-naphthalen-2-yl}-3H-isobenzofuran-1-one hydrochloride salt

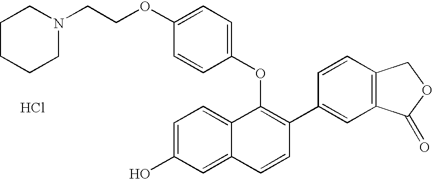

To a round bottom flask add 6-{6-benzyloxy-1-[4-(2-piperidin-1-yl-ethoxy)-phenoxy]-naphthalen-2-yl}-3H-isobenzofuran-1-one (0.156 g, 0.267 mmol), ammonium formate (0.126 g, 2.00 mmol), 10% Pd/C (0.024 g, ˜15% by weight) and MeOH (12 mL). Heat the mixture at reflux for 30 minutes. Cool the reaction to ambient temperature and filter it through a pad of Celite, then rinse the Celite with ample hot methanol. Evaporate the filtrate in vacuo and purify the resulting residue by radial chromatography over silica (5%-10% MeOH gradient in CH₂Cl₂) to provide the product free base, 73 mg. Dissolve the purified material in CH₂Cl₂ (1.5 mL) and MeOH (1.5 mL) and add 0.300 mL (2 eq) of a 1.0M solution of hydrochloric acid in diethyl ether. Shake this solution for 1 minute at ambient temperature and evaporate it in vacuo to provide the title compound, 79 mg (56%). MS (IS+) m/e 496 (M+H—HCl)⁺.

### Preparation 7

### 5-Bromo-2-methyl-isoindole-1,3-dione

To a stirring room temperature solution of 4-bromophthalimide (1.02 g, 4.51 mmol) in dimethylformamide (20 mL) add 60% sodium hydride (0.235 g, 5.87 mmol). Stir this mixture at ambient temperature for 15 minutes and then add iodomethane (0.628 mL, 10.09 mmol). Stir the reaction for 30 minutes at ambient temperature then quench it with brine. Extract the resulting aqueous mixture with ethyl acetate. Wash the combined extracts with brine; dry (sodium sulfate) and concentrate them in vacuo. Purify the resulting material on a flash column (silica gel; 70%-100% CH₂Cl₂ gradient in hexanes) to obtain the title compound, 0.960 g (89%). MS (IS+) m/e 240 (M+H, ⁷⁹Br), 242 (M+H, ⁸¹Br).

### Preparation 8

### 2-Methyl-5-(4,4,5,5-tetramethyl-[1,3,2]dioxaborolan-2-yl)-isoindole-1,3-dione

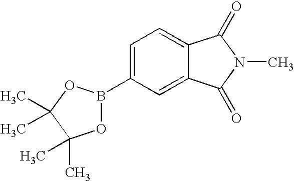

Place 5-bromo-2-methyl-isoindole-1,3-dio 6 (0.920 g, 3.83 mmol), Bis(pinacolato)diboron (1.07 g, 4.21 mmol), PdCl₂(dppf)₂.CH₂Cl₂ (0.098 g, 0.134 mmol), potassium acetate (1.13 g, 11.49 mmol) and anhydrous dimethyl sulfoxide (12 mL) in a round bottom flask. Put the reaction in an oil bath and stir it at 85° C. for 8 hours. Cool the dark brown colored reaction to ambient temperature, quench with ample water and extract the resulting aqueous mixture with dichloromethane. Wash the combined extracts with water and brine; then dry (sodium sulfate) and evaporate them in vacuo. Purify the resulting dark solid on a flash column (silica gel; 2%-15% gradient of EtOAc in CH₂Cl₂) to provide the title compound, 0.67 g (61%). MS (IS−) m/e 204 (M−H—pinacol ester)⁻, (IS+) m/e 288 (M+H)⁺.

### EXAMPLE 20

### 5-{6-Benzyloxy-1-[4-(2-piperidin-1-yl-ethoxy)-phenoxy]-naphthalen-2-yl}-2-methyl-isoindole-1,3-dione

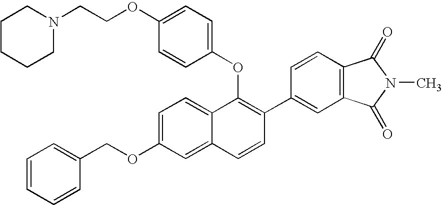

In a round bottom flask add trifluoromethanesulfonic acid 6-benzyloxy-1-[4-(2-piperidin-1-yl-ethoxy)-phenoxy]-naphthalen-2-yl ester (0.200 g, 0.332 mmol), 2-methyl-5-(4,4,5,5-tetramethyl-[1,3,2]dioxaborolan-2-yl)-isoindole-1,3-dione (0.239 g, 0.831 mmol), Palladium(II) Acetate (0.015 g, 0.066 mmol) and tricyclohexylphosphine (0.028 g, 0.099 mmol). Add cesium fluoride (0.454 g, 2.99 mmol) and immediately add acetonitrile (12 mL). Place the reaction in a preheated oil bath at 90° C., and stir for 25 minutes. Then cool the reaction to ambient temperature and filter it through a pad of Celite (rinse with ample, hot ethyl acetate). Wash the filtrate with 50% aqueous sodium carbonate, saturated aqueous ammonium chloride, water and brine; then dry (sodium sulfate) and evaporate it in vacuo. Purify the resulting tan solid on a Chromatotron (silica gel; 2%-8% MeOH gradient in CH₂Cl₂) to obtain the title compound, 0.154 g (76%). MS (IS+) m/e 613 (M+H)⁺.

### EXAMPLE 21

### 5-{6-Hydroxy-1-[4-(2-piperidin-1-yl-ethoxy)-phenoxy]-naphthalen-2-yl}-2-methyl-isoindole-1,3-dione hydrochloride salt

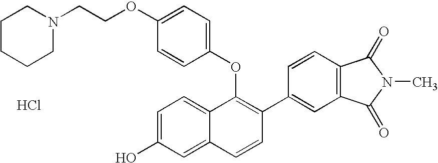

To a round bottom flask add 5-{6-benzyloxy-1-[4-(2-piperidin-1-yl-ethoxy)-phenoxy]-naphthalen-2-yl}-2-methyl-isoindole-1,3-dione (0.136 g, 0.222 mmol), ammonium formate (0.105 g, 1.67 mmol), 10% Pd/C (0.020 g, ˜15% by weight) and MeOH (7.5 mL). Heat the mixture at reflux for 30 minutes. Cool the reaction to ambient temperature and filter it through a pad of Celite, then rinse the Celite with ample hot methanol. Evaporate the filtrate in vacuo and purify the resulting residue by radial chromatography over silica (5%-12% MeOH gradient in CH₂Cl₂) to provide the product free base, 75 mg. Dissolve the purified material in CH₂Cl₂ (2 mL) and MeOH (2 mL) and add 0.287 mL (2 eq) of a 1.0M solution of hydrochloric acid in diethyl ether. Shake this solution for 1-2 minutes at ambient temperature and evaporate it in vacuo to provide the title compound, 80 mg (64%). MS (IS+) m/e 523 (M+H—HCl)⁺.

### EXAMPLE 22

### 4-{6-methoxy-1-[4-(2-piperidin-1-yl-ethoxy)-phenoxy]-naphthalen-2-yl}-N-methyl-benzamide hydrochloride

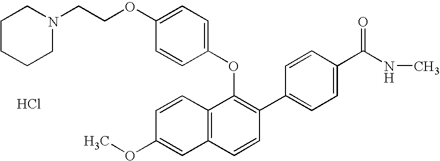

Combine trifluoromethanesulfonic acid 6-methoxy-1-[4-(2-piperidin-1-yl-ethoxy)-phenoxy]-naphthalen-2-yl ester (200 mg, 0.38 mmol), N-Methyl-4-(4,4,5,5-tetramethyl-[[1,3,2]dioxaborolan-2-yl)-benzamide (300 mg, 1.14 mmol), [1,1′-bis(diphenylphosphino)-ferrocene)dichloropalladium (II), complex with dichloromethane (1:1) (300 mg, 0.38 mmol), cesium fluoride (500 mg, 3.43 mmol) and acetonitrile (4 mL), stir and heat at 85° C. After 18 h, cool to ambient temperature and filter through celite. The crude reaction mixture is purified using radial chromatography eluting with 6% methanol in dichloromethane, combining product fractions to 100 mg (34%) of a brown oil. The hydrochloride salt is formed by adding 0.8mL of a 1N HCl in Et₂O solution and dried to give 110 mg of a tan solid which is used without further purification. Mass spectrum (ion spray): m/z=511(M+1).

### EXAMPLE 23

### 4-{6-hydroxy-1-[4-(2-piperidin-1-yl-ethoxy)-phenoxy]-naphthalen-2-yl}-N-methyl-benzamide hydrochloride

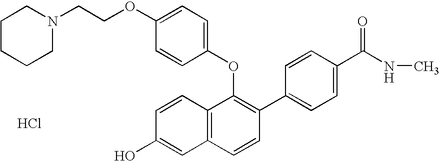

Charge a 100 mL round-bottom flask with 4-{6-methoxy-1-[4-(2-piperidin-1-yl-ethoxy)-phenoxy]-naphthalen-2-yl}-N-methyl-benzamide hydrochloride (110 mg, 0.20 mmol) and cool to 0° C. under nitrogen. Add 0.6 mL of a 1M CH₂Cl₂ solution of BBr₃ and monitor the reaction by ES-MS. After stirring for 1 hour, add an additional 0.6 mL of a 1M CH₂Cl₂ solution of BBr₃. After stirring an additional hour, pour the reaction into a cold saturated solution of aqueous sodium bicarbonate and ethyl acetate (150 mL). Dry the organic layer is dried over sodium sulfate and concentrate in vacuo. The crude product is purified by radial chromatography to yield 62 mg (62%) of the free base of the title compound. Form the hydrochloride salt by adding 0.8 mL of a 1N HCl in Et₂O solution to give 73 mg of the title compound. Mass spectrum (ion spray): m/z=497(M+1). HRMS calcd for C₃₁H₃₃N₂O₄ (M+H): 497.2440. Found 497.2444.

### EXAMPLE 24

### 4-{6-Hydroxy-1-[4-(2-piperidin-1-yl-ethoxy)-phenoxy]-naphthalen-2-yl}-N,N-dimethyl-benzamide hydrochloride

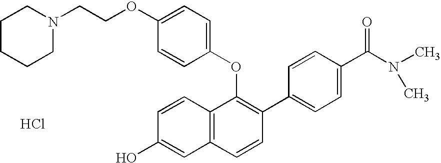

Combine trifluoromethanesulfonic acid 6-methoxy-1-[4-(2-piperidin-1-yl-ethoxy)-phenoxy]-naphthalen-2-yl ester (247 mg, 0.47 mmol), 4-(N,N-Dimethylaminocarbonyl)phenylboronic acid (272 mg, 1.41 mmol), palladium acetate (II), (32 mg, 0.14 mmol), cesium fluoride (643 mg, 4.23 mmol), tricyclohexylphosphine (43 mg, 0.16 mmol) and acetonitrile (7 mL), stir and heat at 90° C. After 90 min, cool to ambient temperature and filter through celite. Purify the crude reaction mixture using radial chromatography eluting with 4% methanol in dichloromethane, combining product fractions to give 256 mg of 4-{6-methoxy-1-[4-(2-piperidin-1-yl-ethoxy)-phenoxy]-naphthalen-2-yl}-N,N-dimethyl-benzamide. Form the hydrochloride salt by adding 0.8 mL of a 1N HCl in Et₂O solution and dry to give 264 mg of the corresponding hydrochloride salt.

Prepare the title compound in a manner analogous to that of 5-{6-hydroxy-1-[4-(2-piperidin-1-yl-ethoxy)-phenoxy]-naphthalen-2-yl}-2,3-dihydro-isoindol-1-one hydrochloride using 4-{6-methoxy-1-[4-(2-piperidin-1-yl-ethoxy)-phenoxy]-naphthalen-2-yl}-N,N-dimethyl-benzamide hydrochloride (264 mg, 0.47 mmol). Purify the crude product by radial chromatography to yield 122 mg (51%) of the free base of title compound. Mass spectrum (ion spray): m/z=511(M+1). Form the hydrochloride salt by adding 0.7 mL of a 1N HCl in Et₂O solution to give 131 mg of the title compound.

### Preparation 9

### 2,4-dibenzyloxyphenyl boronic acid

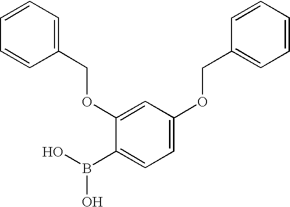

Dissolve 4-bromo-resorcinol 25.0 g (0.132 mol) in 250 mL of DMF. Add K₂CO₃ 45.0 g (0.31 mol). Add benzylbromide 32.0 mL (0.27 mol) drop wise with vigorous stirring. Heat the reaction to 100° C. until TLC shows no starting phenol (3 to 5 hours). After an aqueous workup, purify the product by flash chromatography on silica gel using 10% ethyl acetate in hexane as eluent. Remove the solvent to give 41.0 g of 4-bromo-resorcinol dibenzyl ether (84%).

Dissolve 4-bromo-resorcinol dibenzyl ether (41.0 g, 0.11 mol) in 200 mL of THF. Add butyllithium (1.6 M in THF) 75.0 mL (0.12 mol) dropwise via syringe at −78° C. with vigorous stirring. Stir the reaction for another hour to ensure complete reaction. Add triethylborate 20 mL (0.14 mol) all at once. Allow the reaction to warm to room temperature overnight. Pour the reaction mixture into 500 mL of water and 200 mL of ethyl acetate. Separate the layers. Carefully adjust the aqueous phase to pH 7˜8 with saturated NH₄Cl and extract with ethyl acetate. Wash the combined organic with brine and dry over MgSO₄. Evaporate the solvent to give the title compound.

### Preparation 10

### Trifluoromethanesulfonic acid 2-methoxy-5-[4-(2-piperidin-1-yl-ethoxy)-phenyl]-5H-6-oxa-chrysen-8-yl ester

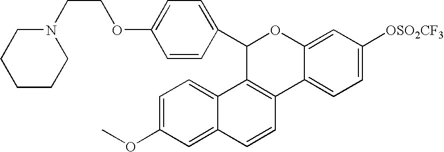

Dissolve 2,6-dimethoxynaphthalene 37.6 g (0.20 mol) and 4-(2-(piperidin-1-yl)ethoxy)benzoyl chloride 64.0 g (0.21 mol) in 800 mL of dichloromethane. Add aluminum chloride 133 g (1.00 mol) portionwise and slowly (the first 30 to 50 g must be added slowly to keep the acylation reaction under control so the solvent does not boil off). After all the aluminum chloride has been added, stir the reaction until no more undemethylated compound can be detected either by TLC or HPLC (about 5 hours). Slowly pour the reaction mixture into 1 L of ice/water with vigorous stirring. Decant the top layer water into a separation funnel. Wash the dichloromethane solution and the precipitate with 2N HCl and decant the aqueous layer again into the separation funnel. Extract the aqueous layer with dichloromethane. Adjust the combined dichloromethane solution and the precipitate pH to 8 first with 1N NaOH then with saturated NaHCO₃. Filter the mixture. Slurry the solid repeatedly with dichloromethane. Separate the layers of the filtrate and extract the aqueous phase with dichloromethane. Wash the combined organic with brine and dry over MgSO₄. Treat the dichloromethane solution with charcoal and filter through a prepackaged “suppelco” silica gel funnel. Evaporate the solvent to give 61.2 g (75.5%) of 6-methoxy-1-[4-(2-piperidin-1-yl-ethoxy)-benzoyl]-naphthalen-2-ol.

Couple 6-methoxy-1-[4-(2-piperidin-1-yl-ethoxy)-benzoyl]-naphthalen-2-ol and 2,4-dibenzyloxyphenyl boronic acid to provide 2-(2,4-dibenzyloxyphenyl)-6-methoxy-1-[4-(2-piperidin-1-yl-ethoxy)-benzoyl]-naphthalene by the procedure analogous to that described above in the procedure for 5-{6-benzyloxy-1-[4-(2-piperidin-1-yl-ethoxy)-phenoxy]-naphthalen-2-yl}-2-methyl-isoindole-1,3-dione.

Dissolve 10.5 g (20.0 mmol) 2-(2,4-dibenzyloxyphenyl)-6-methoxy-1-[4-(2-piperidin-1-yl-ethoxy)-benzoyl]-naphthalene in 150 mL of THF. Add LAH 1.5 g (37.0 mmol) portionwise with vigorous stirring at 0° C. After the addition, allow the reaction to warm up to room temperature and then stir for 3 hours. Cool the reaction in an ice bath and slowly quench with saturated Na₂SO₄. Filter off the solid Al₂O₃ and wash the filter cake with THF (2×50mL). Combine the filtrates, concentrate and purify the residue by flash chromatography on silica gel using CH₂Cl₂:MeOH (9:1) as eluent to afford 2-methoxy-5-{hydroxy-[4-(2-piperidin-1-yl-ethoxy)-phenyl]-methyl}-6-(2,4-benzyloxyphenyl)-naphthalene.

Heat 2-methoxy-5-{hydroxy-[4-(2-piperidin-1-yl-ethoxy)-phenyl]-methyl}-6-(2,4-benzyloxyphenyl)-naphthalene to 60° C. in THF containg 10% (by weight) of Pd/C (30%) catalyst, overnight under 50 psi of hydrogen atmosphere to afford 2-methoxy-5-{hydroxy-[4-(2-piperidin-1-yl-ethoxy)-phenyl]-methyl}-6-(2-hydroxy-4-benzyloxyphenyl)-naphthalene. Treat the THF solution of 2-methoxy-5-{hydroxy-[4-(2-piperidin-1-yl-ethoxy)-phenyl]-methyl}-6-(2-hydroxy-4-benzyloxyphenyl)-naphthalene with 10% (by mol) of concentrated HCl to give 1-{2-[4-(8-benzyloxy-2-methoxy-5H-6-oxa-chrysen-5-yl)-phenoxy]-ethyl}-piperidine.

Dissolve 1-{2-[4-(8-benzyloxy-2-methoxy-5H-6-oxa-chrysen-5-yl)-phenoxy]-ethyl}-piperidine (680 mg) in a mixture of 250 ml ethanol and 150 ml THF with warming. Add a slurry of 300 mf 10% Pd/C in ethanol and react under 1 atmosphere of hydrogen for 18 hours. Filter the catalyst and evaporate the solvent to yield 465 mg of 2-methoxy-5-[4-(2-piperidin-1-yl-ethoxy)-phenyl]-5H-6-oxa-chrysen-8-ol.

Dissolve 2-methoxy-5-[4-(2-piperidin-1-yl-ethoxy)-phenyl]-5H-6-oxa-chrysen-8-ol (118 mg., 0.245 mmoles) in 20 ml methylene chloride and add N-phenyltrifluoromethanesulfonimide (400 mg,. 1.12 mmoles) followed by 1.0 ml of diisopropylethyl amine and stir for 72 hours. Evaporate the solution to a paste and purify by running through an SCX column in methanol (elute with 2N ammonia/methanol) to give 125 mg of the title compound: 125 mg (83%).

### Alternative Synthesis of Trifluoromethanesulfonic acid 2-methoxy-5-[4-(2-piperidin-1-yl-ethoxy)-phenyl]-5H-6-oxa-chrysen-8-yl ester

Dissolve trifluoromethanesulfonic acid 6-methoxy-1-[4-(2-piperidin-1-yl-ethoxy)-benzoyl]-naphthalen-2-yl ester, (1.89 grams, 3.52 mmoles) in 100 ml acetonitrile and add to a flask containing bis(pinacolato)diboron (1.07 grams, 4.23 mmoles), palladium acetate (79 mg. 0.35 mmoles), triphenylphosphine (185 mg. 0.70 mmoles) and cesium fluoride (1.6 grams, 10.56 mmoles). Heat and stir the mixture under nitrogen for two hours at reflux. Cool the reaction slightly and add 2,4 bis(benzyloxy)bromobenzene (2.6 grams, 7.0 mmoles) along with another portion of the diboron, palladium acetate, and triphenylphosphine. Continue refluxing for 24 hours. Cool the mixture, filter off the solids and run the filtrate through an SCX column. Wash the columns with methanol and elute with 2N ammonia in methanol. Evaporate the filtrate to give 1.8 grams of a dark brown gum. Purify on a flash column using silica gel eluting with a gradient of 0 to 5% methanol in methylene chloride. Evaporate the solvent to yield 1.1 gram of [2-(2,4-bis-benzyloxyphenyl)-6-methoxynaphthalen-1-yl]-[4-(2-piperidin-1-yl-ethoxy)-phenyl]-methanone (46%).

Dissolve [2-(2,4-Bis-benzyloxyphenyl)-6-methoxynaphthalen-1-yl]-[4-(2-piperidin-1-yl-ethoxy)-phenyl]methanone (1.1 grams, 1.6 mmoles) in 10 ml of tetrahydrofuran (THF) and add a 1.0 molar solution of lithium aluminum hydride (5 ml. 5.0 mmoles). Stir for 30 minutes at which time the reaction is complete as determined by LC/MS. Quench the reaction with sodium bicarbonate solution and extract with a 3/1 mixture of chloroform and isopropanol. Acidify the water layer to pH=7.0 and extract again. Combine the organic layers and dry over 3A molecular sieves. Evaporate the solvent to give 1.0 g of [2-(2,4-bis-benzyloxy-phenyl)-6-methoxynaphthalen-1-yl]-[4-(2-piperidin-1-yl-ethoxy)-phenyl]-methanol.

Dissolve [2-(2,4-bis-benzyloxy-phenyl)-6-methoxynaphthalen-1-yl]-[4-(2-piperidin-1-yl-ethoxy)-phenyl]-methanol (900 mg., 1.32 mmoles) in 250 ml of THF and add 20 ml of 5N HCl and 700 mg 10% Pd/C (slurried in THF). Place the reaction mixture under a balloon of nitrogen and stir for 24 hours. Filter the reaction mixture and add saturated sodium bicarbonate. Extract the aqueous phase 2 times with a 3/1 mixture of chloroform and isopropanol. Dry the organic layer over 3A molecular sieves, evaporate and triturate the resulting gum with ether to give 521 mg of 2-methoxy-5-[4-(2-piperidin-1-yl-ethoxy)-phenyl]-5H-6-oxa-chrysen-8-ol (82%).

Dissolve 2-methoxy-5-[4-(2-piperidin-1-yl-ethoxy)-phenyl]-5H-6-oxa-chrysen-8-ol (96 mg. 0.2 mmoles) in 10 ml. of methylene chloride and add N,N-bis(trifluoromethylsulfonyl)aniline (92 mg. 0.25 mmoles) followed by diisopropylethyl amine (32 mg., 0.25 mmoles). Stir for 2 hours and check by LC/MS. Still much starting material left so another portion of the aniline and amine are added. After 2 hours still considerable starting material left, so add 500 microliters of the amine and leave stand overnight. In the morning the reaction is complete. Rotavap the solvent, dissolve the residue in methanol and run through and SCX column, eluting with 2N ammonia in methanol. Evaporate the solvent to give 74 mg of the title compound (61%).

### EXAMPLE 25

### 2-Methoxy-5-[4-(2-piperidin-1-yl-ethoxy)-phenyl]-5H-6-oxa-chrysene-8-carboxylic acid methyl ester

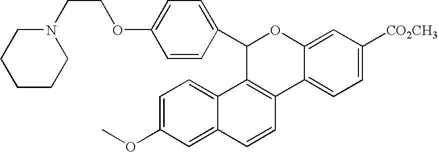

Dissolve trifluoromethanesulfonic acid 2-methoxy-5-[4-(2-piperidin-1-yl-ethoxy)-phenyl]-5H-6-oxa-chrysen-8-yl ester (180 mg.) in 25 ml DMF and add 10 ml of methanol, 0.1 ml triethylamine, palladium acetate (6.7 mg) and 1,1-bis(diphenylphosphino)ferrocene (39.2 mg). Run under carbon monoxide at 1000 psi at 100 degrees for 24 hours. Add Celite and filter, evaporating the solvent to yield a dark oil. Purify on a silica column eluting first with methylene chloride, then with 3% methanol/methylene chloride to elute the product. Evaporate the solvent to yield 52 mg (83%) of the title compound which on LC/MS has a retention time of 3.2 minutes, and a mass of 524 (M+1).

### Alternative Synthesis of 2-Methoxy-5-[4-(2-piperidin-1-yl-ethoxy)-phenyl]-5H-6-oxa-chrysene-8-carboxylic acid methyl ester

Dissolve trifluoromethanesulfonic acid 2-methoxy-5-[4-(2-piperidin-1-yl-ethoxy)-phenyl]-5H-6-oxa-chrysen-8-yl ester (110 mg 0.179 mmoles) in 25 ml of methanol and add 0.1 equivalent of palladium acetate, 0.1 equivalent of diphenylphosphinobutane and 2.2 equivalents of triethyl amine. React in a high pressure vessel with carbon monoxide at 1000 psi and 110 degrees. Purify product by running it through an SCX column and eluting with 2 N ammonia in methanol to give 46 mg of the title compound (43%).

### EXAMPLE 26

### 2-Hydroxy-5-[4-(2-piperidin-1-yl-ethoxy)-phenyl]-5H-6-oxa-chrysene-8-carboxylic acid ammonium salt

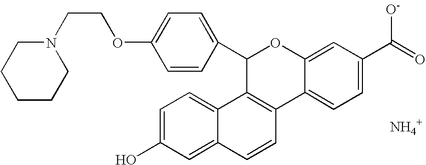

Dissolve 2-methoxy-5-[4-(2-piperidin-1-yl-ethoxy)-phenyl]-5H-6-oxa-chrysene-8-carboxylic acid methyl ester (20 mg.) in 5.0 ml ethanol and add 1 ml of 5 N sodium hydroxide. Warm on a steam bath for 5 minutes and allow to stand for 2 hours. Remove the solvents under vacuum, and add 20 ml DMF along with sodium t-butyl mercaptan. Seal the vessel, purge with nitrogen and heat at 150 degrees for 24 hours. Remove the solvent under vacuum, add methanol and acetic acid and run the mixture through an SCX column. Elute the product with 2 N ammonia/methanol. Evaporate the solvent to give 6 mg of the title compound, which on LC/MS has a retention time of 5.2 minutes, and a mass of 496 (M+1).

### EXAMPLE 27

### 2-Methoxy-5-[4-(2-piperidin-1-yl-ethoxy)-phenyl]-5H-6-oxa-chrysene-8-carboxylic acid ammonium salt

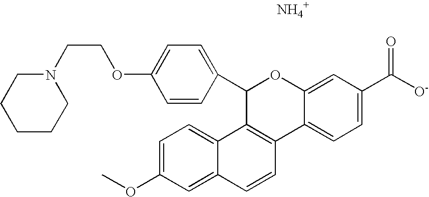

Dissolve trifluoromethanesulfonic acid 2-methoxy-5-[4-(2-piperidin-1-yl-ethoxy)-phenyl]-5H-6-oxa-chrysen-8-yl ester (23 mg. ) in 3 ml of DMSO and add 0.42 mg of palladium II acetate along with 4.2 mg of 1,1-bis(diphenylphosphino)ferrocene and 15 mg of potassium acetate. Seal the vial, purge with carbon monoxide and heat at 60 degrees for 4 hours. At this time add more of the palladium acetate and DPPF, purge with the CO and heat as before. Cool the mixture, add methanol and run through an SCX column, eluting the product with 2 N ammonia/methanol. The product contained both ester and acid, so the ester is hydrolyzed with sodium hydroxide, evaporated to dryness and used in the next step.

### EXAMPLE 28

### 2-Hydroxy-5-[4-(2-piperidin-1-yl-ethoxy)-phenyl]-5H-6-oxa-chrysene-8-carboxylic acid dimethylamide

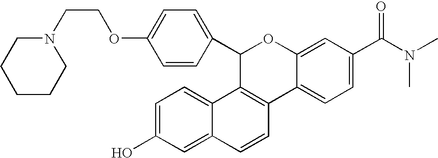

Dissolve 2-methoxy-5-[4-(2-piperidin-1-yl-ethoxy)-phenyl]-5H-6-oxa-chrysene-8-carboxylic acid ammonium salt in methylene chloride and add 2 drops of DMF followed by excess oxalyl chloride. After the bubbling stops, evaporate the solvent, add methanol and purify on an SCX column, eluting the product with 2N ammonia/methanol. Deprotect the material with the sodium salt of t-butyl mercaptan in DMF at 110 degrees for 18 hours. Purify using reverse phase chromatography to yield 1.5 mg of the title compound which on LC/MS has a retention time of 2.9 minutes and a mass of 523 (M+1).

### Preparation 11

### 2-Methoxy-5-[4-(2-piperidin-1-yl-ethoxy)-phenyl]-5H-6-oxa-chrysene-8-carbonitrile

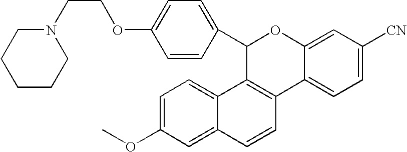

Dissolve trifluoromethanesulfonic acid 2-methoxy-5-[4-(2-piperidin-1-yl-ethoxy)-phenyl]-5H-6-oxa-chrysen-8-yl ester (100 mg., 0.163 mmoles) in 8 ml of DMF and add zinc cyanide (100 mg,. 0.85 mmoles) and palladium (0) tetrakis (triphenylphosphine) (38 mg., 0.033 mmoles). Purge the vial with nitrogen, seal and heat at 80 degrees for 1 hour.

Evaporate the DMF, add methanol, filter off and discard the solid, and run the filtrate through an SCX column, eluting the product with 2 N ammonia/methanol. Evaporate the solvent and purify on a small silica column eluting the product with 4% methanol/methylene chloride. Yield 60 mg (75%).

### EXAMPLE 29

### 2-Methoxy-5-[4-(2-piperidin-1-yl-ethoxy)-phenyl]-5H-6-oxa-chrysene-8-carboxylic acid amide

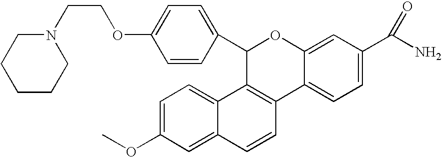

Dissolve 2-methoxy-5-[4-(2-piperidin-1-yl-ethoxy)-phenyl]-5H-6-oxa-chrysene-8-carbonitrile (36 mg.) in 10 ml DMSO and add 85 mg potassium carbonate followed by 100 micoliters of 30% hydrogen peroxide. Stir for 1 hour and add another 100 microliters of hydrogen peroxide and stir for another hour. Filter the reaction, dilute with methanol and pass through an SCX column washing with methanol and eluting the product with 2N ammonia/methanol. Evaporate to dryness to yield 25 mg of the title compound which has a retention time of 5.9 minutes and a mass of 509 (M+1) on LC/MS.

### EXAMPLE 30

### 2-Hydroxy-5-[4-(2-piperidin-1-yl-ethoxy)-phenyl]-5H-6-oxa-chrysene-8-carboxylic acid amide

Dissolve 2-methoxy-5-[4-(2-piperidin-1-yl-ethoxy)-phenyl]-5H-6-oxa-chrysene-8-carboxylic acid amide (25 mg.) in 10 ml of DMF and add a large excess of sodium t-butlylthiolate, seal the vial and heat at 110 degrees for 6 hours. Cool the reaction, add acetic acid and evaporate to an oil. Dissolve the oil in methanol, add to an SCX column, wash the column with methanol and elute the product with 2 N ammonia/methanol. Evaporate the solvent to yield the title compound that has a retention time of 3.2 minutes and a mass of 495 (M+1) on LC/MS.

### Preparation 12

### Trifluoromethanesulfonic acid 6-methoxy-1-[4-(2-piperidin-1-yl-ethoxy)-benzoyl]-naphthalen-2-yl ester

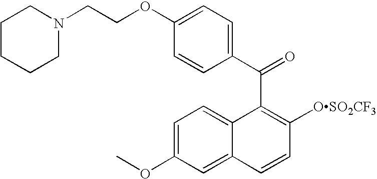

Dissolve 2,6-dimethoxynaphthalene (1.0 eq) in CH₂Cl₂ (5 volume equivalents) at ambient temperature in a dry round bottom flask equipped with stir bar, temperature probe and N₂ line. Cool the solution to 0° C. with an ice bath, and add 4-(2-piperidin-1-yl-ethoxy)-benzoyl chloride (1.1 eq). Add aluminum chloride (2.0 eq). Once the reaction is determined to be complete, quench the reaction slowly with 1 N NaOH and dilute with additional water and CH₂Cl₂. Wash the aqueous layer with CH₂Cl₂ (20 mL). Combine the organic extracts and wash with brine and dry (Na₂SO₄). Recrystallize the crude product from methanol to give (2,6-dimethoxy-naphthalen-1-yl)-[4-(2-piperidin-1-yl-ethoxy)-phenyl]-methanone.

Dissolve (2,6-dimethoxy-naphthalen-1-yl)-[4-(2-piperidin-1-yl-ethoxy)-phenyl]-methanone in CH₂Cl₂ (10 volume equivalents) in a 3-neck round bottom flask equipped with a pressure equalizing addition funnel, stirbar, and N₂ source. Cool the flask in an ice/brine bath and add 1.0 M BCl₃ solution in CH₂Cl₂ (1.2 equivalents) dropwise. The reaction solution turns dark red and the temperature initially increases to 5° C. After about 1 hour, quench the reaction with methanol (5 equivalents) and allow to warm to room temperature. Dilute the organic solution with CH₂Cl₂ (one volume equivalent) and add a 1.0 M NaHCO₃ solution (5 volume equivalents) and stir for one hour. Separate the aqueous and organic layers. Wash the aqueous layer with CH₂Cl₂ (one volume) and combine the organic layers. Wash with saturated NH₄Cl and dry over Na₂SO₄. Purify the product via column chromatography (50/1 silica gel) eluting with CH₂Cl₂/hexanes (3/1) to yield (2-hydroxy-6-methoxy-naphthalen-1-yl)-[4-(2-piperidin-1-yl-ethoxy)-phenyl]-methanone.

Dissolve (2-hydroxy-6-methoxy-naphthalen-1-yl)-[4-(2-piperidin-1-yl-ethoxy)-phenyl]-methanone in CH₂Cl₂ (10 volumes) in a three neck round bottom flask equipped with a stir bar and N₂ source and chill to 0° C. in an ice/brine bath. Add pyridine (1.3 equivalents). Add trifluoromethanesulfonyl chloride (1.2 equivalents) via syringe over 15 minutes. After about 15 minutes, quench the reaction with H₂O (10 volumes), wash with 1 N aqueous HCl (5 volumes) and 1.0 N aqueous NaHCO₃, and dry over Na₂SO₄. Obtain the title compound in quantitative yield after concentration.

### Preparation 13

### 6-Methoxy-naphthalen-1-yl)-[4-(2-piperidin-1-yl-ethoxy)-phenyl]-methanone-2-boronic acid

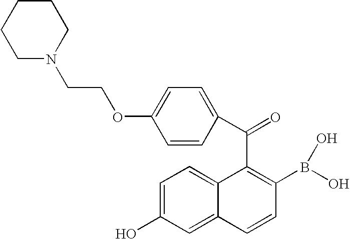

Dissolve trifluoromethanesulfonic acid 6-methoxy-1-[4-(2-piperidin-1-yl-ethoxy)-benzoyl]-naphthalen-2-yl ester (2.0 gm., 3.72 mmoles) in 125 ml methanol and heat to 55 degrees. To this add tricyclohexylphosphine (208 mg., 0.74 mmoles) followed by palladium II acetate (84 mg., 0.37 mmoles), bis(neopentyl glycolato)diboron (2.5 gm., 11.1 mmoles) and cesium fluoride (1.7 gm., 11.2 mmoles). Stir the reaction at 55 degrees for 4 hours. Cool the reaction, filter, and concentrate the filtrate to 60 ml and purify on an SCX column eluting the product with 2 N ammonia/methanol. Evaporate the solvent, then triturate with ether to give 1.1 grams (69%) of the title compound.

### Preparation 14

### 4-Hydroxy-3-{6-hydroxy-1-[4-(2-piperidin-1-yl-ethoxy)-benzoyl]-naphthalen-2-benzoic acid

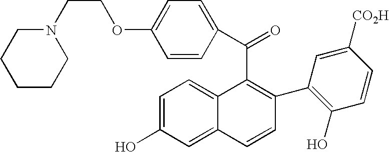

Dissolve 6-methoxy-naphthalen-1-yl)-[4-(2-piperidin-1-yl-ethoxy)-phenyl]-methanone-2-boronic acid (433 mg., 1.0 mmoles) and 3-iodo-4-methoxybenzoic acid (556 mg., 2.0 mmoles) in 8 ml of ethanol and add a slurry of 500 mg. of 10% palladium on carbon in 3 ml ethanol followed by 840 mg of sodium carbonate. Flush the vial with nitrogen and seal. Heat the mixture at 72 degrees for 24 hours. Cool, filter, wash the solid with ethanol and discard the solid. Purifty the filtrate on an SCX column, washing with methanol and eluting the product with 2N ammonia/methanol. Evaporate the solvent and purify on a silica column, eluting the impurities with a 0-10% methanol/methylene chloride gradient, then eluting the product with 20% methanol/methylene chloride to give 56 mg, 10%, of 3-methoxy-4-{6-methoxy-1-[4-(2-piperidin-1-yl-ethoxy)-benzoyl]-naphthalen-2-yl}-benzoic acid.

Convert 3-methoxy-4-{6-methoxy-1-[4-(2-piperidin-1-yl-ethoxy)-benzoyl]-naphthalen-2-yl}-benzoic acid (56 mg) to the HCl salt and dissolve in methylene chloride. Chill the solution in ice and add excess boron tribromide in portions. Stir at 0 degrees for 1 hour, then at room temperature for 1 hour. Add a few drops of boron tribromide and stir for another ½ hour. Quench the mixture with saturated sodium bicarbonate and wash the water layer with a solvent composed of a 3/1 mixture of chloroform/isopropanol. Adjust the pH of the water layer to 7 and extract with the organic solvent. Combine the organic layers, dry over 3a molecular sieves and evaporate to a solid. Purify on an SCX column, eluting with 2N ammonia/methanol to give 16 mg (30%) of the title compound.

### EXAMPLE 31

### 2-Hydroxy-5-[4-(2-piperidin-1-yl-ethoxy)-phenyl]-5H-6-oxa-chrysene-9-carboxylic acid trifluoroacetate

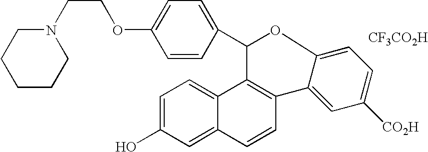

Dissolve 4-hydroxy-3-{6-hydroxy-1-[4-(2-piperidin-1-yl-ethoxy)-benzoyl]-naphthalen-2-yl}-benzoic acid (16 mg.) in 10 ml methylene chloride and add 1.0 ml of trifluoroacetic acid followed by 1.0 ml of triethylsilane. Stir for 1 hour and quench with sodium bicarbonate solution. Extract the water with a 3/1 mixture of chloroform/isopropanol, adjust the water layer to a pH of 7 and extract again. Combine the organic layers, dry over 3A sieves, evaporate and purify by reverse phase HPLC using trifluoroacetic acid in the chromatography solvent to give 7.8 mg (50%) of the title compound. Parent ion of 495 on MS.

### EXAMPLE 32

### 2-Methoxy-5-[4-(2-piperidin-1-yl-ethoxy)-phenyl]-5H-6-oxa-chrysene-8-carboxylic acid methylamine salt

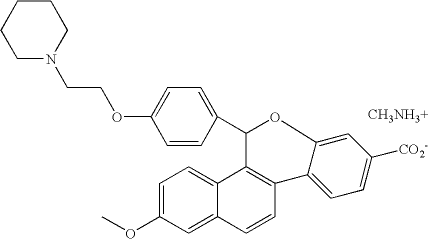

Dissolve 2-methoxy-5-[4-(2-piperidin-1-yl-ethoxy)-phenyl]-5H-6-oxa-chrysene-8-carboxylic acid methyl ester (69 mg.) in ethanol and add 1.0 ml of 1 N sodium hydroxide. Warm until all is in solution and let stand overnight. Neutralize with 1 N HCl and add to an SCX column. Elute the product with 2 N methylamine/methanol. Evaporate to dryness, which yields a product with the correct mass. Take the material on to the next step without further purification.

### EXAMPLE 33

### 2-Methoxy-5-[4-(2-piperidin-1-yl-ethoxy)-phenyl]-5H-6-oxa-chrysene-8-carboxylic acid methylamide hydrochloride

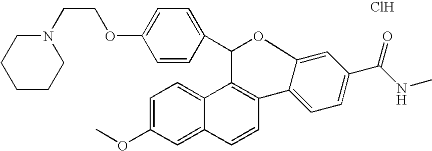

Dissolve 2-methoxy-5-[4-(2-piperidin-1-yl-ethoxy)-phenyl]-5H-6-oxa-chrysene-8-carboxylic acid methylamine salt in methylene chloride and add a large excess of oxalyl chloride with stirring. Stir the reaction for 1 hour, and evaporate to dryness. Add methylene chloride and methylamine in THF solution and stir one hour. Add the mixture to an SCX column, wash with methanol and elute the product with 2N ammonia/methanol. The title compound has a retention time of 4.6 minutes and a mass of 523 (M+1) on LC/MS. The compound is converted to the HCl salt and lyophilized giving 60 mg. of product.

### EXAMPLE 34

### 2-Hydroxy-5-[4-(2-piperidin-1-yl-ethoxy)-phenyl]-5H-6-oxa-chrysene-8-carboxylic acid methylamide hydrochloride

Dissolve 2-methoxy-5-[4-(2-piperidin-1-yl-ethoxy)-phenyl]-5H-6-oxa-chrysene-8-carboxylic acid methylamide (56 mg) in 10 ml DMF and add a large excess of sodium t-butylthiolate. Seal the vial and heat at 110 degrees for 48 hours. Cool the mixture, add acetic acid and evaporate to ½ the original volume. Add methanol and run through an SCX column washing with methanol and eluting the product with 2N ammonia/methanol. Evaporate and purify on a small silica column eluting with 5% methanol/methylene chloride to give 15 mg (30%) of the free base of the title compound which has a retention time of 3.6 minutes and a mass of 509 (M+1) on LC/MS. Convert to the HCl salt and lyophilize.

### Preparation 15

### (2-Methoxy-3-{6-methoxy-1-[4-(2-piperidin-1-yl-ethoxy)-benzoyl]-naphthalen-2-yl}-phenyl)-acetic acid

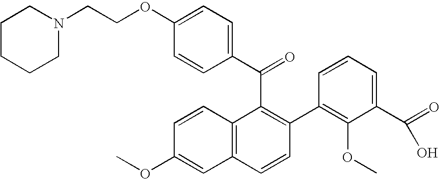

Place 6-methoxy-naphthalene-1-yl)-[4-(2-piperidin-1-yl-ethoxy)-phenyl]-methanone-2-boronic acid (403.0 mg, 0.930 mmol), 2-methoxy-3-bromobenzoic acid (444.8 mg, 1.93 mmol), sodium carbonate (791.3 mg, 7.47 mmol), and 10% Pd/C (˜100 mg) in absolute ethanol (20 mL). Place under nitrogen and reflux for 16 hours. Pass reaction through filtering agent and remove solvent. Take up residue in methanol and pass onto SCX resin. Wash resin with methanol and elute product with 2M ammonia in methanol. Remove the solvent and take up the material in 25% isopropanol/chloroform and wash with 1.0M HCl. Separate organic and extract aqueous with 25% isopropanol/chloroform (3×). Dry the combined organics with sodium sulfate and remove solvent. Separate by flash chromatography on silica gel (5-10% methanol/dichloromethane with 1% acetic acid). Scrape the resulting material in toluene and collect by filtration. Wash solid with ether and hexanes. Air dry to give 124.8 mg (24.9%) of the title compound.

### EXAMPLE 35

### 2-Hydroxy-5-[4-(2-piperidin-1-yl-ethoxy)-phenyl]-5H-6-oxa-chrysene-7-carboxylic acid trifluoroacetate

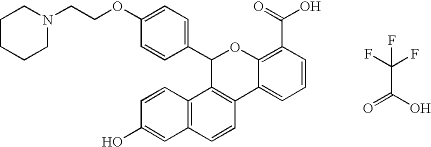

Dissolve (2-methoxy-3-{6-methoxy-1-[4-(2-piperidin-1-yl-ethoxy)-benzoyl]-naphthalen-2-yl}-phenyl)-acetic acid (124.8 mg, 0.231 mmol) and sodium t-butylthiolate (500.0 mg, 4.46 mmol) in dimethylformamide (20 mL). Place the resulting solution under nitrogen and heat to reflux for one half hour. Cool to room temperature, acidify to pH=2 with 1.0M HCl, and pass onto SCX resin. Wash resin with methanol and 2M ammonia in methanol. Collect all washes and remove solvent. Separate major product by HPLC and dissolve in dichloromethane (20 mL). Add trifluoroacetic acid (2.0 mL) and triethyl silane (2.0 mL). Stir at room temperature for one hour. Wash reaction with brine (50 mL) and separate the organic layer. Extract the aqueous portion with 25% isopropanol/chloroform (3×50 mL). Dry the combined organics with sodium sulfate and remove the solvent. Dissolve the residue in methanol and pass onto SCX resin. Wash the resin with methanol and elute the product with 2M ammonia in methanol. Isolate the title compound by HPLC and lyophilize to give 9.2 mg of the title compound: LCMS (8 min): 3.38 min, m/z=496 (M+1).

### Preparation 16

### 2,4-Bisbenzyloxy bromobenzene

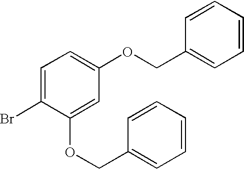

Charge a 1 liter flask with 500 ml dry dimethylformamide (DMF) and add 4-bromoresorcinol (9.5 grams, 0.05 moles) and start stirring. To this mixture add sodium hydride (60% in oil, 6 grams, 0.15 moles) in portions over ½ hour. To this mixture add benzyl bromide (29 grams, 0.168 moles) in portions over ½ hour. After two hours the reaction is complete as determined by TLC (silica gel, methylene chloride/hexane 1:1). Quench the reaction with ammonium chloride solution and remove the solvent on a rotavap at 80 degrees at which point the reaction mixture turns deep purple. Dissolve this in methylene chloride and wash three times with water, then 0.1 N sodium hydroxide then brine. Dry the solvent over 3A molecular sieves. Run the dark purple solution through a short plug of silica gel, which removes the purple color. Evaporate the filtrate to an oil and purify on a Biotage silica gel flash column, eluting excess benzyl bromide with 10% methylene chloride/hexane then elute the product with 20% methylene chloride/hexane. Evaporate the solvent to an oil, add methanol and chill overnight. In the morning filter the white crystals and air dry to give 11.2 g of 2,4-bisbenzyloxy bromobenzene (66%).

### EXAMPLE 36

### {2-Methoxy-5-[4-(2-piperidin-1-yl-ethoxy)-phenyl]-5H-6-oxa-chrysen-8-yl}-methanol

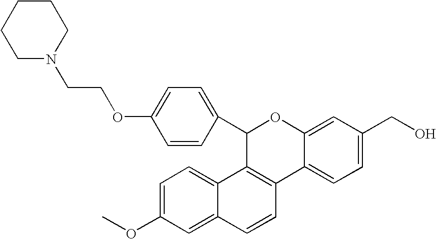

Dissolve 2-methoxy-5-[4-(2-piperidin-1-yl-ethoxy)-phenyl]-5H-6-oxa-chrysene-8-carboxylic acid methyl ester (46 mg. 0.09 mmoles) in 50 ml. THF and add 5 ml of 1.0 molar lithium aluminum hydride solution in THF. Stir for 30 minutes and check for completeness. Quench with sodium bicarbonate and extract the water layer two times with a 3/1 mixture of chloroform and isopropanol. Dry the solvent and evaporate to a glass. This material is used in the next step without purification.

### EXAMPLE 37

### 8-Hydroxymethyl-5-[4-(2-piperidin-1-yl-ethoxy)-phenyl]-5H-6-oxa-chrysen-2-ol

Dissolve {2-methoxy-5-[4-(2-piperidin-1-yl-ethoxy)-phenyl]-5H-6-oxa-chrysen-8-yl}-methanol in 25 ml. DMF and add excess sodium t-butyllthiolate. Heat the mixture under nitrogen at 110° C. for 18 hours. Neutralize the mixture with acetic acid and evaporate to a paste. Dissolve the material in methanol and purify on an SCX column, eluting with 2 N ammonia in methanol to give 26 mg of the title compound (62%). 1H-NMR (CD3OD, 400.00 MHz): 7.93 (d, J=8.8 Hz, 1H); 7.77 (dd, J=8.4, 3.2 Hz, 2H); 7.63 (d, J=9.2 Hz, 1H); 7.17 (d, J=2.8 Hz, 1H); 7.08 (s, 1H); 7.05 (d, J=3.6 Hz, 2H); 7.03 (d, J=2.8 Hz, 1H); 6.98 (s, 1H); 6.97-6.96 (m, 1H); 6.84 (d, J=1.2 Hz, 1H); 6.72 (s, 1H); 6.71 (d, J=12.0 Hz, 1H); 4.50 (s, 2H); 4.02-3.99 (t, 2H); 2.74-2.71 (t, 2H); 2.52 (m, 4H); 1.63-1.57 (m, 5H); 1.48-1.44 (m, 2H)

### EXAMPLES 38 and 39

### 8-Hydroxymethyl-5-[4-(2-piperidin-1-yl-ethoxy)-phenyl]-5H-6-oxa-chrysen-2-ol

The racemic mixture of 8-hydroxymethyl-5-[4-(2-piperidin-1-yl-ethoxy)-phenyl]-5H-6-oxa-chrysen-2-ol is separated on a Chiralpak AD column using 40% isopropanol/heptane mixture on a 0.46×25 cm column eluting at 1.0 ml/min. and monitoring at 225 nm. The compound that elutes first is Example 38 and the second that elutes is Example 39.

### EXAMPLE 40

### (4-{6-Methoxy-1-[4-(2-piperidin-1-yl-ethoxy)-phenoxy]-naphthalen-2-yl}-phenyl)-methanol

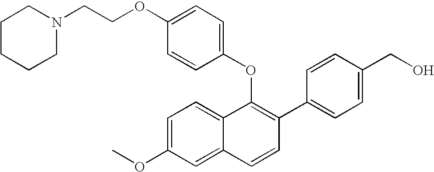

Combine trifluoromethanesulfonic acid 6-methoxy-1-[4-(2-piperidin-1-yl-ethoxy)-phenoxy]-naphthalen-2-yl ester (500 mg, 0.95 mmol), 4-hydroxymethylphenylboronic acid (435 mg, 2.85 mmol), K₂CO₃ (530 mg, 3.8 mmol), LiCl (160 mg, 3.8 mmol), toluene (10 ml), and water (1 ml), stir and bubble nitrogen into the slurry for 3 minutes. Add the catalyst, [1,1′-bis(diphenylphosphino)ferrocene] dichloropalladium(II), complex with dichloromethane (1:1) (390 mg, 0.48 mmol, 0.5 eq.), to the reaction mixture and heat to 90° C. After 18 hours, cool the reaction mixture to ambient temperature and dilute with diethyl ether (50 ml) and water (10 ml). Filter through a pad of celite and separate the layers. Wash the organic layer with brine (10 ml), dry with Na₂SO₄, filter, and concentrate in vacuo. Chromatograph the residue on a SiO₂ column eluting the material with methanol (0 to 7.5%) in dichloromethane to 10% methanol in dichloromethane (containing 0.5% NH₄OH) to give 321 mg (70%) of the title compound: Mass spectrum (ion spray): m/z=484.5 (M+H).

### 6-(4-Hydroxymethyl-phenyl)-5-[4-(2-piperidin-1-yl-ethoxy)-phenoxy]-naphthalen-2-ol, hydrochloride

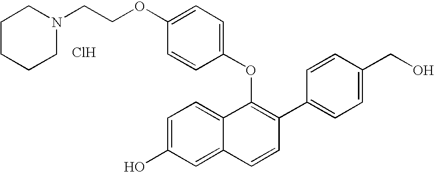

Combine (4-{6-methoxy-1-[4-(2-piperidin-1-yl-ethoxy)-phenoxy]-naphthalen-2-yl}-phenyl)-methanol (234 mg, 0.48 mmol), sodium ethanethiol (135 mg, 1.6 mmol), and N,N-dimethylformamide (10 mL). Heat solution to 160□C for 8 hours. Cool the reaction mixture to ambient temperature and dilute with water (70 mL) and ethyl acetate (50 mL). Separate the layers and extract the aqueous layer with ethyl acetate (50 mL). Combine the organic layers, dry with Na₂SO₄, filter, and concentrate in vacuo. Chromatograph the residue on a SiO₂ column eluting the material with methanol in dichloromethane (0 to 14%) to give 135 mg of the free base of the title compound. Dissolve the free base in ethyl acetate (2 mL) and methanol (0.2 mL) and dilute with diethyl ether (5 mL). Cool in an ice bath and treat with 2M HCl in diethyl ether (0.22 mL, 0.44 mmol). Dilute the reaction mixture with diethyl ether (25 mL) and collect the solid on filter paper. Rinse with diethyl ether and dry at 65° C. for 48 hours in vacuo (<2 mm of Hg) to give 124 mg (85%) of the title compound: Mass spectrum (ion spray): m/z=470.5 (M+H—HCl).

### EXAMPLE 42

### 1-(4-{6-Hydroxy-1-[4-(2-piperidin-1-yl-ethoxy)-phenoxy]-naphthalen-2-yl}-phenyl-ethanone

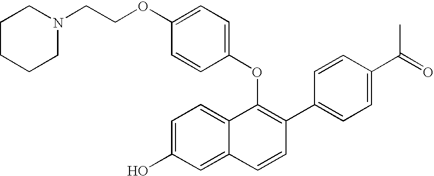

Combine trifluoro-methanesulfonic acid 6-methoxyoxy-1-[4-(2-piperidin-1-yl-ethoxy)-phenoxy]-naphthalen-2-yl ester (1 g, 1.9 mmol), 4-acetylphenylboronic acid (0.94 mg, 5.7 mmol), CsF (2.6 g, 17.1 mmol), and acetonitrile (20 ml). Add [1,1′-bis(diphenylphosphino)ferrocene]dichloropalladium(II), complex with dichloromethane (1:1) (775 mg, 0.95 mmol, 1 eq.) to the reaction mixture and heat to 90° C. After 22 hours, cool the reaction mixture to ambient temperature and evaporate the solvent. Dilute with diethyl ether (100 ml) and sonicate the mixture for 10 minutes. Filter through a pad of celite and concentrate the filtrate in vacuo. Chromatograph the residue on a SiO₂ column eluting the material with methanol in dichloromethane (0 to 6%) to give 844 mg (89%) of 1-(4-{6-methoxy-1-[4-(2-piperidin-1-yl-ethoxy)-phenoxy]-naphthalen-2-yl}-phenyl)-ethanone: Mass spectrum (ion spray): m/z=496.6 (M+H).

Combine 1-(4-{6-methoxy-1-[4-(2-piperidin-1-yl-ethoxy)-phenoxy]-naphthalen-2-yl}-yl}-phenyl)-ethanone (576 mg, 1.16 mmol) and pyridinium hydrochloride (7.5 g, 65 mmol) and heat to 200° C. Every 15 minutes add additional pyridinium hydrochloride (1 g) and monitor the reaction by mass spectroscopy. After 1.25 hours, cool the reaction mixture to ambient temperature and dissolve the residue in saturated aqueous NaHCO₃ (100 mL), ethyl acetate (250 mL) and methanol (10 mL). Separate the layers and extract the aqueous layer with a mixture of methanol (5 mL) and ethyl acetate (100 mL). Combine the organic layers, wash with water (50 mL), dry with Na₂SO₄, filter and concentrate. Triturate the crude reaction material with ethyl acetate (60 mL), filter away the solids and concentrate the filtrate. Chromatograph the residue on a SiO₂ column eluting with methanol in dichloromethane (0 to 10%) to give 317 mg (57%) of the title compound: Mass spectrum (ion spray): m/z=482.5 (M+H).

### EXAMPLE 43

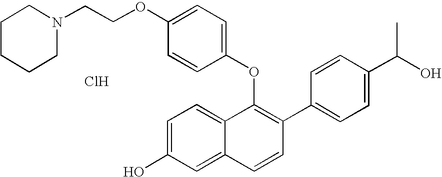

### 6-[4-(1-Hydroxy-ethyl)-phenyl]-5-[4-(2-piperidin-1-yl-ethoxy)-phenoxy]-naphthalen-2-ol hydrochloride

Dissolve 1-(4-{6-hydroxy-1-[4-(2-piperidin-1-yl-ethoxy)-phenoxy]-naphthalen-2-yl}-phenyl)-ethanone (162 mg, 0.34 mmol) in THF (20 mL) and cool in an ice bath. Add 1M LAH in THF (0.9 mL, 0.9 mmoL) and stir for 1 hour. Sequentially add water (150 mL), 15% aqueous NaOH (35 mL), and ethyl acetate (40 mL) to the reaction mixture.

Filter the slurry through packed celite and separate the biphasic filtrate. Wash the organic layer with water (2×5 mL) and brine (5 mL), dry with Na₂SO₄, filter, and concentrate in vacuo to obtain 146 mg of the free base of the title compound. Dissolve in ethyl acetate (2 mL) and dilute with diethyl ether (20 mL). Cool in an ice bath and treat with 2M HCl in diethyl ether (0.17 mL, 0.34 mmol). Dilute the reaction mixture with diethyl ether (25 mL) and collect the solid on filter paper. Rinse with diethyl ether and dry at 65° C. for 48 hours in vacuo (<2 mm of Hg) to give 94 mg (54%) of the title compound: Mass spectrum (ion spray): m/z=466.5 (M+H—HCl—H₂O).

### EXAMPLE 44

### 2-(4-{6-Methoxy-1-[4-(2-piperidin-1-yl-ethoxy)-phenoxy]-naphthalen-2-yl}-phenyl-propan-2-ol

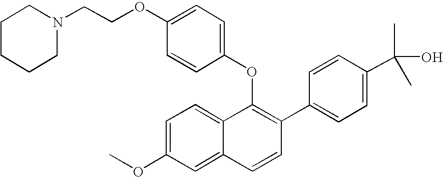

Dissolve 1-(4-{6-methoxy-1-[4-(2-piperidin-1-yl-ethoxy)-phenoxy]-naphthalen-2-yl}-phenyl)-ethanone (300 mg, 0.6 mmol) in diethyl ether (25 mL) and treat dropwise with a 1.4M solution of methyl magnesium bromide (1.9 mL, 4 mmol). Stir for 24 hours and then slowly quench with saturated aqueous ammonium chloride (25 mL). Dry the organic layer with Na₂SO₄, filter, and concentrate in vacuo. Chromatograph the residue on a SiO₂ column eluting the material with methanol in dichloromethane (0 to 6%) to give 306 mg (75%) of the title compound: ¹H NMR (CDCl₃): 7.86 (d, 1H), 7.68 (d, 1H), 7.55 (d, 1H), 7.51-7.54 (m, 2H), 7.42-7.45 (m, 2H), 7.19 (d, 1H), 7.08 (dd, 1H), 6.60-6.68 (m, 4H), 3.98-4.04 (m, 2H), 3.94 (s, 3H), 2.72-2.78 (m, 2H), 2.48-2.58 (m, 4H), 1.57-1.67 (m, 4H), 1.57 (s, 6H), 1.41-1.48 (m, 2H).

### EXAMPLE 45

### 6-[4-(1-Hydroxy-1-methyl-ethyl)-phenyl]-5-[4-(2-piperidin-1-yl-ethoxy)-phenoxy]-naphthalen-2-ol hydrochloride

Combine 2-(4-{6-methoxy-1-[4-(2-piperidin-1-yl-ethoxy)-phenoxy]-naphthalen-2-yl}-phenyl)-propan-2-ol (230 mg, 0.45 mmol), sodium ethanethiol (190 mg, 2.2 mmol), and N,N-dimethylformamide (10 mL). Heat solution to 160° C. for 1 hour. Cool the reaction mixture to ambient temperature and dilute with water (70 mL) and ethyl acetate (50 mL). Separate the layers and extract the aqueous layer with ethyl acetate (50 mL). Combine the organic layers, wash with brine (50 mL), dry with Na₂SO₄, filter, and concentrate in vacuo. Chromatograph the residue on a SiO₂ column eluting the material with methanol in dichloromethane (0 to 15%) to give 198 mg of the free base of the title compound. Dissolve the free base in ethyl acetate (2 mL) and dilute with diethyl ether (10 mL). Cool in an ice bath and treat with 2M HCl in diethyl ether (0.3 mL, 0.6 mmol) to obtain an off-white solid. Dilute the reaction mixture with diethyl ether (25 mL) and collect the solid on filter paper. Rinse with diethyl ether and dry at 65° C. for 48 hours in vacuo (<2 mm of Hg) to give 90 mg (37%) of the title compound: Mass spectrum (ion spray): m/z=480.3 (M+H—H₂O—HCl).

### Preparation 17

### 6-Methoxy-3-[4-(2-piperidin-1-yl-ethoxy)-phenoxy]-2-bromobenzo[b]thiophene

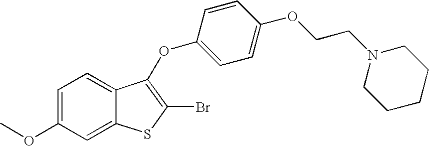

To a mixture of 3-methoxythiophenol and potassium carbonate in 850 mL of acetone is added dropwise bromoacetaldehyde diethyl acetal at room temperature. The heterogeneous mixture is stirred for 18 hrs and then filtered through a glass frit to remove salts. The filtered cake is washed (2×250 mL) with acetone and the filtrate is concentrated using a rotor evaporator. The filtrate is dissolved in diethyl ether (840 mL) and washed with water (850 mL), 1N NaOH (850 mL), and then brine (850 mL). The organic layer is dried over magnesium sulfate and concentrated using a rotor evaporator to give 212 g of a crude intermediate.

A 12 L flask is charged with 51 mL of boron trifluoride etherate and dissolved in 7.6 L of dichloromethane. 100 g of the crude intermediate prepared above is dissolved in 771 mL of dichloromethane and placed in a 1 L addition funnel. This mixture is added dropwise over the course of 30-45 min. After the addition is complete, the mixture is stirred for an additional hour and then 1 L of sat. sodium bicarbonate is added. The mixture is stirred until both layers are clear. The aqueous layer is extracted with an additional 500 mL of dichloromethane. The combined organic solutions are dried over magnesium sulfate and concentrated under a rotor evaporator (63.1 g crude). The residue is purified by the following protocol: 250 mL of heptane is added to the mixture and stirred for 15 min. This mixture is filtered through a silica gel plug which is washed with heptane (5×250 mL) and concentrated (40.83 g). The residue is distilled under vacuum (148° C./3 mm Hg) to provide 6-methoxybenzothiophene.

A 5 L flask is charged with 6-methoxybenzothiophene (25.26 g) and dissolved in 1.4 L of dichloromethane. m-CBPA (85 g) is added in portions over a 20-30 minute period. The mixture is heated to reflux for about 5 hours and the reaction monitored by HPLC. The mixture is cooled to room temperature and 950 mL of sodium hydrogen sulfite is added. The solution is stirred for 15 minutes. The aqueous layer is removed and the organic phase is washed with aqueous sodium bicarbonate (˜2×950 mL). The organic phase is separated, dried over magnesium sulfate and concentrated to give the sulfone compound as a greenish solid (26.56 g crude). Purification of the sulfone is conducted as follows: the crude material is first recrystallized from EtOH/hexanes to give 15.64 g of product (59% recovery). A second crop is recrystallized from EtOH to give 2.26 g of product, improving the recovery to 68%.

A flask is charged with 6-methoxybenzothiophene sulfone (6.31 g) and dissolved in 115 mL of chloroform. Bromine (dissolved in 10 mL of chloroform) is added dropwise over the course of 10 minutes. After about 4.5 hours TLC reveals consumption of starting material. The reaction is quenched by addition of triethylamine (5 mL). After stirring at room temperature for about 30 minutes, 450 mL of H₂O is added. The organic layer is separated and washed with 450 mL of brine, dried over magnesium sulfate and concentrated (11.50 g crude). After charcoal treatment, 7.73 g of 6-methoxy-2-bromobenzothiophene sulfone is isolated. The brominated sulfone is purified according to the following protocol: 50 mL of EtOH is added to the crude material and the mixture is heated to reflux for 45 minutes and brought to room temperature. After cooling in an ice bath for 30 minutes the solid is filtered through a glass frit and washed with cold EtOH (˜3×20 mL). 6-Methoxy-2-bromobenzothiophene sulfone (6.29 g) is recovered as a first crop (81%).

A flask is charged with 6-methoxy-2-bromobenzothiophene sulfone (8.05 g) and 100 mL of chloroform is added. Bromine (7.0 g, 1.5 eq.) in 50 mL of chloroform is added via addition funnel over the course of 20-30 minutes. After stirring for about 13 hours HPLC shows 3.5% starting material. 10 mL of triethylamine is added. After stirring at room temperature for 4 hours, 450 mL of H₂O is added and the organic layer is extracted. The organic layer is washed with 450 mL of brine and subsequently dried over magnesium sulfate and concentrated to give 6-methoxy-2,3-dibromobenzothiophene sulfone as a brownish solid. The dibrominated sulfone compound is purified according to the following protocol: 70 mL of EtOH is added to the compound and the mixture is heated to reflux for 45 minutes. The hot solution is cooled to room temperature and placed in an ice bath for 30 minutes. The crystals are filtered through a glass frit and washed with several portions of cold EtOH (˜3×20 mL) to give the dibrominated product (8.18 g) in 79% overall yield.

A flask is charged with 6-methoxy-2,3-dibromobenzothiophene sulfone (11.42 g) and 311 mL of THF is added. The temperature is reduced to 5° C. and the mixture is stirred at this temperature for about 15 minutes. Solid 4-(2-piperidin-1-yl-ethoxy)-phenol (7.84 g, 1.1 eq.) is added, followed by cesium carbonate (31.5 g, 3.0 eq.). The mixture is stirred for 15 minutes and then slowly brought to room temperature. After overnight stirring (13 hours), TLC reveals near consumption of starting material. 200 mL of H₂O is added followed by extraction with ethyl acetate (5×500 mL). The organic layers are combined and dried over magnesium sulfate. Solvent is removed under rotary evaporator to give 6-methoxy-3-[4-(2-piperidin-1-yl-ethoxy)-phenoxy]-2-bromobenzo[b]thiophene sulfone (14.47 g crude). The solid is purified by the following protocol: 100 mL of EtOH is added to a flask containing the solid and heated to reflux for 1 hour. The slurry is then allowed to cool to room temperature. The mixture is cooled in an ice bath for about 30-45 minutes. The solid is filtered and washed with cold EtOH. Based on the amount of initial crude material, the recovery as a first crop is about 83% (12.0 g).

6-Methoxy-2-bromo-3-[4-(2-piperidin-1-yl-ethoxy)-phenoxy]-2-bromobenzo[b]thiophene sulfone (150 g, 303 mmol) and 15 g of 10% Pd—C are combined with 1400 mL of THF. EtOH (1400 mL) is added and the mixture rapidly stirred while the vessel is evacuated arid purged with hydrogen several times. The reaction is stirred under hydrogen overnight at room temperature. Purge the reaction vessel with nitrogen, add Celite, stir, filter and rinse several times with MeOH. Remove the volatiles using a rotary evaporator, add Et₂O and concentrate to yield 6-methoxy-3-[4-(2-piperidin-1-yl-ethoxy)-phenoxy]benzo[b]thiophene sulfone. The product is purified by recrystallization from EtOH. This material is dissolved in methylene chloride and washed twice with saturated NaHCO₃, brine, then dried, filtered and concentrate to yield 112 g (89%) of 6-methoxy-3-[4-(2-piperidin-1-yl-ethoxy)-phenoxy]benzo[b]thiophene sulfone.

Dissolve 6-methoxy-3-[4-(2-piperidin-1-yl-ethoxy)-phenoxy]benzo[b]thiophene sulfone in 1.5 L of dioxane and add diisobutylaluminum hydride (1.617 L of a 1M solution in THF). Heat the solution to reflux for about 4 hours. Cool the solution to room temperature, slowly add 1 L of EtOAc, carefully transfer to a 12L sep funnel containing 4 L of 10% Rochelle salt (Na—K tartrate). Continued to add the rest of the reaction mixture slowly. Add 3 L of EtOAc, continue to stir until the mixture cools down. Add solid NaCl, stir and allow to settle overnight. Separate layers, and wash the organic layer with water (2×), then brine, dry over Na₂SO₄, filter and concentrate to yield 105 g. Purify by flash chromatography (2 kg of silica gel, 1%→5% MeOH/CH2Cl2) to yield 92.3 g (89%) of 6-methoxy-3-[4-(2-piperidin-1-yl-ethoxy)-phenoxy]benzo[b]thiophene.

Dissolve 6-methoxy-3-[4-(2-piperidin-1-yl-ethoxy)-phenoxy]benzo[b]thiophene in CH₂Cl₂ (950 mL). Add 13.37 mL of Br₂ in CH₂Cl₂ (50 mL) slowly. Allow the dark solution to stir for about 15 minutes at room temperature. Pour the mixture into 500 mL of a 10% aqueous Na₂S₂O₃ solution, separate and wash again with an additional 500 mL of Na₂S₂O₃ solution. Wash with saturated NaHCO₃ (1×500 mL, 1×300 mL), then brine. Dry over Na₂SO₄, filter and concentrate to yield 105 g of a dark oil. Purify by silica gel chromatography (3 kg of silica gel, 1→4% 2M NH3 in MeOH/CH₂Cl₂) to yield 96.25 g (88%) of the free base of title compound. Dissolve the residue in ˜500 mL of Et₂O and filter. Form the HCl salt by adding 104 mL of 2M HCl/Et₂O slowly to the rapidly stirring solution. Filter and wash with Et₂O 2× and dry to yield 99 g (96%) of the title compound.

### EXAMPLE 46

### 5-{6-Methoxy-3-[4-(2-piperidin-1-yl-ethoxy)-phenoxy]-benzo[b]thiophen-2-yl}-2,3-dihydro-isoindol-1-one

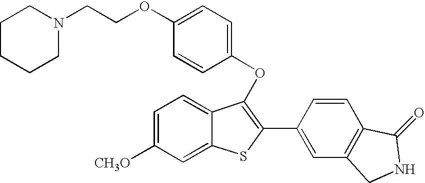

Combine 5-(4,4,5,5-tetramethyl-[1,3,2]dioxaborolan-2-yl)-2,3-dihydro-isoindol-1-one (740 mg, 2.9 mmol), 6-methoxy-3-[4-(2-piperidin-1-yl-ethoxy)-phenoxy]-2-bromobenzo[b]thiophene (473.9 mg, 1.0 mmol), 2M aqueous sodium carbonate solution (3.3 mL, 6.7 mmol) and dioxane (11 mL) in a 100 mL flask with septum. Bubble nitrogen gas through the mixture for 10 minutes. Add palladium tetrakistriphenylphosphine (116 mg, 0.1 mmol) and heat in a 90° C. oil bath for 18 hours. Cool the suspension to room temperature and quench with saturated aqueous ammonium chloride solution (20 mL). Separate the layers and extract the aqueous layer with ethyl acetate (4×50 mL). Wash the combined the organic layers with brine, dry (Na₂SO₄) and filter. Concentrate the filtrate and pre-adsorb the crude product onto silica gel. Chromatograph the residue on a SiO₂ column eluting the material with methanol in dichloromethane (0 to 20%) to give 311 mg of the title compound (64%).

### EXAMPLE 47

### 5-{6-Methoxy-3-[4-(2-piperidin-1-yl-ethoxy)-phenoxy]-benzo[b]thiophen-2-yl}-2,3-dihydro-isoindol-1-one Hydrochloride

Dissolve 5-{6-methoxy-3-[4-(2-piperidin-1-yl-ethoxy)-phenoxy]-benzo[b]thiophen-2-yl}-2,3-dihydro-isoindol-1-one (311 mg, 0.6 mmol) in dichloromethane (2 mL). Treat the resulting solution with 1M HCl in diethyl ether (10 mL, 10 mmol). Concentrate the resulting suspension in vacuo to obtain 309 mg of the title compound (93%).

### EXAMPLE 48

### 5-{6-Hydroxy-3-[4-(2-piperidin-1-yl-ethoxy)-phenoxy]-benzo[b]thiophen-2-yl}-2,3-dihydro-isoindol-1-one Hydrochloride

Dissolve 5-{6-methoxy-3-[4-(2-piperidin-1-yl-ethoxy)-phenoxy]-benzo[b]thiophen-2-yl}-2,3-dihydro-isoindol-1-one hydrochloride (309 mg, 0.6 mmol) in dichloromethane (15 mL) and cool to 0° C. in an ice-bath. Treat solution with 1M boron tribromide in dichloromethane (2.4 mL, 2.4 mmol), drop wise over 5 minutes and stir for 1.5 hours at 0° C. Add saturated aqueous sodium bicarbonate solution (10 mL) at 0° C. and warm to room temperature. Separate the resulting layers and extract the aqueous layer with ethyl acetate and tetrahydrofuran (1:1, 5×15 mL). Wash the combined organic layers with brine, dry (Na₂SO₄) and filter. Concentrate the filtrate and pre-adsorb the crude product onto silica gel. Chromatograph the residue on a SiO₂ column eluting the material with methanol in dichloromethane (0 to 40%) to give 111 mg of 5-{6-Hydroxy-3-[4-(2-piperidin-1-yl-ethoxy)-phenoxy]-benzo[b]thiophen-2-yl}-2,3-dihydro-isoindol-1-one. Dissolve the free-base in dichloromethane (10 mL) and treat with 1M HCl in diethyl ether (20 mL, 20 mmol). Concentrate in vacuo to obtain 117 mg of the title compound (39%): mass spectrum (ion spray): m/z=501.0 (M+H—HCl).

### EXAMPLE 49

### 5-{6-Methoxy-3-[4-(2-piperidin-1-yl-ethoxy)-phenoxy]-benzo[b]thiophen-2-yl}-3H-isobenzofuran-1-one

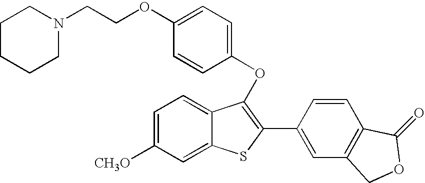

Combine 5-{6-methoxy-1-[4-(2-piperidin-1-yl-ethoxy)-phenoxy]-naphthalen-2-yl}-3H-isobenzofuran-1-one (242 mg, 0.9 mmol), 6-methoxy-3-[4-(2-piperidin-1-yl-ethoxy)-phenoxy]-2-bromobenzo[b]thiophene (234 mg, 0.5 mmol), 2M aqueous sodium carbonate solution (1.6 mL, 3.3 mmol) and dioxane (5 mL) in a 50 mL flask with septum. Bubble nitrogen gas through the reaction mixture for 10 minutes. Add palladium tetrakistriphenylphosphine (58 mg, 0.05 mmol) and heat in a 90° C. oil bath for 18 hours. Cool the suspension to room temperature and quench with saturated aqueous ammonium chloride solution (20 mL). Separate the layers and extract the aqueous layer with dichloromethane (3×20 mL). Wash the combined the organic layers with brine, dry (Na₂SO₄) and filter. Concentrate the filtrate and pre-adsorb the crude product onto silica gel. Chromatograph the residue on a SiO₂ column eluting the material with methanol in dichloromethane (0 to 15%) to give 110 mg of the title compound (46%).

### EXAMPLE 50

### 5-{6-Methoxy-3-[4-(2-piperidin-1-yl-ethoxy)-phenoxy]-benzo[b]thiophen-2-yl}-3H-isobenzofuran-1-one Hydrochloride

Dissolve 5-{6-methoxy-3-[4-(2-piperidin-1-yl-ethoxy)-phenoxy]-benzo[b]thiophen-2-yl}-3H-isobenzofuran-1-one (110 mg, 0.2 mmol) in dichloromethane (2 mL). Treat the resulting solution with 1M HCl in diethyl ether (10 mL, 10 mmol). Concentrate the resulting suspension in vacuo to obtain 117 mg of the title compound (99%).

### EXAMPLE 51

### 5-{6-Hydroxy-3-[4-(2-piperidin-1-yl-ethoxy)-phenoxy]-benzo[b]thiophen-2-yl}-3H-isobenzofuran-1-one Hydrochloride

Dissolve 5-{6-methoxy-3-[4-(2-piperidin-1-yl-ethoxy)-phenoxy]-benzo[b]thiophen-2-yl}-3H-isobenzofuran-1-one hydrochloride (117 mg, 0.2 mmol) in dichloromethane (6 mL) and cool to 0° C. in an ice-bath. Treat solution with 1M boron tribromide in dichloromethane (840 μL, 0.8 mmol), drop wise over 5 minutes and stir for minutes at 0° C. Add saturated aqueous sodium bicarbonate solution (5 mL) at 0° C. and warm to room temperature. Separate the resulting layers and extract the aqueous layer with ethyl acetate (5×15 mL). Wash the combined organic layers with brine, dry (Na₂₋SO₄) and filter. Concentrate the filtrate and pre-adsorb the crude product onto silica gel. Chromatograph the residue on a SiO₂ column eluting the material with methanol in dichloromethane (0 to 40%) to give 64 mg of 5-{6-Hydroxy-3-[4-(2-piperidin-1-yl-ethoxy)-phenoxy]-benzo[b]thiophen-2-yl}-3H-isobenzofuran-1-one. Dissolve the free-base in dichloromethane (10 mL) and treat with 1M HCl in diethyl ether (20 mL, 20 mmol). Concentrate in vacuo to obtain 68 mg of the title compound (60%): mass spectrum (ion spray): m/z=502.2 (M+H—HCl).

### Preperation 18

### 2-Benzyloxy-5-(4,4,5,5-tetramethyl-[1,3,2]dioxaborolan-2-yl)-benzoic acid methyl ester

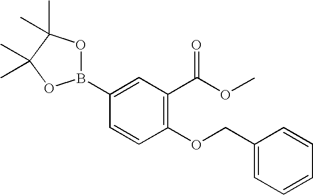

Combine 5-bromo-2-hydroxy-benzoic acid methyl ester 4.95 g, 21.4 mmol), benzyl alcohol (4.68 mL, 45.0 mmol) and triphenylphosphine (11.8 g, 45.0 mmol) in CH₂Cl₂ and add diisopropyl azodicarboxylate (8.86 mL, 45.0 mmol) dropwise over 15 minutes. Allow the mixture to stir at ambient temperature for 16 hours. Concentrate the reaction mixture in vacuo to an oil. Purify the residue by column chromatography using a silica gel column eluting with 4:1 hexane: ethyl acetate. Isolate 4.2 g (61%) 2-benzyloxy-5-bromo-benzoic acid methyl ester after concentrating the fractions.

Dissolve 2-benzyloxy-5-bromo-benzoic acid methyl ester (1.8 g, 5.6 mmol) and bis(pinicolato)diboron (1.57 g, 6.16 mmol) in dry dimethylsulfoxide (20 mL) and bubble nitrogen gas through the solution for 15 minutes. Then, add potassium acetate (1.7 g, 17.4 mmol) and dichloro[1,1′-bis(diphenylphosphino)ferrocene]palladium dichloromethane adduct (0.46 g, 0.56 mmol). Heat the mixture to 80° C. for 5 hours. Partition the reaction mixture between ethyl acetate (100 mL) and water (30 mL). Separate the organic layer and wash with brine solution (30 mL), dry over magnesium sulfate, filter and concentrate in vacuo. Purify the residue by column chromatography using a silica gel column eluting with 7:3 hexane:ethyl acetate to obtain 1.52 g (74%) of 2-benzyloxy-5-(4,4,5,5-tetramethyl-[1,3,2]dioxaborolan-2-yl)-benzoic acid methyl ester.

### EXAMPLE 52

### 2-Hydroxy-5-{6-hydroxy-1-[4-(2-piperidin-1-yl-ethoxy)-phenoxy]-naphthalen-2-yl}-N,N-dimethyl-benzamide Hydrochloride

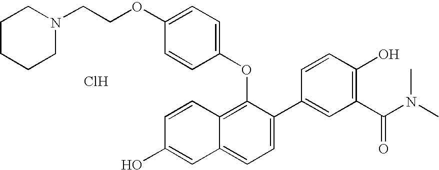

Combine trifluoro-methanesulfonic acid 6-methoxy-1-[4-(2-piperidin-1-yl-ethoxy)-phenoxy]-naphthalen-2-yl ester (0.56 g, 1.07 mmol) and 2-Benzyloxy-5-(4,4,5,5-tetramethyl-[1,3,2]dioxaborolan-2-yl)-benzoic acid methyl ester (0.826 g, 2.24 mmol) from preperation a in dry acetonitrile (25 mL). Bubble nitrogen through the solution for 15 minutes. Then add cesium fluoride (3.13 g, 20.6 mmol), palladium (II) acetate (0.18 g, 0.27 mmol) and tricyclohexylphosphine (0.11 g, 0.40 mmol) and fit with a reflux condenser. Heat the mixture in an oil bath preheated to 90° C. and stir for 45 minutes. Cool to ambient temperature and partition between CH₂Cl₂ (50 mL) and brine solution (30 mL). Separate the organic layer, dry over magnesium sulfate, filter and concentrate in vacuo. Purify the residue by column chromatography using a silica gel column eluting with 7:3 hexane:ethyl acetate+5% (7M NH₃/MeOH) to obtain 0.3 g (22%) 2-benzyloxy-5-{6-methoxy-1-[4-(2-piperidin-1-yl-ethoxy)-phenoxy]-naphthalen-2-yl}-benzoic acid methyl ester.

Dissolve 2-benzyloxy-5-{6-methoxy-1-[4-(2-piperidin-1-yl-ethoxy)-phenoxy]-naphthalen-2-yl}-benzoic acid methyl ester (0.30 g, 0.49 mmol) in methanol (10 mL) and treat the resulting solution with 5N aqueous sodium hydroxide solution (10 mL). Heat the mixture to 80° C. for 4 hours. Cool to ambient temperature and neutralize with 5N aqueous hydrochloric acid (10 mL). Partition the mixture between CH₂Cl₂ (100 mL) and brine solution (50 mL). Separate the organic layer, dry over magnesium sulfate, filter and concentrate in vacuo to obtain 0.29 g (100%) 2-benzyloxy-5-{6-methoxy-1-[4-(2-piperidin-1-yl-ethoxy)-phenoxy]-naphthalen-2-yl}-benzoic acid.

Combine 2-benzyloxy-5-{6-methoxy-1-[4-(2-piperidin-1-yl-ethoxy)-phenoxy]-naphthalen-2-yl}-benzoic acid (0.29 g, 0.45 mmol), 2M dimethyl amine/tetrahydrofuran (0.34 mL, 0.68 mmol), 1-hydroxybenzotriazole hydrate (0.09 g, 0.68 mmol), triethyl amine (0.22 ml, 1.6 mmol) and 1-(3-dimethylaminopropyl)-3-ethylcarbodiimide hydrochloride (0.43 g, 2.3 mmol) in CH₂Cl₂ (10 mL). Stir at ambient temperature for 256 hours. Dilute with CH₂Cl₂ (100 mL) and wash with saturated sodium bicarbonate solution (30 mL), water (30 mL), saturated ammonium chloride solution (30 mL), water (30 mL) and brine solution (30 mL). Separate the organic layer, dry over magnesium sulfate, filter and concentrate in vacuo. Purify the residue by column chromatography using a silica gel column eluting with 7:3 hexane:ethyl acetate+2% (7M ammonia/methanol) to obtain 0.21 g (76%) 2-Benzyloxy-5-{6-methoxy-1-[4-(2-piperidin-1-yl-ethoxy)-phenoxy]- naphthalen-2-yl}-N,N-dimethyl-benzamide. Dissolve 2-Benzyloxy-5-{6-methoxy-1-[4-(2-piperidin-1-yl-ethoxy)-phenoxy]-naphthalen-2-yl}-N,N-dimethyl-benzamide (0.21g, 0.33 mmol) in CH₂Cl₂ (5 mL). Add hydrogen chloride (0.5 mL, 1.0 M in ether) and stir the reaction mixture for 10 minutes. Concentrate in vacuo to obtain 2-Benzyloxy-5-{6-methoxy-1-[4-(2-piperidin-1-yl-ethoxy)-phenoxy]-naphthalen-2-yl}-N,N-dimethyl-benzamide hydrochloride (0.22 g, 100%)

Dissolve 2-Benzyloxy-5-{6-methoxy-1-[4-(2-piperidin-1-yl-ethoxy)-phenoxy]-naphthalen-2-yl}-N,N-dimethyl-benzamide hydrochloride (0.22 g, 0.33 mmol) in CH₂Cl₂ (15 mL). Cool the solution to 0° C. and add BBr₃ (2.16 mL, 2.16 mmol, 1M in CH₂Cl₂). Stir at 0° C. for 30 minutes, then warm to ambient temperature and stir an additional 2 hours. Partition the reaction mixture between CH₂Cl₂ (50 mL) and saturated sodium bicarbonate solution (35 mL). Separate the organic layer, dry over magnesium sulfate, filter and concentrate in vacuo. Purify the residue by column chromatography using a silica gel column eluting with 1:1 hexane:ethyl acetate+5% (7M ammonia/methanol) to obtain 0.09 g (49%) 2-Hydroxy-5-{6-hydroxy-1-[4-(2-piperidin-1-yl-ethoxy)-phenoxy]-naphthalen-2-yl}-N,N-dimethyl-benzamide: mass spectrum (ion spray): m/z=527.2 (M+H).

Add hydrogen chloride (0.25 mL, 1.0 M in ether) and stir the reaction mixture for 10 minutes. Concentrate in vacuo to obtain the title compound: mass spectrum (ion spray): m/z=527.2 (M+H—HCl).

### EXAMPLE 53

### (4-{6-Hydroxy-3-[4-(2-piperidin-1-yl-ethoxy)-phenoxy]-benzo [b]thiophen-2-yl}-phenyl)-phenyl-methanone hydrochloride

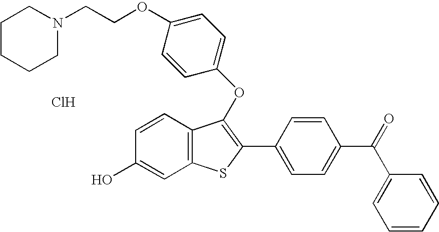

Combine 6-methoxy-3-[4-(2-piperidin-1-yl-ethoxy)-phenoxy]-2-bromobenzo[b]thiophene (0.41 g, 0.81 mmol), 4-benzoyl phenylboronic acid (0.37 g, 1.63 mmol) and tetrakis(triphenylphosphene)palladium(0) (0.09 g, 0.08 mmol) in 1,4-dioxane (20 mL) and bubble nitrogen through the solution for 15 minutes. Add 2M aqueous sodium carbonate solution (0.85 mL, 1.7 mmol) and heat the reaction mixture to 100° C. for 2 hours. Cool to ambient temperature and partition between saturated aqueous ammonium chloride solution (50 mL) and ethyl acetate (100 mL). Separate the organic layer and wash with saturated sodium bicarbonate solution (40 mL), water (40 mL) and brine solution (40 mL). Dry organic layer over magnesium sulfate, filter and concentrate in vacuo. Purify the residue by column chromatography using a silica gel column eluting with 1:1 hexane:ethyl acetate+2% 7M ammonia/methanol to obtain 0.28 g (61%) (4-{6-Methoxy-3-[4-(2-piperidin-1-yl-ethoxy)-phenoxy]-benzo[b]thiophen-2-yl}-phenyl)-phenyl-methanone.

Dissolve (4-{6-methoxy-3-[4-(2-piperidin-1-yl-ethoxy)-phenoxy]-benzo[b]thiophen-2-yl}-phenyl)-phenyl-methanone (0.28 g, 0.5 mmol) in CH₂Cl₂ (10 mL) and treat with hydrogen chloride (0.75 mL, 1.0 M in ether) and stir the reaction mixture for 10 minutes. Concentrate in vacuo to obtain 0.30 g (100%) (4-{6-Methoxy-3-[4-(2-piperidin-1-yl-ethoxy)-phenoxy]-benzo[b]thiophen-2-yl}-phenyl)-phenyl-methanone hydrochloride.

Dissolve (4-{6-methoxy-3-[4-(2-piperidin-1-yl-ethoxy)-phenoxy]-benzo[b]thiophen-2-yl}-phenyl)-phenyl-methanone hydrochloride (0.30 g, 0.5 mmol) in CH₂Cl₂ (20 mL). Cool the solution to 0° C. and add BBr₃ (1.89 mL, 1.89 mmol, 1M in CH₂Cl₂). Stir at 0° C. for 30 minutes, then warm to ambient temperature over 2 hours and stir another 2 hours. Partition between CH₂Cl₂ (100 mL) and saturated aqueous sodium bicarbonate solution (20 mL). Separate the organic layer and wash with brine solution (50 mL), dry over magnesium sulfate, filter and concentrate in vacuo. Purify the residue by column chromatography using a silica gel column eluting with 1:1 hexane:ethyl acetate+2% 7M ammonia/methanol to obtain 0.093 g (31%) (4-{6-Hydroxy-3-[4-(2-piperidin-1-yl-ethoxy)-phenoxy]-benzo[b]thiophen-2-yl}-phenyl)-phenyl-methanone: mass spectrum (ion spray): m/z=550.2 (M+H).

Dissolve (4-{6-hydroxy-3-[4-(2-piperidin-1-yl-ethoxy)-phenoxy]-benzo[b]thiophen-2-yl}-phenyl)-phenyl-methanone (0.093 g, 0.17 mmol) in CH₂Cl₂ (10 mL) and treat with hydrogen chloride (0.25 mL, 1.0 M in ether) and stir the reaction mixture for 10 minutes. Concentrate in vacuo to obtain 0.095 g (96%) of the title compound: mass spectrum (ion spray): m/z=550.2 (M+H—HCl).

### EXAMPLE 54

### (3-{6-Methoxy-1-[4-(2-piperidin-1-yl-ethoxy)-phenoxy]-naphthalen-2-yl}-phenyl)-morpholin-4-yl-methanone

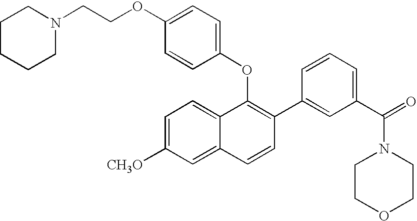

Combine trifluoro-methanesulfonic acid 6-methoxy-1-[4-(2-piperidin-1-yl-ethoxy)-phenoxy]-naphthalen-2-yl ester (300 mg, 0.57 mmol), 3-(morpholine-4-carbonyl)phenylboronic acid (282 mg, 1.20 mmol), dioxane (10 mL), tetrakis(triphenylphosphine)palladium (150 mg, 0.13 mmol) and 2M aqueous sodium carbonate (2 mL, 4 mmol) in a 250 mL flask fitted with a reflux condenser. Heat in a 90° C. oil bath for 4 hours, concentrate in vacuo, redissolve in ethyl acetate (150 mL), wash sequentially with aqueous saturated sodium bicarbonate (100 mL) and brine (100 mL), dry (Na₂SO₄), filter, and concentrate. Wash through SCX resin, eluting first with methanol, then 7N ammonia in methanol. Combine the fractions from the ammonia in methanol wash and concentrate in vacuo to afford 350 mg of a mixture of the title compound (>99%) and minor impurities that is used without further purification.

### EXAMPLE 55

### (3-{6-Methoxy-1-[4-(2-piperidin-1-yl-ethoxy)-phenoxy]-naphthalen-2-yl}-phenyl)-morpholin-4-yl-methanone hydrochloride

Dissolve (3-{6-methoxy-1-[4-(2-piperidin-1-yl-ethoxy)-phenoxy]-naphthalene-2-yl}-phenyl)-morpholin-4-yl-methanone (323 mg, 0.57 mmol) in dichloromethane (15 mL). Treat the resulting solution with 2M HCl in diethyl ether (2.8 mL, 5.6 mmol). After 10 minutes, concentrate in vacuo to obtain 344 mg of the title compound (>99%).

### EXAMPLE 56

### (3-{6-Hydroxy-1-[4-(2-piperidin-1-yl-ethoxy)-phenoxy]-naphthalen-2-yl}-phenyl)-morpholin-4-yl-methanone hydrochloride

Dissolve (3-{6-methoxy-1-[4-(2-piperidin-1-yl-ethoxy)-phenoxy]-naphthalen-2-yl}-phenyl)-morpholin-4-yl-methanone hydrochloride (334 mg, 0.57 mmol) in dichloromethane (15 mL) and cool to 0° C. in an ice bath. Add boron tribromide (210 μL, 2.2 mmol) and stir for 2 hours at 0° C. Add saturated aqueous sodium bicarbonate solution (20 mL) at 0° C. and warm to room temperature. Separate the resulting layers and extract the aqueous layer with dichloromethane (2×100 mL). Wash the combined organics with brine (125 mL), dry (Na₂SO₄), filter and concentrate. Flash chromatograph on silica gel eluting with 65:30:5 ethyl acetate/hexanes/(7N ammonia in methanol) to afford 245 mg of (3-{6-hydroxy-1-[4-(2-piperidin-1-yl-ethoxy)-phenoxy]-naphthalen-2-yl}-phenyl)-morpholin-4-yl-methanone. Dissolve the free-base (45 mg, 0.08 mmol) in dichloromethane (2 mL) and treat with 2M HCl in diethyl ether (0.4 mL, 0.8 mmol). After 5 minutes, concentrate in vacuo to afford 47 mg the title compound (>99%): mass spectrum (APCI, negative mode): m/z=551 (M−H—HCl).

### EXAMPLE 57

### N-Cyclopentyl-4-{6-methoxy-3-[4-(2-piperidin-1-yl-ethoxy)-phenoxy]-benzo[b]thiophen-2-yl}-benzamide

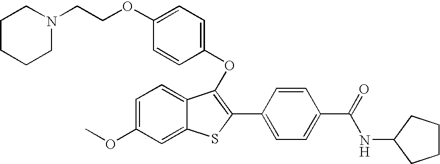

Mix 6-methoxy-3-[4-(2-piperidin-1-yl-ethoxy)-phenoxy]-2-bromobenzo[b]thiophene (300 mg, 0.5 mmol) and [4-(N-cyclopentylaminocarbonyl)phenyl]boronic acid (234 mg, 1 mmol) in acetonitrile (50 mL), bubble nitrogen through for 15 min. Add tricyclohexylphosphine (34 mg, 0.12 mmol), cesium fluoride (547 mg, 3.6 mmol), and palladium (II) acetate (15 mg, 0.067 mmol), sequentially. Heat the mixture at reflux for 4 hrs. Cool the reaction mixture to room temperature and load on a SCX column (60 mL), wash with methanol, then eluate the product with 2M ammonia methanol solution. Evaporate and separate on a silica gel column (40 g) eluting the material with 2M ammonia methanol in dichloromethane (0 to 5%) to give 205 mg of the title compound (43%). Mass spectrum (electron spray): m/z=571 (M+1).

### EXAMPLE 58

### N-Cyclopentyl-4-{6-hydroxy-3-[4-(2-piperidin-1-yl-ethoxy)-phenoxy]-benzo[b]thiophen-2-yl}-benzamide Hydrochloride

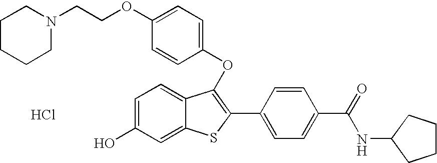

Dissolve N-cyclopentyl-4-{6-methoxy-3-[4-(2-piperidin-1-yl-ethoxy)-phenoxy]-benzo[b]thiophen-2-yl}-benzamide (130 mg, 0.23 mmol) in methylene chloride (5 mL), add 2M HCl in Et₂O (0.23 mL, 0.23 mol), evaporate and dry in vacuum. Dissolve the resultant foam in methylene chloride (10 mL) and cool to 0° C. under nitrogen. Add 1 M BBr₃ solution in methylene chloride (1.0 mL, 1.0 mmol) dropwise and stir for 10 minutes. Quench the reaction with methanol and partitioned between brine and ethyl acetate and dry the combined organic layers with sodium sulfate. Evaporate and separate on a silica gel column (40 g) eluting the material with 2M ammonia methanol in dichloromethane (0 to 5%) to give 79 mg of N-Cyclopentyl-4-{6-hydroxy-3-[4-(2-piperidin-1-yl-ethoxy)-phenoxy]-benzo[b]thiophen-2-yl}-benzamide (62%). Mass spectrum (electron spray): m/z=557 (M+1). Dissolve the free base in methylene chloride and add 1M HCl in Et₂O (0.14 mL, 0.14 mmol). Evaporate the solvent and dry in vacuum to give the title compound.

Prepare the compounds of Examples 59-65, illustrated in Table 1 below, as described above for the preparation of N-cyclopentyl-4-{6-methoxy-3-[4-(2-piperidin-1-yl-ethoxy)-phenoxy]-benzo[b]thiophen-2-yl}-benzamide.

TABLE 1

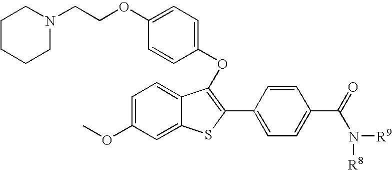

| | | | Mass Spectrum |
|---|---|---|---|
| Example | R⁸ | R⁹ | (Electron Spray) |
| 59 | H | Cyclopropyl | m/z = 543 (M + 1) |
| 60 | H | n-propyl | m/z = 545 (M + 1) |
| 61 | H | Methyl | m/z = 517 (M + 1) |
| 62 | H | Ethyl | m/z = 531 (M + 1) |
| 63 | Methyl | Methyl | m/z = 531 (M + 1) |
| 64 | H | H | m/z = 503 (M + 1) |

Prepare the compounds of Examples 65-70, illustrated in Table 1 below, as described above for the preparation of N-cyclopentyl-4-{6-hydroxy-3-[4-(2-piperidin-1-yl-ethoxy)-phenoxy]-benzo[b]thiophen-2-yl}-benzamide hydrochloride.

TABLE 1

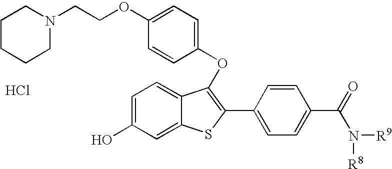

| | | | Mass Spectrum |
|---|---|---|---|
| Example | R⁸ | R⁹ | (Electron Spray) |
| 65 | H | Cyclopropyl | m/z = 529 (M + 1) |
| 66 | H | n-propyl | m/z = 531 (M + 1) |
| 67 | H | Methyl | m/z = 503 (M + 1) |
| 68 | H | Ethyl | m/z = 517 (M + 1) |
| 69 | Methyl | Methyl | m/z = 517 (M + 1) |
| 70 | H | H | m/z = 589 (M + 1) |

### Preparation 19

### 4-{6-tert-Butyl-diphenyl-silanyloxy)-3-[4-(2-piperidin-1-yl-ethoxy)-phenoxy]-benzo[b]thiophen-2-yl}-benzaldehyde

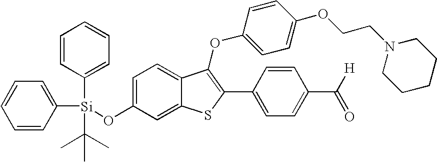

Add 6-methoxy-3-[4-(2-piperidin-1-yl-ethoxy)-phenoxy]-2-bromobenzo[b]thiophene (990 mg, 1.99 mmol) and dichloromethane (15 mL) to a round bottom flask. Cool the stirring solution to 0° C., and then add a 1.0 Molar solution of boron tribromide in dichloromethane (6.20 mL). Stir this dark brown solution for 2.5 hours at 0° C. to 10° C., and then cool the reaction to −78° C. Dilute the resulting mixture with diethyl ether (20 mL) and triethylamine (3.4 mL). Slowly add methanol (10 mL) then allow the stirring mixture to warm to ambient temperature. As mixture approaches ambient temperature, add ample saturated aqueous sodium bicarbonate. Extract the resulting mixture with ample ethyl acetate. Wash the combined extracts with saturated aqueous sodium bicarbonate, water and brine. Dry over sodium sulfate and concentrate in-vacuo. Obtained 911 milligrams of crude 2-bromo-3-[4-(2-piperidin-1-yl-ethoxy)-phenoxy]-benzo[b]thiophen-6-ol which is used as is in next reaction.

Combine 2-bromo-3-[4-(2-piperidin-1-yl-ethoxy)-phenoxy]-benzo[b]thiophen-6-ol (910 mg, 2.03 mmol), imidazole (311 mg, 4.57 mmol), N,N-dimethylaminopyridine (87 mg, 0.71 mmol) and dimethylformamide (21 mL) in a round bottom flask. Cool the mixture to 0° C. with stirring then slowly add tert-butylchlorodiphenylsilane (0.80 mL, 3.05 mmol) via syringe. Stir the reaction for approximately 15 hours at ambient temperature. Quench the reaction with brine and extract the resulting mixture with ample ethyl acetate. Wash the combined extracts with saturated aqueous sodium bicarbonate, water and brine. Dry over sodium sulfate and concentrate in-vacuo. Purify the residue by flash chromatography over silica gel (0-3% methanol gradient in chloroform) to obtain 1-(2-{4-[2-bromo-6-(tert-butyl-diphenyl-silanyloxy)-benzo[b]thiophen-3-yloxy]-phenoxy}-ethyl)-piperidine, 1.05 grams (77%).

Combine 1-(2-{4-[2-bromo-6-(tert-butyl-diphenyl-silanyloxy)-benzo[b]thiophen-3-yloxy]-phenoxy}-ethyl)-piperidine (1.42 g, 2.07 mmol), 4-formylphenylboronic acid (0.66 g, 4.41 mmol), tetrakis(triphenylphosphine)palladium(0) (0.24 g, 0.21 mmol), and 1,4-dioxane (25 mL) in a round bottom flask. Degas the resulting mixture under vacuum then purge with nitrogen. Add a 2M aqueous solution of sodium carbonate (2.30 mL, 4.62 mmol) and place the flask in a pre-heated oil bath at 105° C. Stir the reaction at reflux for 5-6 hours. Cool the reaction to ambient temperature and quench with saturated aqueous ammonium chloride. Extract the resulting mixture with ample ethyl acetate. Wash the combined extracts with saturated aqueous sodium bicarbonate, saturated aqueous ammonium chloride, water and brine. Dry over sodium sulfate and concentrate in-vacuo. Purify the resulting crude residue by flash chromatography over silica gel (0-5% methanol gradient in ethyl acetate) to obtain 1.20 g of the title compound (80%).

### EXAMPLE 71

### 4-{6-Hydroxy-3-[4-(2-piperidin-1-yl-ethoxy)-phenoxy]-benzo[b]thiophen-2-yl}-benzaldehyde

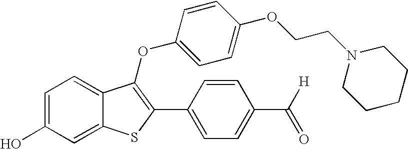

Place 4-{6-(tert-Butyl-diphenyl-silanyloxy)-3-[4-(2-piperidin-1-yl-ethoxy)-phenoxy]-benzo[b]thiophen-2-yl}-benzaldehyde (56 mg, 0.079 mmol) and tetrahydrofuran (3.0 mL) in a round bottom flask. Add a 1.0 Molar solution of tetrabutylammonium fluoride in tetrahydrofuran (0.094 mL, 0.094 mmol) to this stirring solution at ambient temperature. Stir the yellow colored reaction for 5-10 minutes at ambient temperature. Quench the reaction with saturated aqueous ammonium chloride; then dilute and extract the resulting mixture with ample ethyl acetate. Wash the combined extracts with saturated aqueous ammonium chloride, water and brine. Dry over sodium sulfate and concentrate in-vacuo. Purify the resulting crude solid by rotary chromatography over silica gel (4-9% methanol gradient in chloroform) to obtain 30 mg of the title compound (80%). MS (IS+) m/e 474 (M+1).

### EXAMPLE 72

### 2-(4-Hydroxymethyl-phenyl)-3-[4-(2-piperidin-1-yl-ethoxy)-phenoxy]-benzo[b]thiophen-6-ol, hydrochloride salt

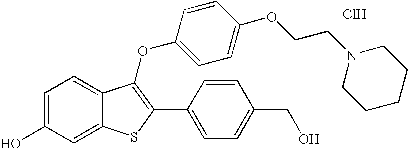

Combine 4-{6-hydroxy-3-[4-(2-piperidin-1-yl-ethoxy)-phenoxy]-benzo[b]thiophen-2-yl}-benzaldehyde (105 mg, 0.222 mmol), methanol (3.0 mL) and tetrahydrofuran (1.0 mL) in a round bottom flask. Add sodium borohydride (9 mg, 0.244 mmol) to this stirring solution at ambient temperature. Stir reaction for 30 minutes at ambient temperature then quench with water. Dilute the resulting mixture with ethyl acetate. Extract the resulting mixture with ample ethyl acetate. Wash the combined extracts with saturated aqueous sodium bicarbonate, water and brine. Dry over sodium sulfate and concentrate in-vacuo. Purify the resulting crude material by rotary chromatography over silica gel (5-10% methanol gradient in chloroform; then use 9% of a 2M ammonia/methanol solution in chloroform) to obtain 68 mg of the title compound (80%). MS (IS+) m/e 476 (M+1-HCl).

### EXAMPLE 73

### N-Isobutyl-4-{6-methoxy-3-[4-(2-piperidin-1-yl-ethoxy)-phenoxy]-benzo[b]thiophen-2-yl}-benzamide, hydrochloride salt

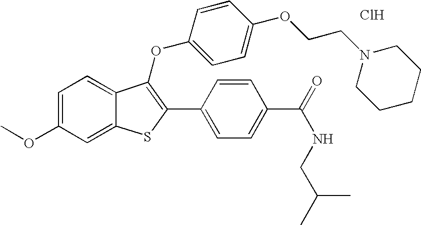

Combine 2-(4-hydroxymethyl-phenyl)-3-[4-(2-piperidin-1-yl-ethoxy)-phenoxy]-benzo[b]thiophen-6-ol, hydrochloride salt (275 mg, 0.551 mmol), 4-(isobutylaminocarbonyl)phenylboronic acid (244 mg, 1.10 mmol), tetrakis(triphenylphosphine)palladium(0) (64 mg, 0.055 mmol), and 1,4-dioxane (6.0 mL) in a round bottom flask. Degas the resulting mixture under vacuum then purge with nitrogen. Add a 2M aqueous solution of sodium carbonate (0.58 mL, 1.16 mmol) and place the flask in a pre-heated oil bath at 105° C. Stir the reaction at reflux for 3 hours. Add additional 4-(isobutylaminocarbonyl)phenylboronic acid (91 mg, 0.41 mmol), tetrakis(triphenylphosphine)palladium(0) (64 mg, 0.055 mmol), and 2M aqueous sodium carbonate (0.55 mL, 1.10 mmol), and stir the reaction overnight at reflux. Cool the reaction to ambient temperature and quench with saturated aqueous ammonium chloride. Extract the resulting mixture with ample ethyl acetate. Wash the combined extracts with saturated aqueous ammonium chloride, water and brine. Dry over sodium sulfate and concentrate in-vacuo. Purify the resulting crude solid on a 5 g SCX column, loading with dichloromethane and eluting with 2N ammonia/methanol. Dissolve the resulting material in dichloromethane (5 mL) and methanol (1 mL). Add 1.15 equivalents of a 2.0 Molar solution of hydrochloric acid in diethyl ether. Stir for 5 minutes at ambient temperature; then concentrate in vacuo to obtain 308 mg of the title compound (94%): MS (IS+) m/e 559 (M+1-HCl).

### EXAMPLE 74

### 4-{6-Hydroxy-3-[4-(2-piperidin-1-yl-ethoxy)-phenoxy]-benzo[b]thiophen-2-yl}-N-isobutyl-benzamide, hydrochloride salt

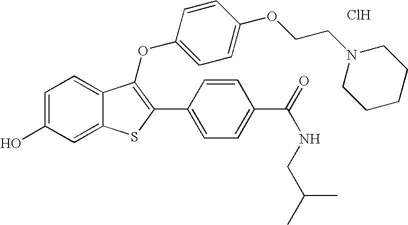

Add N-isobutyl-4-{6-methoxy-3-[4-(2-piperidin-1-yl-ethoxy)-phenoxy]-benzo[b]thiophen-2-yl}-benzamide, hydrochloride salt (300 mg, 0.504 mmol) and dichloromethane (7.5 mL) to a round bottom flask. Cool the stirring solution to 0° C.; then add a 1.0 Molar solution of boron tribromide in dichloromethane (1.61 mL, 1.61 mmol) via syringe. Stir the reaction for 3 hours allowing it to warm to room temperature slowly over that period. Quench the reaction with saturated aqueous sodium bicarbonate and add dichloromethane (5 mL). Stir this mixture for 20 minutes. Add saturated aqueous sodium potassium tartrate. Stir this mixture for 30 minutes. Extract the resulting mixture with ample dichloromethane. Wash the combined extracts with saturated aqueous sodium bicarbonate, water and brine. Dry over sodium sulfate and concentrate in-vacuo. Purify the resulting crude material by flash chromatography over silica gel (3.5-5% methanol gradient in chloroform). Dissolve the resulting material in dichloromethane (5 mL) and methanol (5 mL). Add 1.15 equivalents of a 2.0 Molar solution of hydrochloric acid in diethyl ether. Stir for 5 minutes at ambient temperature; then concentrate in vacuo to obtain 194 of the title compound (66%): MS (IS+) m/e 545 (M+1-HCl).

### Preparation 20

### 6-Boronic acid-5-[4-(2-piperidin-1-yl-ethoxy)-phenoxy]-naphthalen-2-ol hydrochloride salt

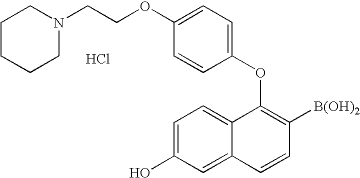

Charge a flask with trifluoro-methanesulfonic acid 6-methoxy-1-[4-(2-piperidin-1-yl-ethoxy)-phenoxy]-naphthalen-2-yl ester (10.0 g, 19.0 mmol) and dissolve in methylene chloride (100 mL). Add 2M HCl in ether (19 mL, 38 mmol) and remove solvent in vacuo. Redissolve in dry methylene chloride (200 mL) and cool to 0° C. under nitrogen. Add BBr₃ (9.0 mL, 95 mmol) slowly, and stir at 0° C. for 30 minutes. Pour reaction slowly into saturated aqueous sodium bicarbonate and extract with methylene chloride. Dry over sodium sulfate, filter and concentrate in vacuo. Dissolve crude material in methylene chloride (200 mL) and add N,N-diisopropylethylamine (16.5 mL, 95 mmol) and 4-dimethylaminopyridine (120 mg, 1.9 mmol) and stir at room temperature. Add acetic anhydride (3.6 mL, 38 mmol). Stir for 20 minutes and pour into saturated aqueous sodium bicarbonate. Extract with methylene chloride. Wash the organic layer with water, dry over sodium sulfate, filter and concentrate in vacuo to yield 10.5 g (100%) of acetic acid 5-[4-(2-piperidin-1-yl-ethoxy)-phenoxy]-6-trifluoromethanesulfonyloxy-naphthalen-2-yl ester.

Degas dry acetonitrile (100 mL) with nitrogen bubble for 10 minutes. Add palladium acetate (450 mg, 1.8 mmol), tricyclohexylphosphine (850 mg, 2.7 mmol) and cesium fluoride (11.6 g, 76 mmol) and stir for 20 minutes with degas. Add acetic acid 5-[4-(2-piperidin-1-yl-ethoxy)-phenoxy]-6-trifluoromethanesulfonyloxy-naphthalen-2-yl ester (5.6 g, 10.1 mmol) and stir under nitrogen for 3 minutes. Add bis(neopentyl glycolato)diboron (13.7 g, 60.6 mmol) and plunge into a 60° C. oil bath and stirred for 1 hr. Cool to room temperature and filter through celite and concentrate in vacuo. Dissolve the resulting solid in ether (100 mL) and add diethanolamine (1.0 g, 10.1 mmol) and stir for 1 hr. Filter the resulting white precipitate. Suspend the precipitate in water and add 1N HCl followed by methanol to dissolve the suspension. Stir for 36 hr. Extract with methylene chloride (x3), dry over sodium sulfate, filter and concentrate in vacuo to yield 2.7 g (66%) of the title compound. Mass spectrum (ion spray): m/z=408.2 (M+1-HCl).

### EXAMPLE 75

### 6-(3-Fluoro-4-hydroxymethyl-phenyl)-5-[4-(2-piperidin-1-yl-ethoxy)-phenoxy]-naphthalen-2-ol, trifluoroacetic acid salt

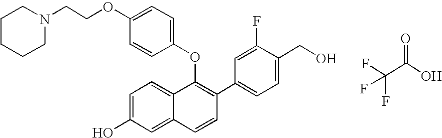

Add 6-boronic acid-5-[4-(2-piperidin-1-yl-ethoxy)-phenoxy]-naphthalen-2-ol hydrochloride salt (20 mg, 0.05 mmol) and freshly distilled dimethoxyethane and 2M sodium carbonate (9:1, 3 mL total volume) to a Quest210 under nitrogen. Add 3-fluoro-4-hydroxymethyl-bromobenzene (3 eq) followed by trans-dichlorobis(tri-o-tolylphosphine)palladium (10 mg, 0.01 mmol) and heat to 70° C. overnight under nitrogen. Cool reaction to room temperature and filter into tubes containing ˜400 mg TsOH-MP and agitate for 3 hours. Solvent filtered off and washed with DME. Add 3N ammonia in methanol and filter. Wash resin three times with 3N ammonia in methanol. Concentrate in vacuo and purify by reverse phase HPLC.

Preparative HPLC's may be obtained, e.g., on a Mass Guided Waters Preparative System using a 20×100 mm C18 Symmetry column. The eluent is a binary system of bottle and bottle A (0.1% trifluoroacetic acid in water) B (0.1% trifluoroacetic acid in acetonitrile). The standard method is a gradient of 10-95% B unless otherwise indicated. MS (IS+) m/e 488 (M+1-TFA)

Formulation

Because the free base form of a compound of formula I and the compound of formula II contain a basic moiety (i.e., amino), said compounds may be formulated as a pharmaceutical acid addition salt, e.g., as the hydrochloride salt or as a salt described in “Handbook of Pharmaceutical Salts: Properties, Selection and Use”, Weinheim, N.Y.: VHCA; Wiley-VCH, 2002.

The present pharmaceutical compositions are prepared by known procedures using well-known and readily available ingredients. In making the formulations of the present invention, the active ingredient (formula I compound) will usually be mixed with a carrier, or diluted by a carrier, or enclosed within a carrier which may be in the form of a capsule, sachet, paper or other container. When the carrier serves as a diluent, it may be a solid, semisolid or liquid material which acts as a vehicle, excipient or medium for the active ingredient.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents.

Biological Assays

Estrogen Receptor Binding Assay: Representative compounds of the present invention are screened for binding affinity to both estrogen receptor types (ERα and ERβ). This competition binding assay measures the compound's ability to displace ³H-estradiol and generates IC₅₀ and Kᵢ values for both receptor types.

This competition binding assay is run in a buffer containing 50 mM Hepes, pH 7.5, 1.5 mM EDTA, 150 mM NaCl, 10% glycerol, 1 mg/mL ovalbumin and 5 mM DTT, using 0.025 μCi per well ³H-Estradiol(NEN #NET517 at 118 Ci/mmol, 1 mCi/mL), 10 ng/well ERAlpha or ERbeta receptor (PanVera). A compound of the present invention is added at 10 different concentrations. Non-specific binding is determined in the presence of 1 μM of 17-B Estradiol. The binding reaction (140 μL) is incubated for 4 hours at room temperature, then 70 μl of cold DCC buffer is added to each reaction (DCC buffer contains per 50 mL of assay buffer, 750 mg of charcoal (Sigma) and 250 mg of dextran (Pharmacia)). Plates are mixed 8 minutes on an orbital shaker at 4° C. Plates are then centrifuged at 3,000 rpm at 4° C. for 10 minutes. An aliquot of 120 μl of the mix is transferred to another 96-well, white flat bottom plate (Costar) and 175 μl of Wallac Optiphase “Hisafe 3” scintillation fluid is added to each well. Plates are sealed and shaken vigorously on an orbital shaker. After an incubation of 2.5 hours, the plates are read in a Wallac Microbeta counter. The data is used to calculate an IC₅₀ and % Inhibition at 10 μM. The K_{d} for ³H-Estradiol is determined by saturation binding to ER alpha and ER beta receptors. The IC₅₀ values for test compounds are converted to Kᵢ using Cheng-Prusoff equation and the K_{d} determined by saturation binding assay.

Ishikawa Cell Proliferation Assay: This assay measures cell proliferation (using an alkaline phosphatase readout) in both an agonist mode in the presence of a compound of the present invention alone, and in an antagonist mode in which the ability of a compound of the present invention to block estradiol stimulation of growth is measured.

Ishikawa human endometrial tumor cells are maintained in MEM (minimum essential medium, with Earle's salts and L-Glutamine, Gibco BRL, Gaithersburg, Md.), supplemented with 10% fetal bovine serum (FBS) (V/V), (Gibco BRL). One day prior to assay, growth media is changed to assay medium, DMEM/F-12 (3:1) (Dulbecco's Modified Eagle Medium:Nutrient Mixture F-12, 3:1 Mixture, phenol red-free, Gibco BRL) supplemented with 5% dextran coated charcoal stripped fetal bovine serum (DCC-FBS) (Hyclone, Logen, Utah), L-Glutamine (2 mM), MEM sodium pyruvate (1 mM), HEPES (N-[2-hydroxyethyl]piperazine-N′-[2-ethanesulfonic acid] 2 mM) all from Gibco BRL). After an overnight incubation, Ishikawa cells are rinsed with Dulbecco's Phosphate Buffered Saline (1×) (D-PBS) without Ca⁺² and Mg⁺² (Gibco BRL), and trypsinized by a 3 minute incubation with 0.25% Trypsin/EDTA, phenol red-free (Gibco BRL). Cells are resuspended in assay medium and adjusted to 250,000 cells/mL. Approximately 25,000 cells in a 100 ul media are added to flat-bottom 96 wells microculture plates (Costar 3596) and incubated at 37° C. in a 5% CO₂ humidified incubator for 24 hours. The next day, serial dilutions of compounds are prepared in assay medium (at 6 times the final concentration in the assay). The assay is run in dual mode, agonist and antagonist modes.

For the agonist mode, plates receive 25 μl/well of assay medium followed by 25 μl/well of a diluted compound of the present invention (at 6× the final concentrations). For the antagonist mode, plates receive 25 μl/well of 6 nM E₂ (β-Estradiol, Sigma, St. Louis, Mo.) followed by 25 μl/well of a diluted compound of the present invention (at 6× the final concentrations). After an additional 48-hour incubation at 37° C. in a 5% CO₂ humidified incubator, media is aspirated from wells and 100 μl fresh assay medium is added to each microculture. Serial dilutions of compounds are prepared and added to the cells as described above. After an additional 72 hour incubation at 37° C. in a 5% CO₂ humidified incubator, the assay is quenched by removing media and rinsing plates twice in Dulbecco's Phosphate Buffered Saline (1×) (D-PBS) (Gibco BRL). The plates are dried for 5 minutes and frozen at −70° C. for at least 1 hour. The plates are then removed from the freezer and allowed to thaw at room temperature. To each well, 100 μl of 1-Step™ PNPP (Pierce Chemical Company, Rockford, Ill.) is added. After a 20-minute incubation, plates are read on a spectophotometer at 405 nm.

The data is fitted to a linear interpolation to derive EC₅₀ (for agonist mode) or IC₅₀ (for antagonist mode) values. For the antagonist mode, a % efficacy for each compound is calculated versus E2 (1 nM) alone. For the agonist mode, a % efficacy for each compound is calculated versus the response to tamoxifen.

In the agonist mode, the compounds of Examples 1, 2, 3, 5, 6, 8, 11, 35, 36, 37, 39, 41, 43 and 44 were tested and were found to be less stimulatory than tamoxifen. For example, the compound of Example 8 had a relative % efficacy of 15% and the compound of Example 35 had a relative % efficacy of 25%. In the antagonist mode, these same compounds inhibited greater than at least 80% of the 1 nM estradiol response. For example, the compound of Example 8 had an IC₅₀ of 9 nM and a % efficacy of 95% and the compound of Example 35 had an IC₅₀ of 36 nM and a % efficacy of 92%.

MCF-7 Proliferation Assay: The MCF-7 cell line is derived from a human breast adenocarcinoma and is used as an indicator of potential antiproliferative activity in breast epithelium.

MCF-7 breast adenocarcinoma cells (ATCC HTB 22) are maintained in MEM (minimal essential medium, phenol red-free, Gibco BRL) supplemented with 10% fetal bovine serum (FBS) (V/V), L-glutamine (2 mM), sodium pyruvate (1 mM), HEPES ((N-[2-hydroxyethyl]piperazine-N′-[2-ethanesulfonic acid] 10 mM}, non-essential amino acids (0.1 mM) and Penicillin Streptomycin (1×). Seven days prior to assay, MCF-7 cells are switched to assay media which is the same as maintenance medium except supplemented with 10% dextran-coated charcoal-stripped fetal bovine serum (DCC-FBS) assay medium in place of 10% FBS. MCF-7 cells are removed from flasks using 10× Trypsin EDTA (phenol red free, Gibco BRL) and diluted to 1× in (Ca++/Mg++ free HBSS (phenol red-free). Cells are adjusted to 80,000 cells/mL in assay medium. Approximately 8,000 cells (100 μl) are added to each well in 96 well Cytostar T scintillation plates (Amersham) and incubated at 37° C. in a 5% CO₂ humidified incubator for 24 hours to allow cell adherence and equilibration after transfer.

Serial dilutions of a compound of the present invention are prepared in assay medium at 4× the final desired concentration). A 50 μl aliquot of test compound dilutions (at 4× the final assay concentration) is transferred to duplicate wells followed by 50 μl assay medium for the agonist mode or 50 μl of 40 pM of E2 for the antagonist mode to a final volume of 200 μl. For each of the agonist plates, a basal level (media) and a maximum stimulated level (with 1 μM E2) is determined. For each of the antagonist plates, a basal level (media) and an E2 (10 pM) alone control is determined. After an additional 48 hours at 37° C. in a 5% CO₂ humidified incubator, 20 μl of assay medium containing 0.01 μCi of ¹⁴C-thymidine (52 mCi/mmol, 50 μCi/μl, Amersham) is added to each well. The plates are incubated overnight in the same incubator and then counted on the Wallac Microbeta counter. The data is averaged to calculate an IC₅₀ and % inhibition @ 1 μM for the antagonist mode. For the agonist mode, an EC₅₀ and percent of maximum E2 stimulation and concentration of maximum stimulation is calculated.

3-Day Rat Uterus Antagonist Assay: This model for uterine antagonism utilizes immature (3 week old) female rats that are highly sensitive to estrogenic stimulation of the uterus given that their circulating estrogen levels are prepubertal. The uteri from immature rats are fully responsive to exogenous estrogen, yet are quiescent in the absence of exogenous estrogen. Administration of exogenous estrogen to immature rats produces a reliable elevation of uterine weight, which can be used to study uterine antagonist effects. The rats are treated with both estradiol and 4 different concentrations of a compound of the present invention for 3 days and then uterine wet weights are measured.

Nineteen to twenty-one day old (or 45-50 g) female rats are orally treated with E2 (0.1 mg/kg, a maximal stimulatory estrogenic stimulus for reliably increasing uterine weight) and 10, 1.0, 0.1 and 0.01 mg/kg test compound for 3 days, 6 rats per group. Test compounds are dissolved in 20% β-hydroxycyclodextrin and administered by oral gavage in a volume of 0.2 mL daily (15 min. prior to the ethynyl estradiol gavage). A vehicle control, E2 alone and E2+raloxifene are also done as controls. The animals are fasted overnight following the final dose. On the following morning, the animals are weighed, then euthanized (by carbon dioxide asphyxiation) and the uteri rapidly collected (via a mid-line ventral incision) and weighed.

Uterine weight/body weight ratios (UWR) are calculated for each animal. The percent inhibition of the estrogen-induced response is then calculated by the following formula: percent inhibition=100×(UWR_{estrogen}−UWR_{test compound}/UWR_{estrogen}−UWR_{control}). ED₅₀ values are derived from a semi-log regression analysis of the linear aspect of the dose response curve. Both the UWR data and the percent inhibition data are statistically analyzed by one way analysis of variance (ANOVA) with post-hoc testing by Fisher's PLSD when indicated by a p≦0.05. Statistical analyses are performed using the Statview® 4.0 software package.

The compounds of Examples 5, 8, 11 and 37 were tested in the above assay and were found to inhibit the estrogen-induced response when administered at 1.0 mg/kg. For example, the compound of Example 11 had an ED₅₀ of 0.3 mpk and a % antagonism of 79% and the compound of Example 37 had an ED₅₀ of 0.06 mpk and a % antagonism of 89%

4-Day OVX Rat Uterine Agonist Assay: In order to assure that a test compound does not have any partial uterine agonist activity, compounds are administered to mature, ovariectomized rats.

Seventy-five day old rats are ovariectomized and treatment is started 14 days later when circulating estradiol levels have reached minimal levels. After 4 days of treatment with 3 doses of a compound of the present invention, (6 rats per group) body weight, uterine wet weight and uterine eosinophil peroxidase (EPO) activity are measured. Cholesterol levels are also measured to compare relative ability to lower cholesterol with other SERMs. If there is any question of uterine stimulation, histological examination will determine epithelial cell height.

The compound of Example 5 was tested in the above assay and did not cause any dose-related statistically significant increase in EPO activity.

10-Day Rat Hormone (Ovarian Stimulation) Screen: An initial, first screen for ovarian toxicity is conducted using a 10-day rat hormone study to measure estradiol and luteinizing hormone levels after compound administration. This screen is conducted by administering compound by oral gavage for 10 days to mature (9-10 week old) F344 female rats. Trunk blood is collected by rapid decapitation for evaluation of LH and estradiol levels approximately 2 hours after the 10^{th} dose. Serum, obtained by centrifugation, is removed and stored frozen below −60° C. until assayed. Serum levels of LH and estradiol are measured using radioimmunoassay (RIA) methods.

Rat LH primary antibody and reference preparations (rat LH:RP-3) are obtained from Dr. A. F. Parlow, Director, Pituitary Hormones and Antisera Center, Harbor-UCLA Medical Center, Torrance, Calif. The LH assay upper limits of detection are 30 ng/mL and the lower limits of detection are 0.1 ng/mL for the 100 μl samples.

E2 Clinical Assays. DiaSorin s.r.l., Saluggia (Vercelli), Italy. The upper limit of detection is 1000 pg/mL and the lower limit of detection is 5 pg/mL. The compounds of Examples 5 and 37 were tested in the above assay and did not significantly elevate circulating estradiol or LH levels.

35-Day Ovary-Intact Rat Bone Assay: While previous SERMs, including raloxifene have shown efficacy in preventing bone loss in OVX rats, the possibility of interference with estrogen-regulated turnover in ovary-intact rats needs to be addressed.

This assay is done in mature rats with concentrations based on the demonstrated efficacy in the 3-day assay. Generally, at least three concentrations are chosen based on multiples of the ED₅₀ generated therein. These multiples are generally 1×, 10× and 30× the ED₅₀. A compound of the present invention is administered to an OVX rat for 35 days and is compared to control, ovariectomized, and/or GnRH-administered rats. Femurs, tibiae, uteri, ovaries and serum are taken for further analyses. DEXA (Dual Energy X-ray Absorptivity), CT (Computed Tomography) and histologic analysis are done on the long bones to assess any changes. CT scans of the distal femur are done to calculate BMD (bone mineral density), cross sectional area and BMC (bone mineral content). Bone strength measurements (load to failure) may also be done to determine consequences of any bone mass or material changes. Uterine and ovarian histology are examined to confirm long term dosing effects of uterine efficacy and potential ovarian stimulation. The serum is analyzed for LH and E2 levels as a possible indicator of ovarian effects.

Utilities

The diseases, disorders or conditions for which a compound of formula I or II is useful in treating include, but are not limited to, (1) uterine cancer; (2) endometriosis; (3) uterine leiomyoma/leiomyomata; (4) post-menopausal osteoporosis, i.e., osteoporosis caused by the loss of bone that results from a lack of endogenous estrogen such as occurs in a woman following cessation of menstration due to natural, surgical, or other processes; and (5) estrogen receptor postive (ER+) breast cancer, particularly the prevention thereof. Treatment of uterine leiomyoma/leiomyomata as described herein, also contemplates the reduction of the occurrence or severity of the associated symptoms such as pain, urinary frequency, and uterine bleeding.

Dose

The specific dose administered is determined by the particular circumstances surrounding each situation. These circumstances include, the route of administration, the prior medical history of the recipient, the pathological condition or symptom being treated, the severity of the condition/symptom being treated, and the age of the recipient. The recipient patient's physician should determine the therapeutic dose administered in light of the relevant circumstances.

Generally, an effective minimum daily dose of a compound of formula I or II will exceed about 5 mg. Typically, an effective maximum daily dose will not exceed about 350 mg. The exact dose may be determined, in accordance with the standard practice in the medical arts of “dose titrating” the recipient; that is, initially administering a low dose of the compound, and gradually increasing the does until the desired therapeutic effect is observed.

1. A compound of formula I: 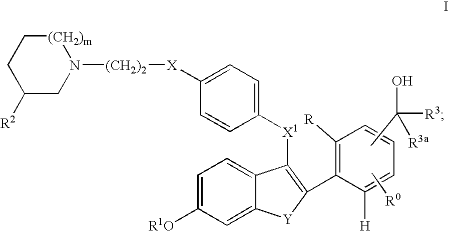
wherein:
m is 0, 1 or 2;
R⁰ is H, F or OH;
R¹ is H, SO₂(n-C₄-C₆ alkyl) or COR⁴;
R² is H or methyl provided that if m is 1 or 2, then R² must be H and that if m is 0, then R² must be methyl;
X is O or NR⁵;
Y is S or CH═CH;
R⁴ is C₁-C₆ alkyl, C₁-C₆ alkoxy, NR⁶R⁷, phenoxy, or phenyl optionally substituted with halo;
R⁵ is H or C₁-C₆ alkyl;
R⁶ and R⁷ are independently H, C₁-C₆ alkyl or phenyl;
R is H and X¹ is O, CH₂ or CO or R combines with X¹ to form a moiety of the formula: 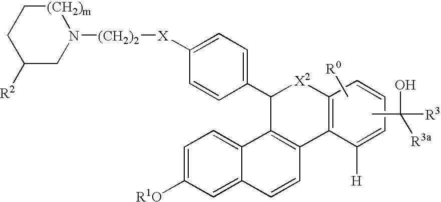
wherein X² is O or S; and
R³ and R^{3a} are independently H or C₁-C₆ alkyl; or a pharmaceutical acid addition salt thereof.

2. The compound of claim 1 wherein R⁰ is H.

3. The compound of claim 2 wherein R is H.

4. The compound of claim 3 wherein X and X¹ are O and m is 1 or 2.

5. The compound of claim 4 wherein R¹ is H or COR⁴ and R⁴ is C₁-C₄ alkyl, NHCH₃ or phenyl.

6. The compound of claim 5 wherein R¹ is H.

7. The compound of claim 6 wherein Y is CH═CH and m is 1.

8. The compound of claim 7 wherein R³ and R^{3a} are independently H or C₁-C₄ alkyl.

9. The compound of claim 8 wherein R³ and R^{3a} are independently H or methyl.

10. The compound of claim 9 wherein the COHR³R^{3a} moiety is at position 4.

11. The compound of claim 2 wherein R combines with X¹.

12. The compound of claim 11 wherein X and X² are O and m is 1 or 2.

13. The compound of claim 12 wherein R¹ is H or COR⁴ and R⁴ is C₁-C₄ alkyl, NHCH₃ or phenyl.

14. The compound of claim 13 wherein R¹ is H and m is 1.

15. The compound of claim 14 wherein R³ and R^{3a} are independently H or C₁-C₄ alkyl.

16. The compound of claim 15 wherein R³ and R^{3a} are independently H or methyl.

17. The compound of claim 16 wherein the COHR³R^{3a} moiety is at position 4.

18. (canceled)

19. A compound of formula II: 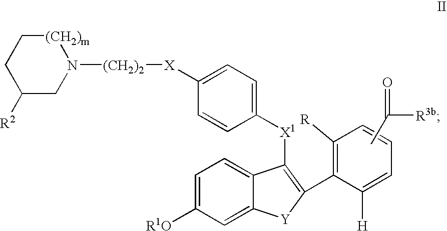
wherein:
m is 0, 1 or 2;
R¹ is H, SO₂(n-C₄-C₆ alkyl) or COR⁴;
R² is H or methyl provided that if m is 1 or 2, then R² must be H and that if m is 0, then R² must be methyl;
X is O or NR⁵;
Y is S or CH═CH;
R⁴ is C₁-C₆ alkyl, C₁-C₆ alkoxy, NR⁶R⁷, phenoxy, or phenyl optionally substituted with halo;
R⁵ is H or C₁-C₆ alkyl;
R⁶ and R⁷ are independently H, C₁-C₆ alkyl or phenyl;
R is H and X¹ is O or CH₂ or R combines with X¹ to form a moiety of the formula: 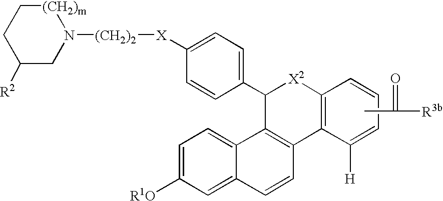
wherein X² is O or S;
R^{3b} is NR⁸R⁹ or OR¹⁰ or when R is H, R^{3b} may combine with the phenyl with which it is attached to form a moiety of the formula: 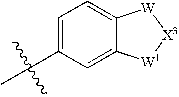
wherein W and W¹ are CH₂ or C═O provided that at least one of W or W¹ must be C═O; and X³ is NR¹¹ or O; and
R⁸ and R⁹ are independently H or C₁-C₆ alkyl or R⁸ and R⁹ may combine with the nitrogen to which they are both attached to form a morpholino, pyrollidino or piperidino ring;
R¹⁰ and R¹¹ are independently H or C₁-C₆ alkyl; or a pharmaceutical salt thereof.

20. The compound of claim 19 wherein R⁸ and R⁹ are independently H or C₁-C₆ alkyl.

21. The compound of claim 20 wherein X and X¹ are O and m is 1 or 2.

22. The compound of claim 20 or claim 21 wherein R¹ is H or COR⁴ and R⁴ is C₁-C₄ alkyl, NHCH₃ or phenyl.

23. The compound claim 22 wherein R¹ is H.

24. The compound of claim 23 wherein Y is CH═CH.

25. The compound of claim 24 wherein the COR^{3b} moiety is at the 3- or 4-position.

26. The compound of claim 25 wherein the COR^{3b} moiety is at the 4-position.

27. The compound of claim 26 wherein R^{3b} is NR⁸R⁹ and R⁸ and R⁹ are independently H or C₁-C₄ alkyl.

28. The compound of claim 26 wherein R^{3b} is OR¹⁰ and R¹⁰ is H or C₁-C₄ alkyl.

29. The compound of claim 26 wherein R is H and R^{3b} combines with the phenyl with which it is attached to form: 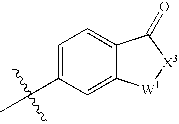
and W¹ is CH₂ and X³ is NR¹¹ and R¹¹ is H.

30. The compound of claim 26 wherein R is H and R³ combines with the phenyl with which it is attached to form: 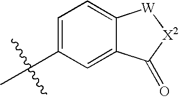
and R⁸ is H or C₁-C₄ alkyl.

31. The compound of claim 26 wherein R is H and R³ combines with the phenyl with which it is attached to form: 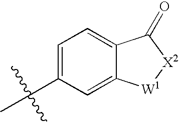
and R⁸ is H or C₁-C₄ alkyl.

32-35. (canceled)

36. A compound of formula III: 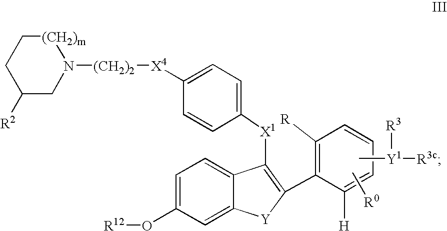
wherein:
m is 0, 1 or 2;
R⁰ is H, F or OH;
R² is H or methyl provided that if m is 1 or 2, then R² must be H and that if m is 0, then R² must be methyl;
Y is S or CH═CH;
Y¹ is C═O or C(OH);
R³ is H or C₁-C₆ alkyl;
R^{3c} is absent or is H or C₁-C₆ alkyl provided that if Y¹ is C(OH), then R^{3c} is H or C₁-C₆ alkyl and that if Y¹ is C═O, then R^{3c} is absent;
R¹² is H, C₁-C₆ alkyl, benzyl, SO₂CH₃, SO₂(n-C₄-C₆ alkyl) or COR⁴;
X⁴ is O or NR¹³;
R⁴ is C₁-C₆ alkyl, C₁-C₆ alkoxy, NR⁶R⁷, phenoxy, or phenyl optionally substituted with halo;
R⁶ and R⁷ are independently H, C₁-C₆ alkyl or phenyl;
R¹³ is H, C₁-C₆ alkyl or CO₂(C₁-C₆ alkyl); and
R is H and X¹ is O or CH₂ or R combines with X¹ to form a moiety of the formula: 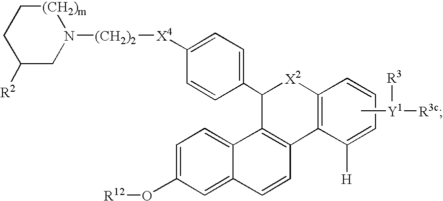
wherein X² is O or S;
provided that if Y¹ is C(OH), then R¹² is C₁-C₆ alkyl, SO₂CH₃ or benzyl or X⁴ is NR¹³ and R¹³ is CO₂(C₁-C₆ alkyl); or an acid addition salt thereof.

37. The compound of claim 36 wherein R⁰ is H.

38. The compound of claim 37 wherein R is H.

39. The compound of claim 38 wherein X⁴ and X¹ are O and m is 1 or 2.

40. The compound of claim 39 wherein R¹² is SO₂CH₃, benzyl or methyl.

41. The compound of claim 40 wherein Y is CH═CH and m is 1.

42. The compound of claim 41 wherein R³ and R^{3c} are independently H or C₁-C₄ alkyl.

43. The compound of claim 42 wherein R³ and R^{3c} are independently H or methyl.

44. The compound of claim 43 wherein the Y¹R³R^{3c} moiety is at position 4.

45. The compound of claim 37 wherein R combines with X¹.

46. The compound of claim 45 wherein X⁴ is O and m is 1 or 2.

47. The compound of claim 46 wherein R¹² is SO₂CH₃, benzyl or methyl.

48. The compound of claim 47 wherein X² is O and m is 1.

49. The compound of claim 48 wherein R³ and R^{3c} are independently H or C₁-C₄ alkyl.

50. The compound of claim 49 wherein R³ and R^{3c} are independently H or methyl.

51. The compound of claim 50 wherein the Y¹R³R^{3c} moiety is at position 4.

52. A compound of formula IV: 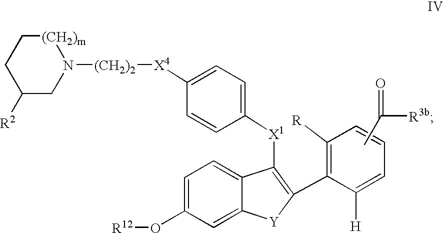
wherein:
m is 0, 1 or 2;
R² is H or methyl provided that if m is 1 or 2, then R² must be H and that if m is 0, then R² must be methyl;
Y is S or CH═CH;
R¹² is H, C₁-C₆ alkyl, benzyl, SO₂CH₃, SO₂(n-C₄-C₆ alkyl) or COR⁴;
R^{3b} is NR⁸R⁹ or OR¹⁰ or when R is H, R^{3b} may combine with the phenyl with which it is attached to form a moiety of the formula: 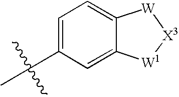
wherein W and W¹ are CH₂ or C═O provided that at least one of W or W¹ must be C═O; and X³ is NR¹¹ or O;
X⁴ is O or NR¹³;
R⁴ is C₁-C₆ alkyl, C₁-C₆ alkoxy, NR⁶R⁷, phenoxy, or phenyl optionally substituted with halo;
R⁶ and R⁷ are independently H, C₁-C₆ alkyl or phenyl;
R⁸ and R⁹ are independently H or C₁-C₆ alkyl or R⁸ and R⁹ may combine with the nitrogen to which they are both attached to form a morpholino, pyrollidino or piperidino ring;
R¹⁰ and R¹¹ are independently H or C₁-C₆ alkyl;
R¹³ is H, C₁-C₆ alkyl or CO₂(C₁-C₆ alkyl); and
R is H and X¹ is O, CH₂ or CO or R combines with X¹ to form a moiety of the formula: 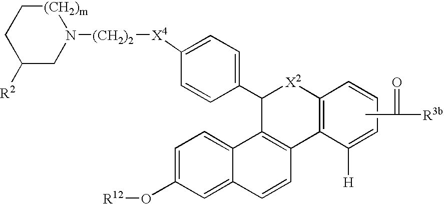
wherein X² is O or S;
provided that if R¹² is H, SO₂(n-C₄-C₆ alkyl) or COR⁴, then X⁴ is NR¹³ and R¹³ is CO₂(C₁-C₆ alkyl); or an acid addition salt thereof.

53. The compound of claim 52 wherein R⁸ and R⁹ are independently H or C₁-C₆ alkyl.

54. The compound of claim 53 wherein X⁴ and X¹ are O and m is 1 or 2.

55. The compound of claim 54 wherein R¹² is SO₂CH₃, benzyl or methyl.

56. The compound of claim 55 wherein Y is CH═CH.

57. The compound of claim 56 wherein the COR^{3b} moiety is at the 3- or 4-position.

58. The compound of claim 57 wherein the COR^{3b} moiety is at the 4-position.

59. The compound of claim 58 wherein R^{3b} is NR⁸R⁹ and R⁸ and R⁹ are independently H or C₁-C₄ alkyl.

60. The compound of claim 59 wherein R^{3b} is OR¹⁰ and R¹⁰ is H or C₁-C₄ alkyl.

61. The compound of claim 60 wherein R is H and R^{3b} combines with the phenyl with which it is attached to form: 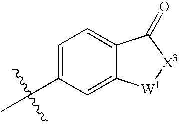
and W¹ is CH₂ and X³ is NR¹¹ and R¹¹ is H.

62. The compound of claim 60 wherein R is H and R^{3b} combines with the phenyl with which it is attached to form: 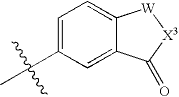
and R¹¹ is H or C₁-C₄ alkyl.

63. The compound of claim 60 wherein R is H and R^{3b} combines with the phenyl with which it is attached to form: 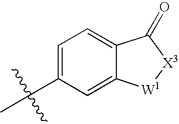
and R¹¹ is H or C₁-C₄ alkyl.
